(19) United States
(12) Patent Application Publication
(10) Pub. No.: US 2024/0108732 A1
(43) Pub. Date: Apr. 4, 2024

(54) Formulated and/or Co-Formulated Lipid Nanocarriers Compositions Containing Toll-Like Receptor ("TLR") Agonist Prodrugs Useful In The Treatment of Cancer and Methods Thereof

(71) Applicant: Nammi Therapeutics, Inc., Los Angeles, CA (US)

(72) Inventors: David Stover, Los Angeles, CA (US); Dhruba Bharali, Sherman Oaks, CA (US); Bruce A HAY, Niskayuna, NY (US); Tahmineh Safale, Yucca Valley, CA (US)

(21) Appl. No.: 18/445,036

(22) Filed: Mar. 10, 2023

Related U.S. Application Data
(60) Provisional application No. 63/372,416, filed on Mar. 10, 2022.

(51) Int. Cl. A61K 47/54; A61K 9/51; A61K 31/704; A61K 45/06

(52) U.S. Cl. CPC A61K 47/543; A61K 9/5123; A61K 31/704; A61K 45/06; A61K 47/542

(57) ABSTRACT

Formulated and/or co-formulated nanocarriers comprising TLR prodrugs and/or TLR Lipid Moieties and methods of making the nanocarriers are disclosed herein. The TLR prodrug compositions comprise a drug moiety, a lipid moiety, and linkage unit that inhibit Toll-Like Receptor (e.g., TLR1/2, TLR4, and/or TLR7). The TLR prodrugs can be formulated and/or co-formulated into a nanocarrier (e.g., LNP or SLNP) to provide a method of treating cancer, immunological disorders, and other disease by utilizing a targeted drug delivery vehicle.

### CROSS-REFERENCE TO RELATED APPLICATIONS

This application claims priority to U.S. Provisional Patent Application No. 63/372,416 filed 10 Mar. 2022, the contents of which are fully incorporated by reference herein.

### SUBMISSION OF SEQUENCE LISTING ON PAPER COPY AND XML FILE (“SEQUENCE LISTING XML”)

The content(s) of the following submissions are fully incorporated by reference herein in their entirety: a paper copy of the Sequence Listing recorded Mar. 9, 2023. Additionally, the content of a computer readable form (CRF) of the Sequence Listing in XML format submitted via the USPTO's patent electronic filing system (See, Section I.1 of the Legal Framework for Patent Electronic System in XML format) into the above-captioned application entitled (file name: 1551-20007.00—SEQ LIST—As-Filed 10 Mar. 2023—Updated 3 Nov. 23.XML, date recorded Nov. 3, 2023, size: 2.88 KB).

### STATEMENT OF RIGHTS TO INVENTIONS MADE UNDER FEDERALLY SPONSORED RESEARCH

Not applicable.

### FIELD OF THE INVENTION

The invention described herein relates to prodrug compositions that inhibit toll-like receptor(s) (“TLR”) after release of the active inhibitor from the prodrug and nano-formulations comprising such prodrugs. Specifically, the invention relates to prodrug compositions which are formulated within a nanocarrier (e.g., a liposome) and used as a vehicle for cancer therapy in humans. The invention also relates to co-formulations of such prodrugs with other immune-modulating agents or prodrugs. The invention further relates to the treatment of cancers and other immunological disorders and diseases.

### BACKGROUND OF THE INVENTION

Cancer is the second leading cause of death next to coronary disease worldwide. Millions of people die from cancer every year and in the United States alone cancer kills well over a half-million people annually, with 1,688,780 new cancer cases diagnosed in 2017 (American Cancer Society). While deaths from heart disease have been declining significantly, those resulting from cancer generally are on the rise. In the early part of the next century, cancer is predicted to become the leading cause of death unless medical developments change the current trend.

Several cancers stand out as having high rates of mortality. In particular, carcinomas of the lung (18.4% of all cancer deaths), breast (6.6% of all cancer deaths), colorectal (9.2% of all cancer deaths), liver (8.2% of all cancer deaths), and stomach (8.2% of all cancer deaths) represent major causes of cancer death for both sexes in all ages worldwide (GLOBOCAN 2018). These and virtually all other carcinomas share a common lethal feature in that they metastasis to sites distant from the primary tumor and with very few exceptions, metastatic disease fatal. Moreover, even for those cancer patients who initially survive their primary cancers, common experience has shown that their lives are dramatically altered. Many cancer patients experience strong anxieties driven by the awareness of the potential for recurrence or treatment failure. Many cancer patients also experience physical debilitations following treatment. Furthermore, many cancer patients experience a recurrence of their disease.

Although cancer therapy has improved over the past decades and survival rates have increased, the heterogeneity of cancer still demands new therapeutic strategies utilizing a plurality of treatment modalities. This is especially true in treating solid tumors at anatomical crucial sites (e.g., glioblastoma, squamous carcinoma of the head and neck and lung adenocarcinoma) which are sometimes limited to standard radiotherapy and/or chemotherapy. Nonetheless, detrimental effects of these therapies are chemo- and radio resistance, which promote loco-regional recurrences, distant metastases and second primary tumors, in addition to severe side-effects that reduce the patients' quality of life.

Toll-Like Receptors (“TLRs”) are a family of ten (10) identified pattern recognition receptors that respond to pathogen associated molecular patterns (PAMPs) and self-derived damage-associated molecule patterns (DAMPs). The TLRs then activate downstream pathways that initiate an innate immune response by producing inflammatory cytokines, type I interferon (IFN), and other mediators.

The TLR class of proteins are single, membrane-spanning, receptors usually expressed on sentinel cells such as macrophages and dendritic cells that recognize conserved molecules derived from microbes. Once these microbes have breach physical barriers (e.g., skin, or intestinal tract mucosa) they are recognized by TLRs, which then activate immune cell responses.

Upon activation, TLRs recruit adapter proteins (i.e., proteins that mediate other protein-protein interactions) within the cytosol of the immune cell in order to propagate the antigen-induced signal transduction pathway. These recruited proteins are then responsible for the subsequent activation of other downstream proteins, including protein kinases (IKKi, IRAK1, IRAK4, and TBK1) that further amplify the signal and ultimately lead to the upregulation or suppression of genes that orchestrate inflammatory responses and other transcriptional events.

Within the TLR family, several members are worth noting. TLR1 and TLR2 (“TLR 1/2”) are cell surface receptors that form heterodimers which recognize bacterial antigens such as lipoproteins as well as DAMPs such as HMGB1, heat shock proteins, and proteoglycans. TLR1/2 are expressed in pre-dendritic cells, macrophage, and NK cells where they mediate the innate response to PAMPS and DAMPS, upregulating inflammatory cytokines and enhancing antigen processing. TLR1/2 agonists, such as PAM3CSK4, can also enhance adaptive immunity as they have been shown to abrogate the immune suppressive effects of Treg cells.

Additionally, TLR4 is a cell surface receptor for various bacterial and viral components, most notably Lipopolysaccharide (LPS). LPS, also known as endotoxin, is a cell wall component of Gram-negative bacteria. LPS has been shown as a natural adjuvant for specific immune responses, especially antigen (Ag)-specific antibody and T cell responses. The toxicity associated with LPS has precluded its use as an effective and safe vaccine adjuvant. However, the monophosphorylated lipid A (MPLA), a metabolic product of LPS, has been found to maintain many of the immunostimulatory functions of LPS, but is significantly less toxic than its parent. Accordingly, MPLA works well as a safe and effective vaccine adjuvant. Lipid A structural features known to account for the maintenance of adjuvant properties and the loss of toxicity include the number of phosphate groups, as well as the number, type, and location of fatty acid residues. Synthetic MPLA, as well as a number of functional analogs, have been produced and characterized as immune stimulating adjuvants. A number of these have been used clinically as adjuvants in vaccine cocktails including in conjunction with tumor antigens to illicit anti-tumor immunity.

In addition, TLR4 is also a receptor for High Mobility Group Box 1 (HMGB1), a protein secreted by tumor cells upon immunogenic cell-death that enhances anti-tumor immunity by recruitment of dendritic cells and stimulation of antigen processing and secretion of inflammatory cytokines by antigen presenting cells (APCs). Accordingly, co-delivery of a TLR4 agonist with an ICD-inducing chemotherapy to a tumor enhances the anti-tumor immunity initiated by the ICD-chemotherapeutic.

Additionally, a prodrug is a medication or compound that, after administration, is metabolized converted within the body) into a pharmacologically active drug. Instead of administering a drug directly, a corresponding prodrug is used instead to improve how a medicine is absorbed, distributed, metabolized, and/or excreted. Prodrugs are often designed to improve bioavailability when a drug itself is poorly absorbed from the gastrointestinal tract, for example. A prodrug may be used to improve how selectively the drug interacts with cells or processes that are not its intended target. This reduces adverse or unintended effects of a drug, especially important in treatments like chemotherapy, which can have severe unintended and undesirable side effects. Prodrugs can thus be viewed as drugs containing specialized non-toxic protective groups used in a transient manner to alter or to eliminate undesirable properties in the parent molecule.

Finally, a nanocarrier is a nanomaterial being used as a transport for another substance, such as a drug. There are many different types of nanocarriers. For example, nanocarriers include polymer conjugates, polymeric nanoparticles, lipid-based carriers, and dendrimers to name a few. Different types of nanomaterial(s) being used in nanocarriers allows for hydrophobic and hydrophilic drugs to be delivered throughout the body. Since the human body contains mostly water, the ability to deliver hydrophobic drugs effectively in humans is a major therapeutic benefit of nanocarriers. Nanocarriers show promise in the drug delivery process because they can deliver drugs to site-specific targets, allowing drugs to be delivered in certain organs or cells but not in others. Site-specificity is a major therapeutic benefit since it prevents drugs from being delivered to the wrong places. Additionally, nanocarriers show specific promise for use in chemotherapy because they can help decrease the adverse, broader-scale toxicity of chemotherapy on healthy, fast-growing cells around the body. Since chemotherapy drugs can be extremely toxic to human cells, it is important that they are delivered to the tumor without being released into other parts of the body.

From the aforementioned, it will be readily apparent to those skilled in the art that a new treatment paradigm is needed in the treatment of cancers and other immunological diseases. By using novel prodrugs in conjunction with modern nanocarrier modalities, a new disease treatment can be achieved with the overall goal of more effective treatment(s), reduced side effects, and greater therapeutic utility in the treatment of cancers, especially the treatment of cancers in solid tumors.

Given the current deficiencies associated with cancer treatment, it is an object of the present invention to provide new and improved methods of treating cancer(s), immunological disorders, and other diseases utilizing prodrugs encapsulated within a nanocarrier.

In the present disclosure, use of TLR agonists in conjunction with ICD-inducing chemotherapeutics, PD-1 antagonists, additional toll receptor agonists, STING agonists, IDO inhibitors, CTLA4 inhibitors, CD1D agonists, TGFb inhibitors, A2aR inhibitors, and/or prodrugs thereof, to illicit an immune response directly against the actual patient's tumor cells in situ (i.e., without the need to introduce a tumor antigen or to remove tumor cells for ex vivo treatment). These synergistic functional agents are packaged into a single nano-carrier vehicle ensuring co-delivery and enhanced tumor selectivity of the combination therapy.

### SUMMARY OF THE INVENTION

The invention provides for TLR inhibitor prodrug (“TLR Prodrug”) compositions comprising a TLR inhibitor agent, a lipid, and a biologically cleavable linker. In certain embodiments, nanocarriers comprising TLR Prodrug(s) are formulated for use as a delivery modality to treat human diseases such as cancer, including solid tumor cancers as well as other immunological disorders. In certain embodiments, the nanocarriers comprise a lipid-bilayer capable of being incorporated into a drug delivery vehicle (i.e., a liposome). In a further embodiment, the nanocarrier comprises a solid-lipid nanoparticle (“SLNP”). In a further preferred embodiment, the liposome comprises cholesterol hemisuccinate (“CHEMS”). In a further preferred embodiment, the liposome of the invention comprises Stearic Acid. In a further preferred embodiment, the liposome of the invention comprises a Stearic Acid derivative that is not cleavable.

In a further embodiment, an TLR Prodrug of the disclosure comprises an TR12-Prodrug.

In a further embodiment, an TLR Prodrug of the disclosure comprises an TR13-Prodrug.

In a further embodiment, the invention comprises methods of delivering a TLR inhibitor to a tumor comprising (i) synthesizing a TLR prodrug; (ii) formulating a TLR prodrug of the invention in a nanocarrier of the invention; and (iii) administering the nanocarrier to a patient.

In another embodiment, the invention comprises methods of delivering a TLR inhibitor with one or more additional immune modulating agent to a tumor comprising (i) synthesizing a TLR prodrug; (ii) co-formulating a TLR prodrug of the invention in a nanocarrier with one or more additional immune modulating agents of the invention; and (iii) administering the nanocarrier to a patient.

In another embodiment, the immune modulating agents comprise agonists of other TLRs, immunogenic-cell death (ICD) inducing chemotherapeutics, PD-1/PD-L1 antagonists, IDO antagonists, STING agonists, CTLA4 inhibitors, iNKT cell agonists, TGFβ inhibitors, A2aR inhibitors, and/or prodrugs thereof.

In another embodiment, the present disclosure teaches methods of synthesizing TLR prodrugs.

In another embodiment, the present disclosure teaches methods of synthesizing TR12 prodrugs.

In another embodiment, the present disclosure teaches methods of synthesizing TL13 prodrugs.

In another embodiment, the present disclosure teaches methods of formulating TLR prodrugs within nanocarriers, including but not limited to liposomes.

In another embodiment, the present disclosure teaches methods of formulating TR12 prodrugs within nanocarriers, including but not limited to liposomes.

In another embodiment, the present disclosure teaches methods of formulating TR13 prodrugs within nanocarriers, including but not limited to liposomes.

In another embodiment, the present disclosure teaches methods of formulating an TR12 Prodrug within nanocarriers, including but not limited to SLNPs.

In another embodiment, the present disclosure teaches methods of formulating an TR13 Prodrug within nanocarriers, including but not limited to SLNPs.

In another embodiment, the present disclosure teaches methods of treating cancer(s), immunological disorders and other diseases in humans using nanocarriers of the present disclosure.

### BRIEF DESCRIPTION OF THE FIGURES

FIG. 1. General Chemical Synthesis for TR12.

FIG. 2. Chemical Synthesis for TR12 & TR13 Prodrug Intermediate(s).

FIG. 3. General Chemical Synthesis for TR13.

FIG. 4. TLR Inhibitor Prodrug Synthesis Schema with Carboxylic Acid Functionality.

FIG. 5. TLR Inhibitor Prodrug Synthesis Schema with Alcohol Functionality.

FIG. 6. TLR Inhibitor Prodrug Synthesis Schema with Secondary Amine, Amide, or Aniline Functionality.

FIG. 7. Characterization of SLNP-TR12 Solid-Lipid Nanocarrier.

FIG. 8. Characterization of SLNP-TR12 Solid-Lipid Nanocarrier (Zeta Potential).

FIG. 9. Characterization of SLNP-IC1-TR12 Solid-Lipid Nanocarrier (@8:1, NTI 121).

FIG. 10. Characterization of SLNP-IC1-TR12 Solid-Lipid Nanocarrier (@8:1, NTI 121) (Zeta Potential).

FIG. 11. Characterization of SLNP-IC1-TR12 Solid-Lipid Nanocarrier (@16:1).

FIG. 12. Characterization of SLNP-IC1-TR12 Solid-Lipid Nanocarrier (@16:1) (Zeta Potential).

FIG. 13. Characterization of SLNP-AR5-TR12 Solid-Lipid Nanocarrier.

FIG. 14. Characterization of SLNP-AR5-TR12 Solid-Lipid Nanocarrier (Zeta Potential).

FIG. 15. Characterization of SLNP-TR12-NTI-47C Solid-Lipid Nanocarrier.

FIG. 16. Characterization of SLNP-TR12-NTI-47C Solid-Lipid Nanocarrier (Zeta Potential).

FIG. 17. Tumor Inhibition of SLNP-TR12 as a Single Agent in EMT6 Murine Breast Cancer Cells.

FIG. 18. Tumor Inhibition of SLNP-TR12 in various doses compared against SLNP-TR5 in various doses in EMT6 Murine Breast Cancer Cells.

FIG. 19. Tumor Inhibition of SLNP-TR12 in Combination with SLNP-IC1 in EMT6 Murine Breast Cancer Cells.

FIG. 20. Tumor Inhibition of SLNP-TR12 in Combination with SLNP-IC1 in EMT6 Murine Breast Cancer Cells.

FIG. 21. In Vitro Validation of TR12 Prodrug in Solid-Lipid Nanoparticle (“SLNP”) Form Mechanism of Action.

FIG. 22. In Vitro Validation of SLNP-TR12 versus SLNP-TR5 Mechanism of Action.

FIG. 23. In Vitro Validation of SLNP-TR12 Specificity to Toll-Like Receptor 7. FIG. 23(A) shows activity of SLNP-TR5 and SLNP-TR12 in RAW-Blue cells. FIG. 23(B) shows specificity of SLNP-TR12 to TLR-7 in HEK-Blue cells.

FIG. 24. Ex Vivo Validation of SLNP-TR12 Activity in Murine Splenocytes.

FIG. 25. Ex Vivo Validation of SLNP-TR12 Activity in human PBMCs.

FIG. 26. In-vivo Validation of SLNP-TR12 Efficacy in EMT-6 Tumor Model.

FIG. 27. In-vivo Validation of SLNP-TR12 Efficacy in 4T-1 Tumor Model.

FIG. 28. In-vivo Validation of Multiple Doses of TR12 Prodrug Alone and in Combination with IC1 Prodrug Efficacy in EMT-6 Tumor Model.

FIG. 29. Maximum Tolerated Dose (MTD) of Doxorubicin Prodrug alone and in Combination with SLNP-TR12 in Balb/c Mouse Model.

FIG. 30. In-vivo Validation of Doxorubicin Prodrug Efficacy in B16F10 Melanoma Tumor Model.

FIG. 31. In-vivo Validation of Doxorubicin Prodrug Efficacy in MPC11 Multiple Myeloma Tumor Model.

FIG. 32. In-vivo Validation of Doxorubicin Prodrug Efficacy in Neuro2A Neuroblastoma Tumor Model.

FIG. 33. In-vivo Validation of Doxorubicin Prodrug Efficacy in CT26 Colon Tumor Model.

FIG. 34. In-vivo Validation of Doxorubicin Prodrug Efficacy in MC38 Colon Tumor Model.

FIG. 35. In-vivo Validation of Doxorubicin Prodrug Efficacy in Renca Kidney Tumor Model.

FIG. 36. In-vivo Validation of Doxorubicin Prodrug Efficacy in H22 Liver Tumor Model.

FIG. 37. In-vivo Validation of Doxorubicin Prodrug Efficacy in Hepa1-6 Liver Tumor Model.

FIG. 38. In-vivo Validation of Doxorubicin Prodrug Efficacy in LLC1 Lung Tumor Model.

FIG. 39. In-vivo Validation of Doxorubicin Prodrug Efficacy in KLN205 Lung Tumor Model.

FIG. 40. In-vivo Validation of Doxorubicin Prodrug Efficacy in B16BL6 Melanoma Tumor Model.

FIG. 41. In-vivo Validation of Doxorubicin Prodrug Efficacy in Pan02.03 Pancreatic Tumor Model.

FIG. 42. In-vivo Validation of Doxorubicin Prodrug Efficacy in RM-1 Prostate Tumor Model.

FIG. 43. In-vivo Validation of Doxorubicin Prodrug Efficacy in BMT2 Bladder Tumor Model.

FIG. 44. In-vivo Validation of Doxorubicin Prodrug Efficacy in Clone M-3 Melanoma Tumor Model.

FIG. 45. In-vivo Validation of Doxorubicin Prodrug Efficacy in 4T1 Breast Orthotopic Tumor Model.

FIG. 46. In-vivo Validation of Multiple TR12 Prodrug(s) Alone or in Combination with IC1 Prodrug Efficacy in CT26 Tumor Model.

FIG. 47. In-vivo Validation of Multiple TR12 Prodrug(s) Alone or in Combination with Multiple Doses of IC1 Prodrug Efficacy in B16F10 Tumor Model.

FIG. 48. In-vivo Validation of TR12 Prodrug(s) Alone or in Combination with IC1 Prodrug Efficacy in EMT-6 Tumor Model.

FIG. 49. Ex-vivo Validation of CD47 Ability to Block Cellular Uptake. FIG. 49(A). Show lower cytokine secretion levels in groups treated with SLNP-TR12-47c or SLNP-TR12/47d in PBMCs. FIG. 49(B). Show lower cytokine secretion levels in groups treated with SLNP-TR12-47c or SLNP-TR12/47d in Splenocytes. FIG. 49(C). Show lower cytokine secretion levels in groups treated with SLNP-TR12-47c or SLNP-TR12/47d in additional Splenocytes.

FIG. 50. Ex-vivo Validation of Immunomodulatory Effects of SLNP-TR12 on Tumor-Infiltrating Lymphocytes in Balb/C Mice. FIG. 50(A). Shows the MFI. FIG. 50(B). Shows results in NK cells. FIG. 50(C). Shows results in total T-cells. FIG. 50(D). Shows results in cytotoxic T-cells.

FIG. 51. In-vitro Validation of CD47 Ability to Block Cellular Uptake.

### DETAILED DESCRIPTION OF THE INVENTION

### Outline of Sections

I.) Definitions
II.) Prodrugs
III.) Drug Moieties
IV.) Lipids
V.) Linkage Unit(s) (“LU”)
VI.) Nanocarriers
VII.) Liposomes
VIII.) Pharmaceutical Formulation
IX.) Combination Therapy
X.) Methods of Delivering Liposomes Comprising Prodrugs to a Cell
XI.) Methods of Treating Cancer(s) and Other Immunological Disorder(s)
XII.) KITS/Articles of Manufacture

I. Definitions:

Unless otherwise defined, all terms of art, notations and other scientific terms or terminology used herein are intended to have the meanings commonly understood by those of skill in the art to which this invention pertains unless the context clearly indicates otherwise. In some cases, terms with commonly understood meanings are defined herein for clarity and/or for ready reference, and the inclusion of such definitions herein should not necessarily be construed to represent a substantial difference over what is generally understood in the art.

When a trade name is used herein, reference to the trade name also refers to the product formulation, the generic drug, and the active pharmaceutical ingredient(s) of the trade name product, unless otherwise indicated by context.

As used herein, the term “about”, when referring to a value or to an amount of size (i.e., diameter), weight, concentration or percentage is meant to encompass variations of in one example±20% or ±10%, in another example ±5%, in another example±1%, and in still another example±0.1% from the specified amount, as such variations are appropriate to perform the disclosed methods.

As used herein, the term “and/or” when used in the context of a listing of entities, refers to the entities being present singly or in combination. Thus, for example, the phrase “A, B, C, and/or D” includes A, B, C, and D individually, but also includes any and all combinations and sub combinations of A, B, C, and D.

Numerical ranges recited herein by endpoints include all numbers and fractions subsumed within that range (e.g., 1 to 5 includes, but is not limited to, 1, 1 .5, 2, 2.75, 3, 3.90, 4, and 5).

As used herein, the phrase “consisting essentially of” limits the scope of a claim to the specified materials or steps, plus those that do not materially affect the basic and novel characteristic(s) of the claimed subject matter.

The terms “advanced cancer”, “locally advanced cancer”, “advanced disease” and “locally advanced disease” mean cancers that have extended through the relevant tissue capsule and are meant to include stage C disease under the American Urological Association (AUA) system, stage C1-C2 disease under the Whitmore-Jewett system, and stage T3-T4 and N+disease under the TNM (tumor, node, metastasis) system. In general, surgery is not recommended for patients with locally advanced disease and these patients have substantially less favorable outcomes compared to patients having clinically localized (organ-confined) cancer.

As used herein the term “alkyl” can refer to C₁-C₂₀ inclusive, linear (i.e. , “straight-chain”), branched, or cyclic, saturated, or at least partially and in some cases unsaturated (i.e. , alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl, or propyl, is attached to a linear alkyl chain. “Lower alkyl” refers to an alkyl group having 1 to about 8 carbon atoms (i.e., a C₁-C₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁-C₈ straight-chain alkyls. In other embodiments, “alkyl” refers, in particular, to Ci-₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. In some embodiments, there can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered aromatic and heteroaromatic rings. The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl. Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

“Heteroaryl” as used herein refers to an aryl group that contains one or more non-carbon atoms (e.g., O, N, S, Se, etc.) in the backbone of a ring structure. Nitrogen-containing heteroaryl moieties include, but are not limited to, pyridine, imidazole, benzimidazole, pyrazole, pyrazine, triazine, pyrimidine, and the like.

The terms “anticancer drug”, “chemotherapeutic”, and “anticancer prodrug” refer to drugs (i.e., chemical compounds) or prodrugs known to, or suspected of being able to treat a cancer (i.e., to kill cancer cells, prohibit proliferation of cancer cells, or treat a symptom related to cancer). In some embodiments, the term “chemotherapeutic” as used herein refers to a non-PS molecule that is used to treat cancer and/or that has cytotoxic ability. More traditional or conventional chemotherapeutic agents can be described by mechanism of action or by chemical compound class, and can include, but are not limited to, alkylating agents (e.g., melphalan), anthracyclines (e.g., doxorubicin), cytoskeletal disruptors (e.g., paclitaxel), epothilones, histone deacetylase inhibitors (e.g., vorinostat), inhibitors of topoisomerase I or II (e.g., irinotecan or etoposide), kinase inhibitors (e.g., bortezomib), nucleotide analogs or precursors thereof (e.g., methotrexate), peptide antibiotics (e.g., bleomycin), platinum based agents (e.g., cisplatin or oxaliplatin), retinoids (e.g., tretinoin), and vinka alkaloids (e.g., vinblastine).

“Aralkyl” refers to an -alkyl-aryl group, optionally wherein the alkyl and/or aryl moiety is substituted.

“Alkylene” refers to a straight or branched bivalent aliphatic hydrocarbon group having from 1 to about 20 carbon atoms, e.g., 1 , 2, 3, 4, 5, 6, 7, 8, 9, 10, 11, 12, 13, 14, 15, 16, 17, 18, 19, or 20 carbon atoms. The alkylene group can be straight, branched, or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyi”), wherein the nitrogen substituent is alkyl as previously described. Exemplary alkylene groups include methylene (—CH₂—); ethylene (—CH₂—CH₂—); propylene (—(CH₂)3—); cyclohexylene (—C₆H₁₀—); —CH═CH—CH═CH—; —CH═CH—CH₂—; —(CH₂)_{q}—N(R)—(CH₂)—, wherein each of q is an integer from 0 to about 20, e.g., 0, 1 , 2, 3, 4, 5, 6, 7, 8, 9, 10, 11, 12, 13, 14, 15, 16, 17, 18, 19, or 20, and R is hydrogen or lower alkyl; methylenedioxyl (—0—CH₂—0—); and ethylenedioxyl (—0—(CH₂)₂—0—). An alkylene group can have about 2 to about 3 carbon atoms and can further have 6-20 carbons.

The term “arylene” refers to a bivalent aromatic group, e.g., a bivalent phenyl or napthyl group. The arylene group can optionally be substituted with one or more aryl group substituents and/or include one or more heteroatoms.

The term “amino” refers to the group —N(R)₂ wherein each R is independently H, alkyl, substituted alkyl, aryl, substituted aryl, aralkyl, or substituted aralkyl. The terms “aminoalkyl” and “alkylamino” can refer to the group —N(R)₂ wherein each R is H, alkyl or substituted alkyl, and wherein at least one R is alkyl or substituted alkyl. “Arylamine” and “aminoaryl” refer to the group —N(R)₂ wherein each R is H, aryl, or substituted aryl, and wherein at least one R is aryl or substituted aryl, e.g., aniline (i.e., —NHC₆H₅).

A “bioreactive nanomaterial” refers to an engineered biomaterial that induces or catalyzes a biological response. In certain embodiments the nanomaterial induces a response by virtue of one or more properties selected from the group consisting of composition, size, shape, aspect ratio, dissolution, electronic, redox, surface display, surface coating, hydrophobic, hydrophilic, an atomically thin nanosheet, or functionalized surface groups” to catalyze the biological response at various nano/bio interfaces. In certain embodiments the bioreactive nanomaterial has the ability to inhibit TLR-1 biological responses in cells (e.g., in tumor cells) and/or as well as activating the innate immune system through delivery of “danger signal” and adjuvant effects.

“Bulk” (a.k.a. Drug Substance) means the drug substance or the drug product which has not been filled into final containers for distribution. Final formulated bulk generally refers to drug product which is formulated and being stored or held prior to filling. Drug substance may be stored or held as “bulk” or “concentrated bulk” prior to formulation into drug product.

The terms “carboxylate” and “carboxylic acid” can refer to the groups —C(═O)O⁻ and —C(═O)OH, respectively. The term “carboxyl” can also refer to the —C(═O)OH group.

The terms “conjugate” and “conjugated” as used herein can refer to the attachment (e.g., the covalent attachment) of two or more components (e.g., chemical compounds, polymers, biomolecule, particles, etc.) to one another. In some embodiments, a conjugate can comprise monovalent moieties derived from two different chemical compounds covalently linked via a bivalent linker moiety (e.g., an optionally substituted alkylene or arylene). In some embodiments, the linker can contain one or more biodegradable bond, such that one or more bonds in the linker can be broken when the prodrug is exposed to a particular physiological environment or enzyme (for example, esterases).

The term “compound” refers to and encompasses the chemical compound (e.g. a prodrug) itself as well as, whether explicitly stated or not, and unless the context makes clear that the following are to be excluded: amorphous and crystalline forms of the compound, including polymorphic forms, where these forms may be part of a mixture or in isolation; free acid and free base forms of the compound, which are typically the forms shown in the structures provided herein; isomers of the compound, which refers to optical isomers, and tautomeric isomers, where optical isomers include enantiomers and diastereomers, chiral isomers and non-chiral isomers, and the optical isomers include isolated optical isomers as well as mixtures of optical isomers including racemic and non-racemic mixtures; where an isomer may be in isolated form or in a mixture with one or more other isomers; isotopes of the compound, including deuterium- and tritium-containing compounds, and including compounds containing radioisotopes, including therapeutically- and diagnostically-effective radioisotopes; multimeric forms of the compound, including dimeric, trimeric, etc. forms; salts of the compound, preferably pharmaceutically acceptable salts, including acid addition salts and base addition salts, including salts having organic counterions and inorganic counterions, and including zwitterionic forms, where if a compound is associated with two or more counterions, the two or more counterions may be the same or different; and solvates of the compound, including hemisolvates, monosolvates, disolvates, etc., including organic solvates and inorganic solvates, said inorganic solvates including hydrates; where if a compound is associated with two or more solvent molecules, the two or more solvent molecules may be the same or different. In some instances, reference made herein to a compound of the invention will include an explicit reference to one or of the above forms, e.g., salts and/or solvates; however, this reference is for emphasis only, and is not to be construed as excluding other of the above forms as identified above.

“Drug product” means a final formulation that contains an active drug ingredient (i.e., liposomes containing TLR inhibitor prodrugs) generally, but not necessarily, in association with inactive ingredients. The term also includes a finished dosage form that does not contain an active ingredient but is intended to be used as a placebo.

The term “disulfide” can refer to the —S—S— group.

The term “empty vesicle” means an unloaded lipid vesicle by itself.

The term “ester” as used herein means a chemical compound derived from acid (organic or inorganic) in which at least one -OH hydroxyl group is replaced by an —O-alkyl (alkoxy) or O-Aryl (aryloxy) group.

The term “esterase” as used herein is a hydrolase enzyme that splits esters into an acid and an alcohol.

“Excipient” means an inactive substance used as a carrier for the active ingredients in a drug such as vaccines. Excipients are also sometimes used to bulk up formulations with very potent active ingredients, to allow for convenient and accurate dosage. Examples of excipients include but are not limited to, anti-adherents, binders, coatings, disintegrants, fillers, dilutants, flavors, colors, lubricants, and preservatives.

The terms “halo”, “halide”, or “halogen” as used herein refer to fluoro, chloro, bromo, and iodo groups.

The terms “hydroxyl” and “hydroxy” refer to the —OH group.

The terms “inhibit” or “inhibition of” as used herein means to reduce by a measurable amount, or to prevent entirely.

The terms “individual” or “patient,” as used in the context of this disclosure can be used interchangeably.

As used herein, the term “ligand” refers generally to a species, such as a molecule or ion, which interacts, e.g., binds, in some way with another species. See MARTELL, A. E., and HANCOCK, R. P., Metal Complexes in Aqueous Solutions, Plenum: New York (1996), which is incorporated herein by reference in its entirety.

The term “lipid” as used herein refers to a class of naturally occurring (organic) compounds that are insoluble in polar solvents. In the context of the disclosure, a lipid refers to conventional lipids, phospholipids, cholesterol, chemically functionalized lipids for attachment of PEG and ligands, etc.

The term “lipid bilayer” or “LB” refers to any double layer of oriented amphipathic lipid molecules in which the hydrocarbon tails face inward to form a continuous non-polar phase.

The term(s) “liposome” or “lipid vesicle” or “vesicle” are used interchangeably to refer to an aqueous compartment enclosed by a lipid bilayer, as being conventionally defined (see, STRYER (1981) Biochemistry, 2d Edition, W. H. Freeman & Co., p. 213).

The term “mammal” refers to any organism classified as a mammal, including mice, rats, rabbits, dogs, cats, cows, horses, and humans. In one embodiment of the invention, the mammal is a mouse. In another embodiment of the invention, the mammal is a human.

The terms “mercapto” or “thiol” refer to the —SH group. The terms “metastatic cancer” and “metastatic disease” mean cancers that have spread to regional lymph nodes or to distant sites and are meant to include stage D disease under the AUA system and stage T×N×M+under the TNM system.

The terms “nanocarrier”, “nanoparticle,” and “nanoparticle drug carrier” are used interchangeably and refer to a nanostructure having an aqueous, solid, or polymeric interior core. In certain embodiments the nanocarrier comprises a lipid bilayer encasing (or surrounding or enveloping) the porous particle core. In certain embodiments the nanocarrier is a liposome, lipid nanoparticle (“LNP”) or a solid-lipid nanoparticle (“SLNP”).

The terms “nanoscale particle,” “nanomaterial,” “nanocarrier”, and “nanoparticle” refer to a structure having at least one region with a dimension (e.g., length, width, diameter, etc.) of less than about 1,000 nm. In some embodiments, the dimension is smaller (e.g., less than about 500 nm, less than about 250 nm, less than about 200 nm, less than about 150 nm, less than about 125 nm, less than about 100 nm, less than about 80 nm, less than about 70 nm, less than about 60 nm, less than about 50 nm, less than about 40 nm, less than about 30 nm or even less than about 20 nm). In some embodiments, the dimension is between about 20 nm and about 250 nm (e.g., about 20, 30, 40, 50, 60, 70, 80, 90, 100, 110, 120, 130, 140, 150, 160, 170, 180, 190, 200, 210, 220, 230, 240, or 250 nm).

The term “nanovesicle” refers to a “lipid vesicle” having a diameter (or population of vesicles having a mean diameter) ranging from about 20 nm, or from about 30 nm, or from about 40 nm, or from about 50 nm up to about 500 nm, or up to about 400 nm, or up to about 300 nm, or up to about 200 nm, or up to about 150 nm, or up to about 100 nm, or up to about 80 nm. In certain embodiments a nanovesicle has a diameter ranging from about 40 nm up to about 80 nm, or from about 50 nm up to about 70 nm.

“Pharmaceutically acceptable” refers to a non-toxic, inert, and/or composition that is physiologically compatible with humans or other mammals.

“Pharmaceutical formulation” means the process in which different chemical substances are combined to a pure drug substance to produce a final drug product.

The term “phosphonate” refers to the —P(═O)(OR)₂ group, wherein each R can be independently H, alkyl, aralkyl, aryl, or a negative charge (i.e., wherein effectively there is no R group present to bond to the oxygen atom, resulting in the presence of an unshared pair of electrons on the oxygen atom). Thus, stated another way, each R can be present or absent, and when present is selected from H, alkyl, aralkyl, or aryl.

The term “phosphate” refers to the —OP(═O)(OR′)₂ group, where R′ is H or a negative charge.

The term “prodrug” means a medication or compound that, after administration, is metabolized into a pharmacologically active drug. For the purposes of this disclosure, a prodrug of the invention comprises three (3) components: (i) a drug moiety; (ii) a lipid moiety; and (iii) a linkage unit (“LU”).

The term “TLR prodrug” means a prodrug of the inventions wherein the drug moiety comprises a TLR agonist.

The term “pyrolipid” refers to a conjugate of a lipid and a porphyrin, porphyrin derivative, or porphyrin analog. In some embodiments, the pyrolipid can comprise a lipid conjugate wherein a porphyrin or a derivative or analog thereof is covalently attached to a lipid side chain. See, for example U.S. Patent Application Publication No. 2014/0127763.

As used herein, the terms “specific”, “specifically binds” and “binds specifically” refer to the selective binding of nanocarrier of the invention to the target TLR-1 or related family member.

The term “supported lipid bilayer” means a lipid bilayer enclosing a porous particle core. This definition as set forth in the disclosure is denoted because the lipid bilayer is located on the surface and supported by a porous particle core. In certain embodiments, the lipid bilayer can have a thickness ranging from about 6 nm to about 7 nm which includes a 3-4 nm thickness of the hydrophobic core, plus the hydrated hydrophilic head group layers (each about 0.9 nm) plus two partially hydrated regions of about 0.3 nm each. In various embodiments, the lipid bilayer surrounding the liposome comprises a continuous bilayer or substantially continuous bilayer that effectively envelops and seals the TLR inhibitor.

The term “thioalkyl” can refer to the group -SR, wherein R is selected from H, alkyl, substituted alkyl, aralkyl, substituted aralkyl, aryl, and substituted aryl. Similarly, the terms “thioaralkyl” and “thioaryl” refer to -SR groups wherein R is aralkyl and aryl, respectively.

As used herein “to treat” or “therapeutic” and grammatically related terms, refer to any improvement of any consequence of disease, such as prolonged survival, less morbidity, and/or a lessening of side effects which are the byproducts of an alternative therapeutic modality; as is readily appreciated in the art, full eradication of disease is a preferred but albeit not a requirement for a treatment act.

The term “therapeutically effective amount” refers to the amount of active prodrug, nano-encapsulated prodrug, or pharmaceutical agent that elicits the biological or medicinal response in a tissue, system, animal, individual or human.

The term “unsupported lipid bilayer” means an uncoated lipid bilayer in a lipid vesicle or liposome.

II.) Prodrugs

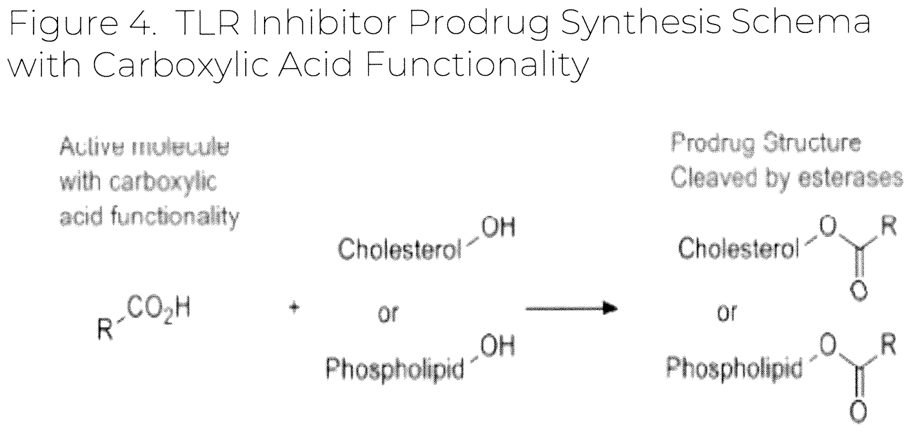

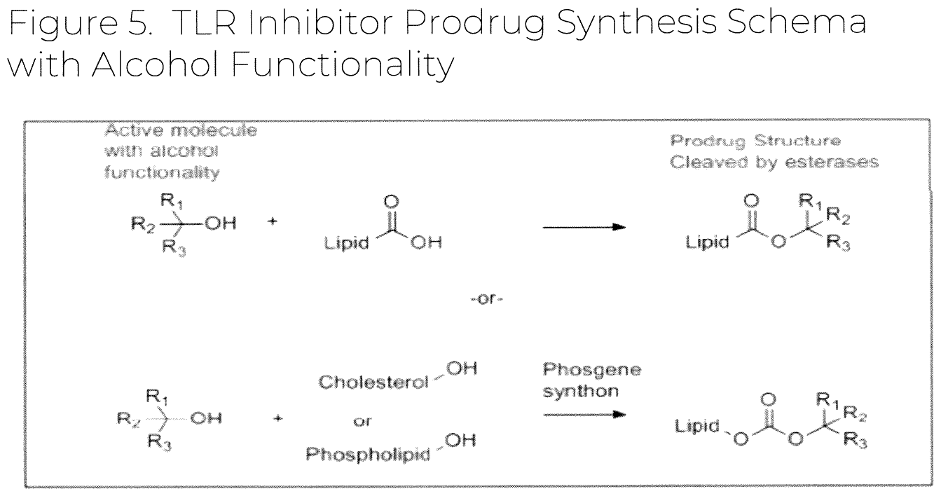

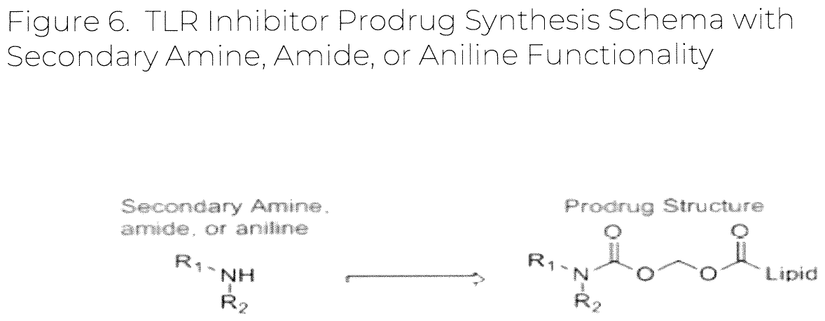

As shown in the present disclosure and for the purposes of this invention, a suitable prodrug is formed by conjugating a drug moiety of the invention (See, section entitled Drug Moieties) to a lipid moiety of the invention (See, section entitled Lipids) via an LU (See, section entitled Linkage Units) of the present disclosure. For the purposes of this disclosure, formation of a TLR prodrug can utilize several strategies. (See, for example, FIG. 4, FIG. 5, and FIG. 6).

Accordingly, in some embodiments, the prodrug is a drug-lipid moiety comprising a TLR inhibitor of the disclosure.

In a further embodiment, the prodrug is a drug-lipid moiety comprising a TLR inhibitor of the disclosure, wherein the TLR inhibitor inhibits TLR1/2.

In a further embodiment, the prodrug is a drug-lipid moiety comprising a TLR inhibitor of the disclosure, wherein the TLR inhibitor inhibits TLR4.

In a further embodiment, the prodrug is a drug-lipid moiety comprising a TLR inhibitor of the disclosure, wherein the TLR inhibitor inhibits TLR7.

In a further embodiment, the prodrug is a drug-lipid moiety comprising a TLR inhibitor of the disclosure, wherein the TLR inhibitor inhibits TLR8.

In a further embodiment, the prodrug is a drug-lipid moiety comprising a TLR inhibitor of the disclosure, wherein the TLR inhibitor inhibits TLR7/8.

In a further embodiment, the prodrug is a drug-lipid moiety comprising a TLR inhibitor of the disclosure, wherein the TLR inhibitor inhibits TLR1/2, and wherein the prodrug comprises a prodrug from Formula I.

In one embodiment, the prodrug comprises the following chemical structure denoted Formula I:

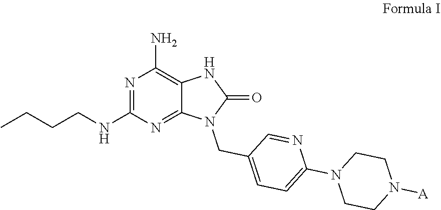

Wherein, in exemplary embodiments of FORMULA I:

A=

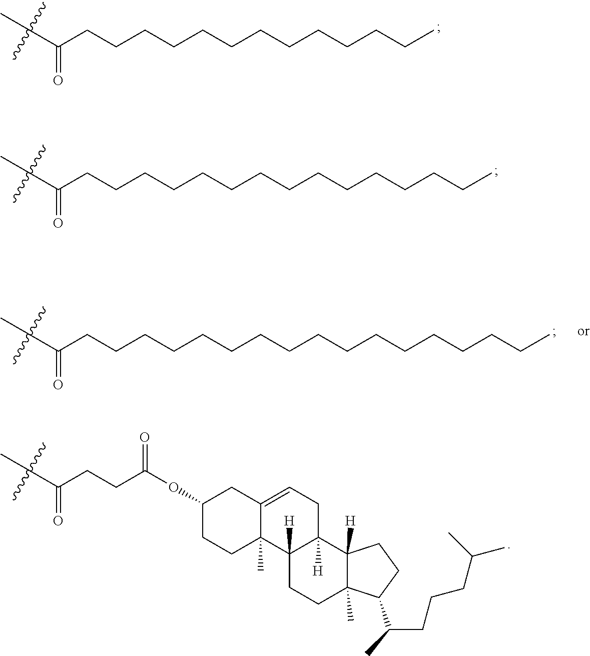

Thus, in one embodiment, the prodrug is a drug-lipid moiety comprising a TLR inhibitor of FORMULA I.

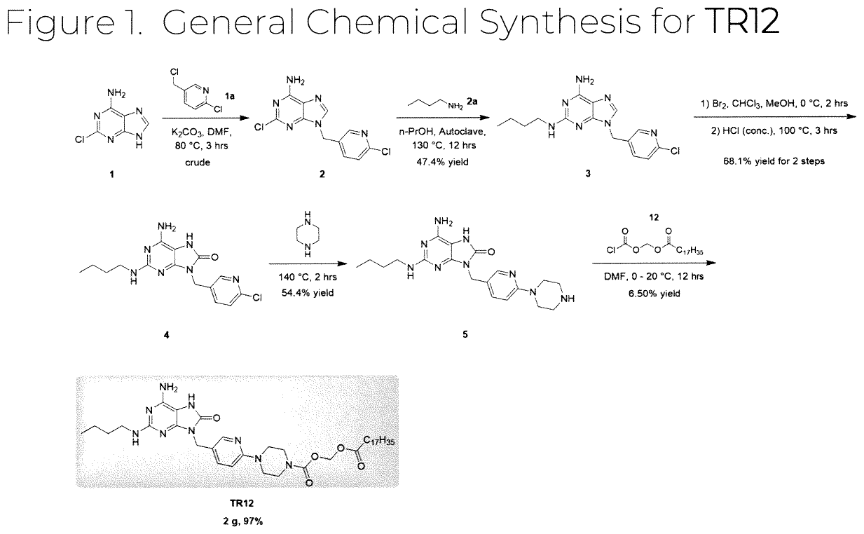

In one embodiment, the prodrug is a drug-lipid moiety comprising a TLR inhibitor set forth in FIG. 1.

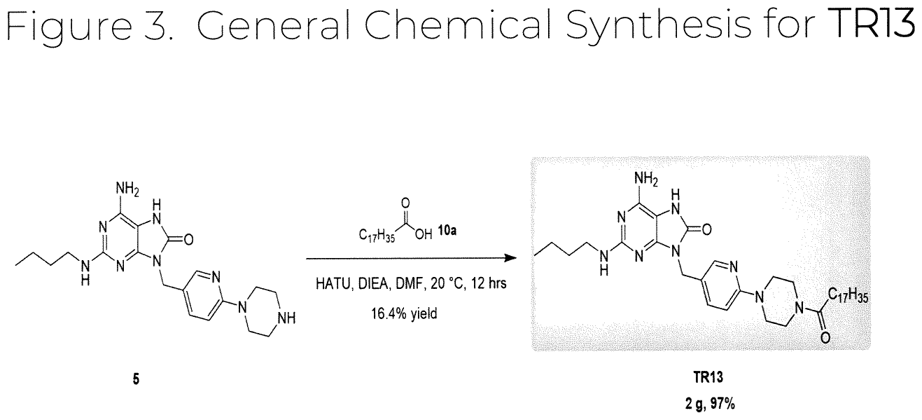

In one embodiment, the prodrug is a drug-lipid moiety comprising a TLR inhibitor set forth in FIG. 3.

In a further embodiment, the TLR prodrug is a drug-lipid moiety comprising a lipid of the disclosure.

In a further embodiment, the TLR prodrug is a drug-lipid moiety whereby the lipid is CHEMS.

In a further embodiment, the TLR prodrug is a drug-lipid moiety whereby the lipid is Stearic Acid.

In a further embodiment, the TLR prodrug is a drug-lipid moiety whereby the lipid is a Stearic Acid derivative that is non-cleavable.

In a further embodiment, the TLR prodrug is a drug-lipid moiety comprising a LU of the disclosure.

In a further embodiment, the TLR prodrug is a drug-lipid moiety whereby the LU is a hydromethylcarbamate linker.

In a further embodiment, the prodrug is a drug-lipid moiety comprising a TLR inhibitor of the invention, wherein the TLR inhibitor comprises the chemical composition(s) TR12 and/or TR13.

In a further embodiment, the prodrug is a drug-lipid moiety comprising a TLR inhibitor of the invention, wherein the TLR inhibitor comprises TR12 and/or TR13 and further comprises CHEMS.

In a further embodiment, the prodrug is a drug-lipid moiety comprising a TLR inhibitor of the invention, wherein the TLR inhibitor comprises TR12 and/or TR13 and further comprises Stearic Acid.

In a further embodiment, the prodrug is a drug-lipid moiety comprising a TLR inhibitor of the invention, wherein the TLR inhibitor comprises TR13 and further comprises a non-cleavable Stearic Acid derivative.

In a further embodiment, the prodrug is a drug-lipid moiety comprising a TLR inhibitor of the invention, wherein the TLR inhibitor comprises TR12 and further comprises CHEMS and whereby the LU is a hydromethylcarbamate linker.

In a further embodiment, the prodrug is a drug-lipid moiety comprising a TLR inhibitor of the invention, wherein the TLR inhibitor comprises TR12 and further comprises Stearic Acid and whereby the LU is a hydromethylcarbamate linker.

In a further embodiment, the prodrug is a drug-lipid moiety comprising a TLR inhibitor of the inventions, wherein the TLR inhibitor comprises TR12 and further comprises Stearic Acid having the following structure:

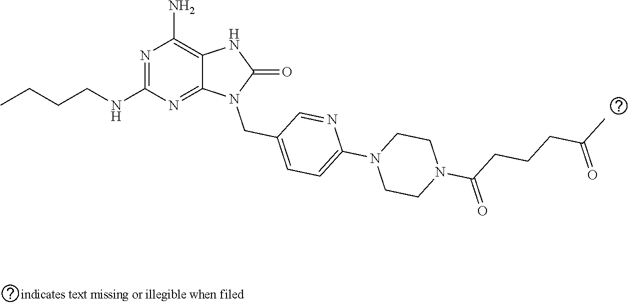 [SMILES: CCCCNc1nc(N)c2[nH]c(=O)n(Cc3ccc(N4CCN(C(=O)CCCC(C)=O)CC4)nc3)c2n1]

In a further embodiment, the prodrug is a drug-lipid moiety comprising a TLR inhibitor of the invention, wherein the TLR inhibitor comprises TR13 and further comprises a non-cleavable Stearic Acid derivative having the following structure:

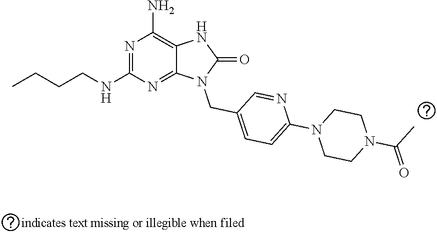 [SMILES: CCCCNc1nc(N)c2[nH]c(=O)n(Cc3ccc(N4CCN(C(C)=O)CC4)nc3)c2n1]

In a further embodiment, the prodrug is a drug-lipid moiety comprising a TLR inhibitor of the invention, wherein the TLR inhibitor comprises TR12 and further comprises a lipid of the disclosure having the following chemical formula:

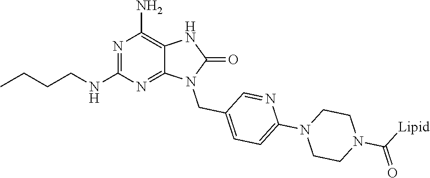

### (“TR12-Prodrug”).

In additional embodiments of the disclosure the subject matter provides a TLR inhibitor prodrug comprising a lipid-conjugated therapeutic agent parent drug. In some embodiments, the prodrug comprises: (a) a monovalent drug moiety, (b) a monovalent lipid moiety, and (c) a bivalent linker moiety comprising a linkage unit that will degrade in vivo, such as a disulfide bond, wherein the monovalent drug moiety and the monovalent lipid moiety are linked (e.g., covalently linked) through the linker. The monovalent drug moiety and the monovalent lipid moieties can be monovalent derivatives of a chemical compound and a lipid, respectively. For instance, the monovalent derivative can be a deprotonated derivative of a chemical compound or lipid that comprises a hydroxyl, thiol, amino, or carboxylic acid group.

In further embodiments of the disclosure the subject matter provides a TLR inhibitor prodrug comprising a lipid-conjugated therapeutic agent parent drug. In some embodiments, the prodrug comprises: (a) a bivalent drug moiety, (b) a bivalent lipid moiety, and (c) a bivalent linker moiety comprising a linkage that will degrade in vivo, wherein the bivalent drug moiety and the bivalent lipid moiety are linked (e.g., covalently linked) through the linker. The bivalent drug moiety and the bivalent lipid moieties can be bivalent derivatives of a chemical compound and a lipid, respectively. For instance, the bivalent derivative can be a deprotonated derivative of a chemical compound or lipid that comprises a hydroxyl, thiol, amino, or carboxylic acid group.

One of ordinary skill in the art will appreciate and be enabled to make variations and modifications to the disclosed embodiment without altering the function and purpose of the invention disclosed herein. Such variations and modifications are intended within the scope of the present disclosure.

III.) Drug Moieties

Another aspect of the invention provides for novel TLR prodrug compound(s) with the following formula(s) denoted TR12 and TR13.

One of skill in the art will appreciate that a compound is useful as a TLR inhibitor (e.g., inhibits TLR, for example TLR1/2, TLR4, TLR7, TLR8 and/or TLR7/8). By way of brief background, TLR-1 (CD281) recognizes pathogen-associated molecular pattern with a specificity for gram-positive bacteria. TLR-1 is found on the epithelial cell layer that lines the small and large intestine and is an important player in the management of the gut microbiota and detection of pathogens. It is also found on the surface of macrophages and neutrophils. TLR1 recognizes peptidoglycan and (triacyl) lipopeptides in concert with TLR2 (as a heterodimer) and has been clearly shown to interact with TLR2. See, FARHAT, et. al., J. Leukoc. Biol. 83(3):692-701 (2007) and JIN, et. al., Cell. 130(6):1071-1082 (2007).

TLR2 (CD282) is a protein that in humans is encoded by the TLR2 gene. TLR2 is a membrane protein which is expressed on the surface of certain cells and recognizes foreign substances and passes on appropriate signals to the cells of the immune system. TLR2 is expressed most abundantly in peripheral blood leukocytes and mediates host response to Gram-positive bacteria and yeast via stimulation of NF-κB. See, BARRELLO, et. al., Int. J. Immun. & Pharm. 24(3):549-556 (2011). TLR2 resides on the plasma membrane where it responds to lipid-containing PAMPs such as lipoteichoic acid and di- and tri-acylated cysteine-containing lipopeptides. It does this by forming dimeric complexes with either TLR 1 or TLR6 on the plasma membrane. See, BOTOS, et. al., Structure 19(4):447-459 (2011).

TLR4 (CD284) is another member of the TLR family. Its activation leads to an intracellular signaling pathway NF-κB and inflammatory cytokine production which is responsible for activating the innate immune system. It is most well-known for recognizing lipopolysaccharide (LPS), a component present in many Gram-negative bacteria (e.g., Neisseria spp.) and select Gram-positive bacteria. Its ligands also include several viral proteins, polysaccharide, and a variety of endogenous proteins such as low-density lipoprotein, beta-defensins, and heat shock protein. See, BRUBAKER, et. al., Annual Rev. of Immun. 33:257-290 (2015). TLR4 signaling responds to signals by forming a complex using an extracellular leucine-rich repeat domain (LRR) and an intracellular toll/interleukin-1 receptor (TIR) domain. LPS stimulation induces a series of interactions with several accessory proteins which form the TLR4 complex on the cell surface. LPS recognition is initiated by an LPS binding to an LBP protein. The conformational changes of the TLR4 induce the recruitment of intracellular adaptor proteins containing the TIR domain which is necessary to activate the downstream signaling pathway. LU, et. at, Cytokine 42(2):145-151 (2008). TLR4 is capable of activating MAPK and NF-κB pathways, implicating possible direct role of cell-autonomous TLR4 signaling in regulation of carcinogenesis, in particular, through increased proliferation of tumor cells, apoptosis inhibition and metastasis.

TLR7 is another member of the TLR family. TLR7 recognizes single-stranded RNA in endosomes, which is a common feature of viral genomes which are internalized by macrophages and dendritic cells. TLR7 recognizes single-stranded RNA of viruses such as HIV and HCV. See, HEIL, et. al., Science 303(5663):1526-1529 (2004). TLR7 can recognize GU-rich single-stranded RNA. Id. However, the presence of GU-rich sequences in the single-stranded RNA is not sufficient to stimulate TLR7. TLR7 has been shown to play a significant role in the pathogenesis of autoimmune disorders such as Systemic Lupus Erythematosus (SLE) as well as in the regulation of antiviral immunity. In addition, due to their ability to induce robust production of anti-cancer cytokines such as interleukin-12, TLR7 agonists have been investigated for cancer immunotherapy.

TLR8 is another member of the family and is a protein that has been designated as CD288. TLR8 is predominantly expressed in lung and peripheral blood leukocytes, and lies in close proximity to another family member, TLR7. TLR8 is an endosomal receptor that recognizes single stranded RNA (ssRNA), and can recognize ssRNA viruses such as influenza, Sendai, and Coxsackie B viruses. TLR8 binding to the viral RNA recruits MyD88 and leads to activation of the transcription factor NF-kB and an anti-viral response. See, ZHANG, et. al., Sci. Rep., 6, 29447; doi:10:1038/srep29447 (2016).

Based on the foregoing, the present disclosure describes a class of TLR inhibitors.

In one embodiment, the class of TLR inhibitors inhibit TLR1/2.

In one embodiment, the class of TLR inhibitors inhibit TLR7.

In one embodiment, a drug moiety of the disclosure comprises a compound with the following chemical structure (denoted TR12):

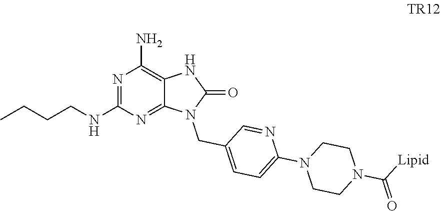

One of ordinary skill in the art will appreciate and be enabled to make variations and modifications to the disclosed embodiment without altering the function and purpose of the invention disclosed herein. Such variations and modifications are intended within the scope of the present disclosure.

IV.) Lipids

Generally speaking, and for the purposes of this disclosure, the term “lipid” is used in its broadest sense and comprises several sub-categories of lipids, including but not limited to, phospholipids/fatty acids. As it is appreciated by one of skill in the art, a phospholipid represents a class of lipids that are a major component of all cell membranes. Phospholipids can form lipid bilayers because of their amphiphilic characteristic. The structure of the phospholipid molecule generally consists of two hydrophobic fatty acid “tails” and a hydrophilic “head” consisting of a phosphate group that can be modified with simple organic molecules such as choline, ethanolamine, or serine. These two components are usually joined together by a glycerol molecule. A representative list of phospholipids/fatty acid(s) of the invention are set forth in Table III.

By way of brief background, at the most fundamental level, the properties of a liposome depend upon the subtle physicochemical interactions among the various lipid species in its composition. Individual lipids can be combined to form a myriad of superstructures including bilayers, and bilayer properties can be tuned to modulate drug release and membrane stability. In a simplified bilayer model acyl chain length dictates bilayer thickness and phase transition temperature (Tm), acyl chain saturation controls bilayer fluidity, and headgroup interactions impact inter- and intra-lipid molecular forces. Liposome behavior can be adjusted by incorporating synthetic lipids such as lipid prodrugs, fusogenic lipids and functionalizable lipids into the bilayer. See, KOHLI, et. al., J. Control Release, 0:pp. 274-287 (Sep. 28, 2014).

In one embodiment of the present disclosure, a TLR prodrug comprises a monovalent lipid moiety.

In one embodiment, a TLR prodrug comprises a bivalent lipid moiety.

In one embodiment, the lipid comprises a cholesterol with the following chemical structure:

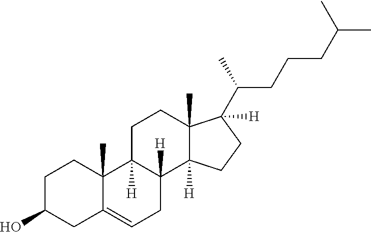

In one embodiment, the lipid comprises a DPPG with the following chemical structure:

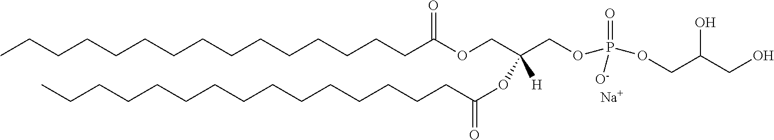 [SMILES: CCCCCCCCCCCCCCCC(=O)OC[C@H](COP(=O)([O-])OCC(O)CO)OC(=O)CCCCCCCCCCCCCCC ; [Na+]]

In one embodiment, the lipid comprises a DMPG with the following chemical structure:

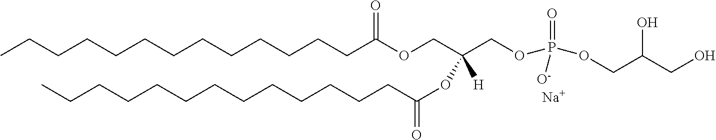 [SMILES: CCCCCCCCCCCCCC(=O)OC[C@H](COP(=O)([O-])OCC(O)CO)OC(=O)CCCCCCCCCCCCC ; [Na+]]

In one embodiment, the lipid comprises a Lyso PC with the following chemical structure:

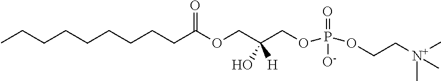 [SMILES: CCCCCCCCCC(=O)OC[C@@H](O)COP(=O)([O-])OCC[N+](C)(C)C]

In one embodiment, the lipid comprises a (Δ9-Cis) PG.

In one embodiment, the lipid comprises a Soy Lyso PC with the following chemical structure:

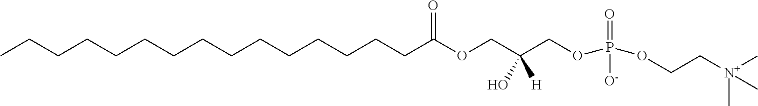 [SMILES: CCCCCCCCCCCCCCCC(=O)OC[C@@H](O)COP(=O)([O-])OCC[N+](C)(C)C]

In one embodiment, the lipid comprises a PG with the following chemical structure:

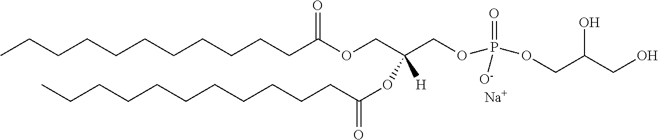 [SMILES: CCCCCCCCCCCC(=O)OC[C@H](COP(=O)([O-])OCC(O)CO)OC(=O)CCCCCCCCCCC ; [Na+]]

In one embodiment, the lipid comprises a C16 PEG2000 Ceramde with the following chemical structure:

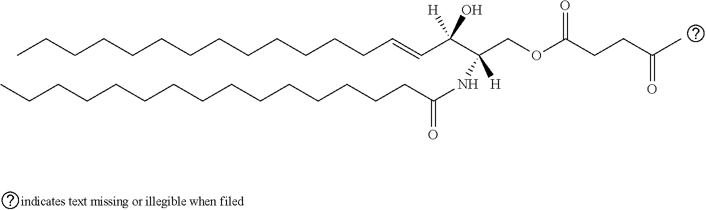 [SMILES: CCCCCCCCCCCCC/C=C/[C@@H](O)[C@H](COC(=O)CCC(C)=O)NC(=O)CCCCCCCCCCCCCCC]

In one embodiment, the lipid comprises a cholesterol hemisuccinate (“CHEMS”) with the following chemical structure:

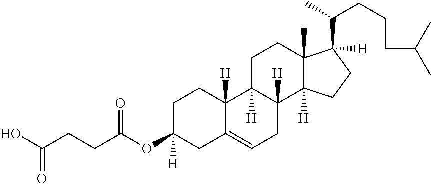

In one embodiment, the lipid comprises a class of lipids having the following chemical structure denoted Formula II:

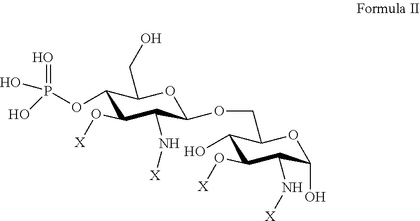

Wherein, in exemplary embodiments of FORMULA II:

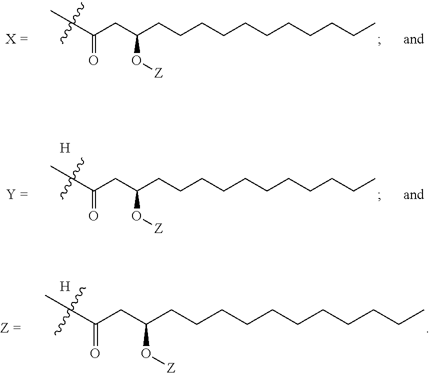

In one embodiment, the lipid comprises a class of lipids having the following chemical structure denoted Formula III:

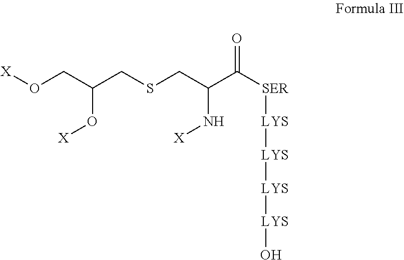

Wherein, in exemplary embodiments of FORMULA III:

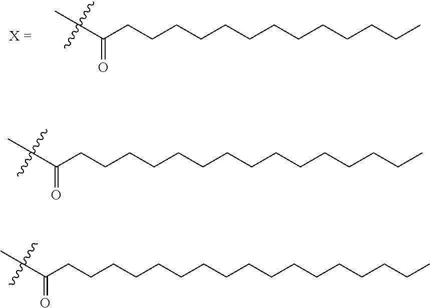 [SMILES: *C(=O)CCCCCCCCCCCCC ; *C(=O)CCCCCCCCCCCCCCC ; *C(=O)CCCCCCCCCCCCCCCCC]

In a further embodiment, a lipid moiety of the disclosure comprises a class of invariant natural killer T (iNKT) cells.

In a further embodiment, a lipid moiety of the disclosure comprises Alpha-galactosylceramide (α-GalCer).

By way of reference, a further list of the chemical formulas and abbreviation(s) of the lipids disclosed herein is set forth in Table I.

In an additional embodiment, the lipid comprises a phospholipid/fatty acid disclosed herein and set forth in Table III.

In a further embodiment, the lipid comprises a Stearic acid.

In a further embodiment, the lipid comprises a non-cleavable Stearic Acid derivative.

In addition, the TLR prodrugs and/or liposome(s) of the disclosure may comprise one or more helper lipids which are also referred to herein as “helper lipid components”. The helper lipid components are preferably selected from the group comprising phospholipids and steroids. Phospholipids are preferably di- and monoester of the phosphoric acid. Preferred members of the phospholipids are phosphoglycerides and sphingolipids. Steroids, as used herein, are naturally occurring and synthetic compounds based on the partially hydrogenated cyclopenta[a]phenanthrene. Preferably, the steroids contain 21 to 30 C atoms. A particularly preferred steroid is cholesterol.

It is to be noted that although not wishing to be bound by any theory, due to the particular mol percentages of the helper lipid(s) contained in the lipid compositions according to the present invention, which helper lipid can be either a PEG-free helper lipid or in particular a PEG-containing helper lipid, surprising effects can be realized, more particularly if the content of any of this kind of helper lipid is contained within the concentration range specified herein.

In a further aspect of the present invention, lipid compositions which are preferably present as lipoplexes or liposomes, preferably show a neutral or overall anionic charge. The anionic lipid is preferably any neutral or anionic lipid described herein. The lipid composition comprises in a preferred embodiment any helper lipid or helper lipid combination as well as any TLR inhibitor as described herein. In a further embodiment the composition according to the present invention containing nucleic acid(s) forms lipoplexes. In a preferred embodiment the term lipoplexes as used herein refers to a composition composed of neutral or anionic lipid, neutral helper lipid and TLR inhibitor of the invention. For reference into the usage of helper lipids in the art, see, by way of example, U.S. Patent Application Publication 2011/0178164; OJEDA, et. al., Int. J. of Pharmaceutics (March 2016); DABKOWSKA, et. al., J. R. Soc. Interface 9, pp. 548-561 (2012); and MOCHIZUKI, et. al., Biochimica et. Biophysica Acta, 1828, pp. 412-418 (2013).

In a preferred embodiment, the helper lipids of the invention comprise the helper lipids set forth in Table II.

In one embodiment, a TLR prodrug comprises a lipid of the invention, wherein the lipid is CHEMS and wherein the drug moiety is TR12.

In one embodiment, a TLR prodrug comprises a lipid of the invention, vitherein the lipid is CHEMS and wherein the drug moiety is TR12, further comprising a LU and wherein the LU is a hydromethylcarbamate linker.

In one embodiment, a TLR prodrug comprises a lipid of the invention, wherein the lipid is CHEMS and wherein the drug moiety is TR12, further csomprising a LU and wherein the LU is a hydromethylcarbamate linker, further comprising a helper lipid component, wherein the helper lipid component comprises a helper lipid of Table II.

In one embodiment, a TLR prodrug comprises a lipid of the invention, wherein the lipid is CHEMS and wherein the drug moiety is TR12 and wherein the CHEMS is monovalent.

In one embodiment, a TLR prodrug comprises a lipid of the invention, wherein the lipid is Stearic Acid and wherein the drug moiety is TR12.

In one embodiment, a TLR prodrug comprises a lipid of the invention, wherein the lipid is Stearic Acid and wherein the drug moiety is TR12 and wherein the Stearic Acid is monovalent.

In one embodiment, a TLR prodrug comprises a lipid of the invention, wherein the lipid is Stearic Acid and wherein the drug moiety is TR12, further comprising a LU and wherein the LU is a hydromethylcarbamate linker.

In one embodiment, a TLR prodrug comprises a lipid of the invention, wherein the lipid is Stearic Acid and wherein the chemical composition is TR12, further comprising a LU and wherein the LU is a hydromethylcarbamate linker, further comprising a helper lipid component, wherein the helper lipid component comprises a helper lipid of Table II.

One of ordinary skill in the art will appreciate and be enabled to make variations and modifications to the disclosed embodiment without altering the function and purpose of the invention disclosed herein. Such variations and modifications are intended within the scope of the present disclosure.

V.) Linkage Unit(s) (“LU”)

In some embodiments, the presently disclosed subject matter provides prodrugs comprising drug-lipid conjugates that include biodegradable linkages, such as esters, thioesters, and other linkers known in the art.

Exemplary embodiments of ester chemistry are set forth herein:

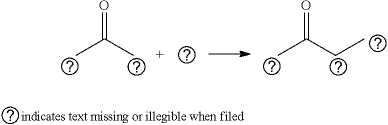

In some embodiments, the prodrug is a drug-lipid conjugate, whereby the drug-lipid conjugate is cleaved by an esterase.

In one embodiment, a prodrug of the invention comprises a LU via a secondary amine, amide, or aniline using the following schema:

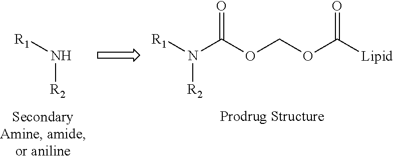

An exemplary synthesis is as follows:

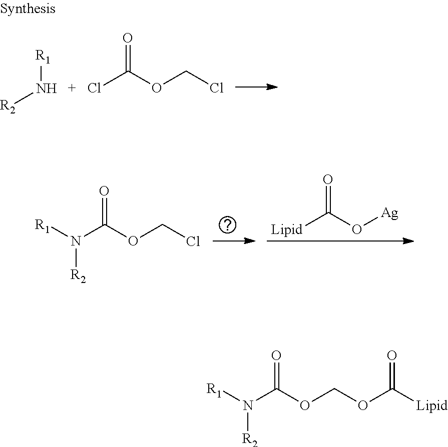

Cleavage of the prodrug structure comprising a secondary amine, amide, or aniline is obtained via esterase hydrolysis of the secondary amine, amide, or aniline prodrug under the following exemplary synthesis:

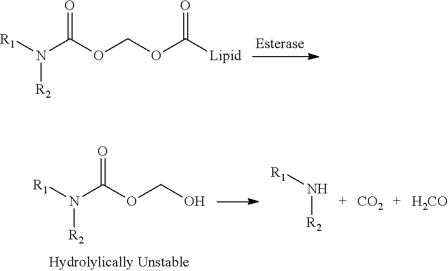

### Wherein:

R₁ and R₂ can be and molecule which connects a N via a C.

In one embodiment, the secondary amide nitrogen of the TR12 drug moiety is conjugated to CHEMS via a hydromethylcarbamate linker.

In one embodiment, the secondary amide nitrogen of the TR12 drug moiety is conjugated to Stearic Acid via a hydromethylcarbamate linker.

One of ordinary skill in the art will appreciate and be enabled to make variations and modifications to the disclosed embodiment without altering the function and purpose of the invention disclosed herein. Such variations and modifications are intended within the scope of the present disclosure.

VI.) Nanocarrier(s) Generally speaking, and for the purposes of this disclosure nanocarrier(s) are within the scope of the invention. A nanocarrier is nanomaterial being used as a transport module for another substance, such as a drug. Commonly used nanocarriers include micelles, polymers, carbon-based materials, liposomes, solid-lipid nanoparticles, and other substances. Because of their small size, nanocarriers can deliver drugs to otherwise inaccessible sites around the body. Nanocarriers can include polymer conjugates, polymeric nanoparticles, lipid-based carriers, dendrimers, carbon nanotubes, and gold nanoparticles. Lipid-based carders include both liposomes and micelles. In certain embodiments the nanocarrier is a liposome, lipid nanoparticle (“LNP”) or a solid-lipid nanoparticle (“SLNP”).

In addition, nanocarriers are useful in the drug delivery process because they can deliver drugs to site-specific targets, allowing drugs to be delivered in certain organs or cells but not in others. Site-specificity poses a major therapeutic benefit since it prevents drugs from being delivered to the wrong places. In addition, nanocarriers show promise for use in chemotherapy because they can help decrease the adverse, broader-scale toxicity of chemotherapy on healthy, fast-growing cells around the body. Since chemotherapy drugs can be extremely toxic to human cells, it is important that they are delivered to the tumor without being released into other parts of the body.

Generally speaking, there are four (4) methods in which nanocarriers can deliver drugs and they include passive targeting, active targeting, pH specificity, and temperature specificity.

Passive targeting refers to a nanocarrier's ability to travel down a tumor's vascular system, become trapped, and accumulate in the tumor. This accumulation is caused by the enhanced permeability and retention effect. The leaky vasculature of a tumor is the network of blood vessels that form in a tumor, which contain many small pores. These pores allow nanocarriers in, but also contain many bends that allow the nanocarriers to become trapped. As more nanocarriers become trapped, the drug accumulates at the tumor site. This accumulation causes large doses of the drug to be delivered directly to the tumor site.

Active targeting involves the incorporation of targeting modules such as ligands or antibodies on the surface of nanocarriers that are specific to certain types of cells around the body. Generally, nanocarriers have a high surface-area to volume ratio allowing for multiple ligands to be incorporated on their surfaces.

Additionally, certain nanocarriers will only release the drugs they contain in specific pH ranges. pH specificity also allows nanocarriers to deliver drugs directly to a tumor site. This is due to the fact that tumors are generally more acidic than normal human cells, with a pH around 6.8. Normal tissue has a pH of around 7.4. Thus, nanocarriers that only release drugs at certain pH ranges can therefore be used to release the drug only within acidic tumor environments. High acidic environments cause the drug to be released due to the acidic environment degrading the structure of the nanocarrier. Generally, these nanocarriers will not release drugs in neutral or basic environments, effectively targeting the acidic environments of tumors while leaving normal body cells untouched. This pH sensitivity can also be induced in micelle systems by adding copolymer chains to micelles that have been determined to act in a pH independent manor. See, WU, et. al., Biomaterials, 34(4):1213-1222 (2012). These micelle-polymer complexes also help to prevent cancer cells from developing multi-drug resistance. The low pH environment triggers a quick release of the micelle polymers, causing a majority of the drug to be released at once, rather than gradually like other drug treatments.

Additionally, some nanocarriers have also been shown to deliver drugs more effectively at certain temperatures. Since tumor temperatures are generally higher than temperatures throughout the rest of the body, around 40° C., this temperature gradient helps act as safeguard for tumor-specific site delivery. See, REZAEI, et. al., Polymer, 53(16):3485-3497 (2012).

As disclosed herein, lipid-based nanocarriers, such as liposomes are within the scope of this invention. Lipid-based nanoparticles (LBNPs or LNPs) such as liposomes, solid lipid nanoparticles (SLN) and nanostructured lipid carriers (NLC) can transport hydrophobic and hydrophilic molecules, display exceptionally low or no toxicity, and increase the time of drug action by means of a prolonged half-life and a controlled release of the drug. Lipid nanoparticles can include chemical modifications to avoid the detection by the immune system (gangliosides or polyethylene glycol (PEG)) or to improve the solubility of the drug. In addition, they can be prepared in formulations sensitive to the pH in order to promote drug release in an acid environment and can also be associated with small molecules or antibodies that recognize tumor cells or their receptors (such as folic acid (FoA)). Nanodrugs can also be used in combination with other therapeutic strategies to improve the response of patients. See, GARCIA-PINEL, et. al., Nanomaterials 9(639) (2019).

In various embodiments silicasome drug carriers described herein comprise a porous silica (or other material) nanoparticle (e.g., a silica body having a surface and defining a plurality of pores that are suitable to receive molecules therein) coated with a lipid bilayer. The fact that the nanoparticle is referred to as a silica nanoparticle does not preclude materials other than silica from also being incorporated within the silica nanoparticle. In some embodiments, the silica nanoparticle may be substantially spherical with a plurality of pore openings through the surface providing access to the pores. However, in various embodiments the silica nanoparticle can have shapes other than substantially spherical shapes. Thus, for example, in certain embodiments the silica nanoparticle can be substantially ovoid, rod-shaped, a substantially regular polygon, an irregular polygon, and the like.

Generally, the silica nanoparticle comprises a silica body that defines an outer surface between the pore openings, as well as side walls within the pores. The pores can extend through the silica body to another pore opening, or a pore can extend only partially through the silica body such that that it has a bottom surface of defined by the silica body.

In some embodiments, the silica body is mesoporous. In other embodiments, the silica body is microporous. As used herein, “mesoporous” means having pores with a diameter between about 2 nm and about 50 nm, while “microporous” means having pores with a diameter smaller than about 2 nm. In general, the pores may be of any size, but in typical embodiments are large enough to contain one or more therapeutic compounds therein. In such embodiments, the pores allow small molecules, for example, therapeutic compounds such as anticancer compounds to adhere or bind to the inside surface of the pores, and to be released from the silica body when used for therapeutic purposes. In some embodiments, the pores are substantially cylindrical.

In certain embodiments the nanoparticles comprise pores having pore diameters between about 1 nm and about 10 nm in diameter or between about 2 nm and about 8 nm. In certain embodiments the nanoparticles comprise pores having pore diameters between about 1 nm and about 6 nm, or between about 2 nm and about 5 nm. Other embodiments include particles having pore diameters less than 2.5 nm.

In other embodiments, the pore diameters are between 1.5 and 2.5 nm. Silica nanoparticles having other pore sizes may be prepared, for example, by using different surfactants or swelling agents during the preparation of the silica nanoparticles. In various embodiments the nanoparticles can include particles as large (e.g., average, or median diameter (or another characteristic dimension) as about 1000 nm. However, in various embodiments the nanoparticles are typically less than 500 nm or less than about 300 nm as, in general, particles larger than 300 nm may be less effective in entering living cells or blood vessel fenestrations. In certain embodiments the nanoparticles range in size from about 40 nm, or from about 50 nm, or from about 60 nm up to about 100 nm, or up to about 90 nm, or up to about 80 nm, or up to about 70 nm. In certain embodiments the nanoparticles range in size from about 60 nm to about 70 nm. Some embodiments include nanoparticles having an average maximum dimension between about 50 nm and about 1000 nm. Other embodiments include nanoparticles having an average maximum dimension between about 50 nm and about 500 nm. Other embodiments include nanoparticles having an average maximum dimension between about 50 nm and about 200 nm.

In some embodiments, the average maximum dimension is greater than about 20 nm, greater than about 30 nm, greater than 40 nm, or greater than about 50 nm. Other embodiments include nanoparticles having an average maximum dimension less than about 500 nm, less than about 300 nm, less than about 200 nm, less than about 100 nm or less than about 75 nm. As used herein, the size of the nanoparticle refers to the average or median size of the primary particles, as measured by transmission electron microscopy (TEM) or similar visualization techniques known in the art. Further examples of mesoporous silica nanoparticles include, but are not limited to, MCM-41, MCM-48, and SBA-15. See, KATIYARE, et. al., J. Chromotog. 1122(1-2):13-20 (2006).

Methods of making porous silica nanoparticles are well known to those of skill in the art. In certain embodiments mesoporous silica nanoparticle are synthesized by reacting tetraethyl orthosilicate (TEOS) with a template made of micellar rods. The result is a collection of nano-sized spheres or rods that are filled with a regular arrangement of pores. The template can then be removed by washing with a solvent adjusted to the proper pH (See, e.g., TREWYN et al. (2007) Chem. Eng. J. 137(1):23-29).

In certain embodiments mesoporous particles can also be synthesized using a simple sol-gel method (See, e.g., NANDIYANTO, et al. (2009) Microporous and Mesoporous Mat. 120(3):447-453). In certain embodiments tetraethyl orthosilicate can also be used with an additional polymer monomer as a template. In certain embodiments 3-mercaptopropyl)trimethoxysilane (MPTMS) is used instead of TEOS.

In certain embodiments the mesoporous silica nanoparticles are cores are synthesized by a modification of the sol/gel procedure described by MENG et. al. (2015) ACS Nemo, 9(4):3540-3557.

While the methods described herein have been demonstrated with respect to porous silica nanoparticles (e.g., mesoporous silica), it will be recognized by those skilled in the art that similar methods can be used with other porous nanoparticles. Numerous other mesoporous materials that can be used in drug delivery nanoparticles are known to those of skill in the art. For example, in certain embodiments mesoporous carbon nanoparticles could be utilized.

Mesoporous carbon nanoparticles are well known to those of skill in the art (See, e.g., HUANG et. al. (2016) Carbon, 101:135-142; ZHU et. al. (2014) Asian J. Pharm. Sci., 9(2):82-91; and the like).

Similarly, in certain embodiments, mesoporous polymeric particles can be utilized. The syntheses of highly ordered mesoporous polymers and carbon frameworks from organic-organic assembly of triblock copolymers with soluble, low-molecular-weight phenolic resin precursors (resols) by an evaporation induced self-assembly strategy have been reported by MENG, et. al. (2006) Chem. Mat. 6(18):4447-4464.

The nanoparticles described herein are illustrative and non-limiting. Using the teachings provided herein numerous other lipid bilayer coated nanoparticles will be available to one of skill in the art.

In one embodiment, the invention teaches nanocarriers which comprise TLR prodrugs.

In one embodiment, the invention teaches a nanocarrier comprising a liposome, wherein the lipid comprises CHEMS.

In one embodiment, the invention teaches a nanocarrier comprising a liposome, wherein the lipid comprises Stearic Acid.

In one embodiment, the invention teaches a nanocarrier comprising a liposome, wherein the lipid comprises non-cleavable Stearic Acid derivative.

In one embodiment, the invention teaches nanocarriers which comprise TLR prodrugs, wherein the TLR prodrug comprises TR12.

In one embodiment, the invention teaches a nanocarrier comprising a liposome, wherein the lipid comprises CHEMS and whereby the liposome further comprises a TLR prodrug.

In one embodiment, the invention teaches a nanocarrier comprising a liposome, wherein the lipid comprises CHEMS and whereby the liposome further comprises TR12.

In one embodiment, the invention teaches a nanocarrier comprising a liposome, wherein the lipid comprises Stearic Acid and whereby the liposome further comprises a TLR inhibitor.

In one embodiment, the invention teaches a nanocarrier comprising a liposome, wherein the lipid comprises Stearic Acid and whereby the liposome further comprises TR12.

In one embodiment, the invention teaches a nanocarrier comprising a liposome, wherein the lipid comprises Stearic Acid and whereby the liposome further comprises TR12 (denoted LNP-TR12).

In a further embodiment, the invention teaches a nanocarrier comprising a liposome, wherein the lipid comprises Stearic Acid and whereby the liposome further comprises TR12 and whereby the liposome is co-formulated with a A2aR antagonist, wherein the A2aR antagonist comprises an A2aR antagonist denoted AR5 (denoted LNP-TRI2-AR5).

In a further embodiment, the invention teaches a nanocarrier comprising a liposome, wherein the lipid comprises Stearic Acid and whereby the liposome further comprises TR12 and whereby the liposome is co-formulated with a TGFb inhibitor, wherein the TGFb inhibitor comprises a TGFb inhibitor denoted TB4 (denoted LNP-TRI2-TB4).

In a further embodiment, the invention teaches a nanocarrier comprising a liposome, wherein the lipid comprises Stearic Acid and whereby the liposome further comprises TR12 and whereby the liposome is co-formulated with a PD-1 antagonist, wherein the PD-1 antagonist comprises a PD-1 antagonist denoted PD3 (denoted LNP-TRI2-PD3).

In a further embodiment, the invention teaches a nanocarrier comprising a liposome, wherein the lipid comprises Stearic Acid and whereby the liposome further comprises TR12 and whereby the liposome is co-formulated with an IDO inhibitor, wherein the IDO inhibitor comprises an IDO inhibitor denoted ID3 (denoted LNP-TRI2-ID3).

In a preferred embodiment, the lipid particle comprises a solid-lipid nanoparticle (SLNP) comprising a liposome which comprises an TLR Prodrug.

In a preferred embodiment, the lipid particle comprises a solid-lipid nanoparticle (SLNP) comprising a liposome which comprises an TLR Prodrug, wherein the TLR Prodrug comprises TR12.

In a preferred embodiment, the lipid particle comprises a solid-lipid nanoparticle (SLNP) comprising a liposome which comprises an TLR Prodrug, wherein the TLR Prodrug comprises TR13.

In one embodiment, the invention teaches a nanocarrier comprising a solid-lipid nanoparticle (“SLNP”), wherein the solid-lipid nanoparticle comprises Stearic Acid and whereby the solid-lipid nanoparticle further comprises TR12 (denoted SLNP-TR12).

In a further embodiment, the invention teaches a nanocarrier comprising a SLNP, wherein the lipid comprises Stearic Acid and whereby the SLNP further comprises TR12 and whereby the SLNP is co-formulated with an A2aR antagonist, wherein the A2aR antagonist comprises a A2aR antagonist denoted AR5 (denoted SLNP-TR12-AR5).

In a further embodiment, the invention teaches a nanocarrier comprising a SLNP, wherein the lipid comprises Stearic Acid and whereby the SLNP further comprises TR12 and whereby the SLNP is co-formulated with an immunogenic cell death (“ICD”) inducing prodrug, wherein the ICD inducing prodrug comprises an ICD inducing prodrug denoted IC1 (denoted SLNP-TRI2-IC1).

In a further embodiment, the invention teaches a nanocarrier comprising a SLNP, wherein the lipid comprises Stearic Acid and whereby the SLNP further comprises TR12 and whereby the SLNP is co-formulated with an immunogenic cell death (“ICD”) inducing prodrug, wherein the ICD inducing prodrug comprises an ICD inducing prodrug denoted ICI and wherein the ratio is set forth as 8:1 (denoted SLNP-TR12-IC1 and/or NTI-121).

In a further embodiment, the invention teaches a nanocarrier comprising a SLNP, wherein the lipid comprises Stearic Acid and whereby the SLNP further comprises TR12 and whereby the SLNP is co-formulated with an immunogenic cell death (“ICD”) inducing prodrug, wherein the ICD inducing prodrug comprises an ICD inducing prodrug denoted ICI and wherein the ratio is set forth as 16:1 (denoted SLNP-TR12-IC1).

In a further embodiment, the invention teaches a nanocarrier comprising a SLNP, wherein the lipid comprises Stearic Acid and whereby the SLNP further comprises TR12 and whereby the SLNP is co-formulated with a custom peptide (GSGCERVIGTGWVRC) (SEQ ID NO: 1) conjugated to Palmitoyl (denoted SLNP-TR12-NTI-47C).

By using this peptide (CERVIGTGWVRC) (SEQ ID NO: 2) in this type of SLNP, it is contemplated that function-blocking peptide structurally mimics an epitope on CD47 and binds to SIRPα.

Furthermore, it is understood that the CD47 molecule is well known as a widely expressed cellular surface receptor activating the transduction of the “don't-eat-me” signal. Thereby, it can decrease the wanted uptake of the nanoparticies by macrophages and has the potential to stay in blood circulation for longer time.

In a further embodiment, the invention teaches a nanocarrier comprising a SLNP, wherein the lipid comprises Stearic Acid and whereby the SLNP further comprises TR12 and whereby the SLNP is co-formulated with a TGFb inhibitor, wherein the TGFb inhibitor comprises a TGFb inhibitor denoted TB4 (denoted SLNP-TR12-TB4).

In a further embodiment, the invention teaches a nanocarrier comprising a SLNP, wherein the lipid comprises Stearic Acid and whereby the SLNP further comprises TR12 and whereby the SLNP is co-formulated with a PD-1 antagonist, wherein the PD-1 antagonist comprises a PD-1 antagonist denoted PD3 (denoted SLNP-TR12-PD3).

In a further embodiment, the invention teaches a nanocarrier comprising a SLNP, wherein the lipid comprises Stearic Acid and whereby the SLNP further comprises TR12 and whereby the SLNP is co-formulated with an IDO inhibitor, wherein the IDO inhibitor comprises an IDO inhibitor denoted 103 (denoted SLNP-TR12-ID3).

In a further preferred embodiment, the solid-lipid nanoparticle of the invention comprises a composition having the following ratio(s):

| Constituent of the SLNP | Amount (% w/w) |
|---|---|
| Lipid 1 (lipid-prodrug) | 5-80 |
| Lipid 2 (lipid-prodrug) | 0-40 |
| Helper lipids | 0-80 |
| DSPE-PEG2000 | 0-10 |
| DMG-PEG2000 | 0-10 |
| CD47 mimicry protein | 0-50 |
| Stabilizer(s) | 0-20 |

In a further preferred embodiment, the solid-lipid nanoparticle of the invention comprises a composition having the following ratio(s):

| Constituent of the SLNP | Amount (% w/w) |
|---|---|
| Lipid 1 (lipid-prodrug) | 5-80 |
| Lipid 2 (lipid-prodrug) | 0-40 |
| Lipid 3 (Lipid-prodrug) | 0-30 |
| Helper lipids | 0-80 |
| DSPE-PEG2000 | 0-10 |
| DMG-PEG2000 | 0-10 |
| CD47 mimicry protein | 0-50 |
| Stabilizer(s) | 0-20 |

Whereby Lipid 1 comprises a TR12-Prodrug, wherein the lipid moiety comprises Stearic Acid and whereby the helper lipids are the helper lipids set forth in Table II and whereby the stabilizers are selected from the group consisting of polyvinyl alcohol (e.g., Moliwol 488), poloxamers (e.g., Pluronic F127), Tween 80, PEG400, and Kolliphor RH 40 and whereby Lipid 2 and Lipid 3 (lipid prodrug) comprises a lipid prodrug of the disclosure or a lipid prodrug selected from the group consisting of ID3, PD3, AR5, IC1, NTI-47C, and/or TB4 inhibitors (for examples ID3-STEA, PD3-STEA, AR5-STEA, TB4-STEA, IC1-STEA, NTI-47C-STEA, etc.), MPLA, and Telratolimod and whereby a CD47 mimicry protein comprises a peptide having the following sequence GSGCERVIGTGWVRC (SEQ ID NO: 1) conjugated to Palmitoly and/or Stearyl, etc. group(s). Functionally, the use of this peptide CERVIGTGWVRC (SEQ ID NO: 2) in this novel SLNP is the CD47 mimicry protein structurally mimics an epitope on CD47 and binds SIRPalpha.

One of ordinary skill in the art will appreciate and be enabled to make variations and modifications to the disclosed embodiment without altering the function and purpose of the invention disclosed herein. Such variations and modifications are intended within the scope of the present disclosure.

The scope of the disclosure teaches three (3) non-limiting possible treatment modalities using the formulated prodrugs of the invention. See, PCT Patent Publication No. WO2018/213631.

The first treatment modality involves combination of a TLR prodrug in combination with another therapeutic (e.g., another formulated prodrug which inhibits TLR (e.g., TLR1/2, TLR4, TLR7, TLR8, and/or TLR7/8), a chemotherapy agent (such as an ICD-inducing chemotherapy), etc.) into a single liposome that allows systemic (or local) biodistribution and drug delivery to tumor sites. The dual-delivery approach achieved synergistic enhancement of adaptive and innate immunity, leading to a significant improvement in animal survival. In certain embodiments the nanocarrier comprises a vesicle (i.e., a lipid bilayer enclosing a fluid).

A second treatment modality involves local delivery to a tumor or peri-tumoral region, of an agent that inhibits TLR in combination with a lipid (e.g., a liposome) that comprises an inhibitor of TLR (e.g., TLR1/2, TLR4, TLR7, TLR8, and/or TLR7/8). It is demonstrated that such local delivery of a TLR inhibitor in combination with a TLR prodrug induces cytotoxic tumor killing, and tumor shrinkage at the local site. These adaptive immune responses are accompanied by boosting of the innate immune system, as reflected by CRT expression, as well as the activation of a DC population, particularly well-suited for generating cytotoxic T cell responses.

A third treatment modality involves vaccination utilizing dying cancer cells {e.g., KPC cells) in which inhibition of TLR is induced ex vivo. It is discovered that such vaccination can generate a systemic immune response that can interfere with tumor growth at a remote site as well as allowing adoptive transfer to non-immune animals. One of skill in the art will appreciate and be enabled to perform methods the treatment modalities provided herein.

VII.) Liposomes

In one aspect, the presently disclosed subject matter is based on an approach for providing a prodrug of the disclosure (See, section entitled Prodrugs) suitable for incorporation into a nanocarrier comprising lipid coating layers to provide enhanced delivery of the corresponding prodrugs and for providing combination therapies including the prodrugs. The advantages for using prodrugs of the invention include the facilitation of controlled formulation into an LNP of the disclosure (e.g., a liposome). This allows the prodrug to be maintained in an inactive form during systemic circulation, which allows the liposome to release the active agent after engulfment by a cell, for example within a tumor.

In certain embodiments one or more TLR prodrugs (e.g., any one or more of the TLR prodrugs inhibitors taught in Formula I, and/or TR12 or TR13 (See, section entitled “Prodrugs”) are formulated a lipid moiety that forms a vesicle (e.g., a liposome) structure in aqueous solution or that can form a component of a lipid bilayer comprising a liposome.

In certain embodiments one or more TLR Lipid Moieties (e.g., any one or more of the TLR lipid moieties taught in Formula III (See, section entitled “Lipids”) are formulated and/or co-formulated within a vesicle (e.g., a liposome) structure in aqueous solution or that can form a component of a lipid bilayer comprising a liposome.

The liposomes can be used directly or provided as components in a combined formulation (e.g., in combination with another drug moiety, or lipid moiety, or therapeutic modality as disclosed herein).

In certain embodiments, the liposome that is formulated with the TLR prodrug comprises a lipid, PHGP, vitamin E, cholesterol, and/or a fatty acid.

In certain embodiments, the liposome that is formulated comprises a lipid moiety comprising TR12.

In certain embodiments, the liposome that is formulated comprises a lipid moiety comprising TR13.

In certain embodiments, the liposome that is formulated comprises a lipid moiety comprising Formula III.

In certain embodiments, the liposome that is formulated comprises a lipid moiety comprising Alpha-galactosylceramide (α-GalCer).

In one embodiment, the liposome comprises cholesterol.

In one embodiment, the liposome comprises DPPG.

In one embodiment, the liposome comprises DMPG.

In one embodiment, the liposome Lyso PC.

In one embodiment, the liposome (Δ9-Cis) PG.

In one embodiment, the liposome comprises Soy Lyso PC.

In one embodiment, the liposome comprises PG.

In one embodiment, the liposome comprises PA-PEG3-mannose.

In one embodiment, the liposome comprises C16 PEG2000 Ceramide.

In one embodiment, the liposome comprises MPLA.

In one embodiment, the liposome comprises 3-Deacly MPLA.

In one embodiment, the liposome comprises CHEMS.

In one embodiment, the liposome comprises Stearic Acid.

In one embodiment, the liposome comprises a phospholipid set forth in Table III.

In one embodiment, the liposome comprises TR12 and further comprises CHEMS and further comprises a LU wherein said LU is a hydromethylcarbamate linker.

In one embodiment, the liposome comprises TR12 and further comprises Stearic Acid and further comprises a LU wherein said LU is a hydromethylcarbamate linker.

In one embodiment, the liposome comprises TR12 and further comprises CHEMS and further comprises a LU wherein said LU is a hydromethylcarbamate linker and further comprises a helper lipid set forth in Table II.

In one embodiment, the liposome comprises TR12 and further comprises a Stearic Acid and further comprises a LU wherein said LU is a hydromethylcarbamate linker and further comprises a helper lipid set forth in Table II.

In one embodiment, the liposome comprises TR12.

In one embodiment, the liposome comprises TR13.

In one embodiment, the liposome of the disclosure comprises a TLR prodrug co-formulated with one or more additional immune modulating agents, whereby the immune modulating agents includes, but is not limited to, immunogenic-cell death inducing chemotherapeutics, IDO antagonists, sting agonists, CTLA4 inhibitors, PD-1 inhibitors, and/or prodrugs thereof.

In one embodiment, the liposome of the disclosure comprises a TLR prodrug co-formulated with one or more additional immune modulating agents, whereby the immune modulating agents includes, but is not limited to, neurokinin 1 (NK1) antagonists, and/or prodrugs thereof.

In one embodiment, the liposome of the disclosure comprises a TLR prodrug co-formulated with one or more additional immune modulating agents, whereby the immune modulating agents includes, but is not limited to, A2aR antagonists, and/or prodrugs thereof.

In a preferred embodiment, the liposome comprises a TLR prodrug co-formulated with an ICD-inducing Chemotherapeutic.

In a preferred embodiment, the liposome comprises a TLR prodrug co-formulated with an ICD-inducing Chemotherapeutic selected from the list: doxorubicin (DOX), mitoxantrone (MTO), Oxaliplatin (OXA), Cyclophosphamide (CP), Bortezomib, Carfilzimib, or Paclitaxel.

In a preferred embodiment, the liposome comprises a TLR prodrug co-formulated with a Toll Receptor TLR agonist/Prodrug.

In a preferred embodiment, the liposome comprises a TLR prodrug co-formulated with Toll Receptor (TLR) agonist/Prodrug selected from the list: Resiquimod (R848), Gardiquimod, 852A, DSR 6434, Telratolimod, CU-T12-9, monophosphoryl Lipid A (MPLA), 3D(6-acyl)-PHAD®, SMU127, Pam3CSK4, or 3D-PHAD®.

In a preferred embodiment, the liposome comprises a TLR prodrug co-formulated with an PD-1 inhibitor/Prodrug.

In a preferred embodiment, the liposome comprises a TLR prodrug co-formulated with an PD-1 inhibitor/Prodrug, selected from the list: AUNP12, CA-170, or BMS-986189 or prodrugs thereof.

In a preferred embodiment, the liposome comprises a TLR prodrug co-formulated with doxorubicin (DOX).

In a preferred embodiment, the liposome comprises a TLR prodrug co-formulated with mitoxantrone (MTO).

In a preferred embodiment, the liposome comprises a TLR prodrug co-formulated with doxorubicin (DOX) and an PD-1 prodrug.

In a preferred embodiment, the liposome comprises a TLR prodrug co-formulated with mitoxantrone (MTO) and a PD-1 prodrug.

In a preferred embodiment, the liposome comprises a TLR prodrug co-formulated with doxorubicin (DOX) and an IDO antagonist/prodrug.

In a preferred embodiment, the liposome comprises a TLR prodrug co-formulated with mitoxantrone (MTO) and an IDO antagonist/prodrug.

In a preferred embodiment, the liposome comprises a TLR prodrug co-formulated with doxorubicin (DOX) and a PD-1 prodrug and an IDO antagonist/prodrug.

In a preferred embodiment, the liposome comprises a TLR prodrug co-formulated with mitoxantrone (MTO) and a PD-1 prodrug and an IDO antagonist/prodrug.

In a preferred embodiment, the liposome comprises a TLR prodrug co-formulated with an IDO antagonist/prodrug.

In a preferred embodiment, the liposome comprises a TLR prodrug co-formulated with an IDO antagonist/prodrug.

In a preferred embodiment, the liposome comprises a TLR prodrug co-formulated with an IDO antagonist/prodrug and a PD-1 prodrug.

In a preferred embodiment, the liposome comprises a TLR prodrug co-formulated with an IDO antagonist/prodrug and a PD-1 prodrug.

In a preferred embodiment, the liposome comprises TR12 co-formulated with doxorubicin (DOX).

In a preferred embodiment, the liposome comprises TR12 co-formulated with mitoxantrone (MTO).

In a preferred embodiment, the liposome comprises TR12 co-formulated with doxorubicin (DOX) and/or an IDO prodrug and/or an IDO antagonist/prodrug.

In a preferred embodiment, the liposome comprises TR12 co-formulated with mitoxantrone (MTO) and/or an IDO prodrug and/or an IDO antagonist/prodrug.

In a preferred embodiment, the liposome comprises TR12 co-formulated with NK1.

In a preferred embodiment, the liposome comprises TR12 co-formulated with MTO.

In a preferred embodiment, the liposome comprises TR12 co-formulated with DOX and a A2aR prodrug.

In another preferred embodiment, the liposome comprises a solid-lipid nanoparticle (SLNP) comprising a liposome which comprises a TLR prodrug.

One of skill in the art will appreciate and understand that solubility is one of most common problems faced by the artisan in the drug development process. Chemical conjugation of a drug/anti-cancer agents via lipid molecules (i.e., lipid-based prodrugs) provides a platform to solve the problem of formulating the drugs in an aqueous suspension. The major advantages of delivering drug(s) with lipid conjugation (lipid-based prodrugs) lies on its ability to improve pharmacokinetics/half-life and targeted delivery.

With suitable selection of lipid molecules, lipid-based prodrug(s) can be integrated/formulated in a liposomal formulation using techniques known in the art, which has many more advantages over conventional drug delivery system. (KOHLI, et. al., J. Control Release, 0:pp 274-287 (Sep. 28, 2014); and GARCIA-PINEL, et. al., Nanomaterials 9:638 (2019). The advantage of combining lipid-prodrug with liposomes is twofold: (i) liposomes containing lipid-prodrug not only increase the solubility of the drug/prodrug itself, but (ii) also have the ability to encapsulate multiple drugs (both hydrophilic and lipophilic) (see, section entitled nanocarriers).

For the purposes of this disclosure, the major advantage of liposome formulations are as follows:
i) biocompatibility/biodegradability and no general toxicity of the liposome's formulations;
ii) flexibility and manipulation of size and surface charge depending on the required purpose. Liposome formulation(s), for the purposes of this disclosure, can have a size range of 40-150 nm in diameter and a surface charge in the range of −40 to +40 mV; and
iii) Liposomes of the invention have either a single or multiple lipid-prodrugs as the constituent lipid portion of the liposome(s). Additionally, multiple drugs (e.g., that work in different mechanism of action) and with different solubility profile (hydrophilic or lipophilic) can be formulated (either in the lipid bilayers or in the hydrophilic core) in these liposomes.

As one of ordinary skill in the art will appreciate, all methods of making liposomes involve four (4) basic stages:
(i) Drying down lipids from organic solvent;
(ii) Dispersing the lipid in aqueous solution;
(iii) Purifying the resultant liposome; and
(iv) Analyzing the final product.

### See, AKBARZADEH, et. al., Nanoscale Research Letters, 8:102 (2013).

Another aspect of the invention discloses liposomal encapsulation technology (LET) which is a delivery technique used to transmit drugs. LET is a method of generating sub-microscopic foams called liposomes, which encapsulate numerous materials. These ‘liposomes’ form a barrier around their contents, which is resistant to enzymes in the mouth and stomach, alkaline solutions, digestive juices, bile salts, and intestinal flora that are generated in the human body, as well as free radicals. The contents of the liposomes are, therefore, protected from oxidation and degradation. This protective phospholipid shield or barrier remains undamaged until the contents of the liposome are delivered to the exact target gland, organ, or system where the contents will be utilized (See, section entitled nanocarriers).

In one embodiment, liposome(s) of the disclosure are synthesized using a plurality of different ratios of TLR prodrugs, TLR lipid moieties, lipids, and/or lipid-prodrugs. As disclosed herein, the TLR prodrugs may comprise helper lipids as disclosed herein (See, for example Table II).

In one embodiment, liposome(s) of the disclosure are synthesized using a plurality of different ratios of TLR prodrugs, TLR lipid moieties, lipids, and/or lipid-prodrugs. As disclosed herein, the TLR prodrugs may further comprise DSPE-PEGs.

In a preferred embodiment, the liposomes of the invention comprise a composition having the followinn ratio(s):

| Constituent of the Liposome | Amount (% w/w) |
|---|---|
| Lipid 1 (lipid-prodrug) | 5-60 |
| Lipid 2 (lipid-prodrug) | 0-60 |
| Helper lipids | 0-50 |
| DSPEG-PEG 2000 | 2-5 |

In a further preferred embodiment, the liposomes of the invention comprise a composition having the following ratio(s):

| Constituent of the Liposome | Amount (% w/w) |
|---|---|
| Lipid 1 (lipid-prodrug) | 5-60 |
| Helper lipids | 0-50 |
| DSPEG-PEG 2000 | 2-5 |

In a further preferred embodiment, the liposomes of the invention comprise a composition having the following ratio(s):

| Constituent of the Liposome | Amount (% w/w) |
|---|---|
| Lipid 1 (lipid-prodrug) | 5-60 |
| Helper lipids | 0-50 |
| DSPEG-PEG 2000 | 2-5 |

Whereby Lipid 1 comprises TR12 and CHEMS.

In a further preferred embodiment, the liposomes of the invention comprise a composition having the following ratio(s):

| Constituent of the Liposome | Amount (% w/w) |
|---|---|
| Lipid 1 (lipid-prodrug) | 5-60 |
| Helper lipids | 0-50 |
| DSPEG-PEG 2000 | 2-5 |

Whereby Lipid 1 comprises TR12 and Stearic Acid.

In a further preferred embodiment, the liposomes of the invention comprise a composition having the following ratio(s):

| Constituent of the Liposome | Amount (% w/w) |
|---|---|
| Lipid 1 (lipid-prodrug) | 5-60 |
| Helper lipids | 0-50 |
| DSPEG-PEG 2000 | 2-5 |

Whereby Lipid 1 comprises TR12 and a non-cleavable Stearic Acid derivative.

One of ordinary skill in the art will appreciate and be enabled to make variations and modifications to the disclosed embodiment without altering the function and purpose of the invention disclosed herein. Such variations and modifications are intended within the scope of the present disclosure.

VIII.) Pharmaceutical Formulation

As used herein, the term “drug” is synonymous with “pharmaceutical”. In certain embodiments, the liposome of the disclosure is fabricated to an encapsulated dosage form to and given to a patient for the treatment of disease.

Generally speaking, pharmaceutical formulation is the process in which different chemical substances are combined to a pure drug substance to produce a final drug product. Formulation studies involve developing a preparation of the drug which is both stable and acceptable to the patient. For orally taken drugs, this usually involves incorporating the drug into a tablet or a capsule. It is important to appreciate that a dosage form contains a variety of other substances apart from the drug itself, and studies have to be carried out to ensure that the drug is compatible with these other substances.

An excipient is an inactive substance used as a carrier for the active ingredients of a drug product, in this case a liposome comprising a TLR prodrug. In addition, excipients can be used to aid the process by which a drug product is manufactured. The active substance is then dissolved or mixed with an excipient. Excipients are also sometimes used to bulk up formulations with very potent active ingredients, to allow for convenient and accurate dosage. Once the active ingredient has been purified, it cannot stay in purified form for an extended amount of time. In many cases it will denature, fall out of solution, or stick to the sides of the container.

To stabilize the active ingredient, excipients are added to ensure that the active ingredient stays active and is stable for a long enough period of time that the shelf-life of the product makes it competitive with other products and safe for the end-user. Examples of excipients include but are not limited to, anti-adherents, binders, coatings, disintegrants, fillers, diluents, flavors, colors, lubricants, and preservatives. The final formulation comprises and active ingredient and excipients which are then enclosed in the pharmaceutical dosage form.

Pre-formulation involves the characterization of a drug's physical, chemical, and mechanical properties in order to choose what other ingredients should be used in the preparation. Formulation studies then consider such factors as stability, particle size, polymorphism, pH, and solubility, as all of these can influence bioavailability and hence the activity of a drug. The drug must be combined with inactive additives by a method which ensures that the quantity of drug present is consistent in each dosage unit (e.g., each vial). The dosage should have a uniform appearance.

It is unlikely that these studies will be complete by the time clinical trials commence. This means that simple preparations are developed initially for use in phase I clinical trials. These typically consist of vials, hand-filled capsules containing a small amount of the drug and a diluent. Proof of the long-term stability of these formulations is not required, as they will be used (tested) in a matter of days. However, long-term stability is critical in supply chain management since the time the final formulation is packaged until it reaches the patient can be several months or years. Consideration has to be given to what is called the drug load (i.e., the ratio of the active drug to the total contents of the dose). A low drug load may cause homogeneity problems. A high drug load may pose flow problems or require large capsules if the compound has a low bulk density. By the time phase III clinical trials are reached, the formulation of the drug should have been developed to be close to the preparation that will ultimately be used in the market.

A knowledge of stability is essential by this stage, and conditions must have been developed to ensure that the drug is stable in the preparation. If the drug proves unstable, it will invalidate the results from clinical trials since it would be impossible to know what the administered dose actually was. Stability studies are carried out to test whether temperature, humidity, oxidation, or photolysis (ultraviolet light or visible light) have any effect, and the preparation is analyzed to see if any degradation products have been formed. It is also important to check whether there are any unwanted interactions between the preparation and the container. If a plastic container is used, tests are carried out to see whether any of the ingredients become adsorbed on to the plastic, and whether any plasticizers, lubricants, pigments, or stabilizers leach out of the plastic into the preparation. Even the adhesives for the container label need to be tested, to ensure they do not leach through the plastic container into the preparation. The way a drug is formulated can avoid some of the problems associated with oral administration. Drugs are normally taken orally as tablets or capsules. The drug (active substance) itself needs to be soluble in aqueous solution at a controlled rate. Such factors as particle size and crystal form can significantly affect dissolution. Fast dissolution is not always ideal. For example, slow dissolution rates can prolong the duration of action or avoid initial high plasma levels.

In some embodiments, the nanocarrier (e.g., SLNP or a liposome comprising a TLR prodrug) and/or the liposome comprising a TLR prodrug and co-formulated with an immune modulating agent are administered alone or in a mixture with a physiologically acceptable carrier (such as physiological saline or phosphate buffer) selected in accordance with the route of administration and standard pharmaceutical practice. For example, when used as an injectable, the nanocarriers can be formulated as a sterile suspension, dispersion, or emulsion with a pharmaceutically acceptable carrier. In certain embodiments normal saline can be employed as the pharmaceutically acceptable carrier. Other suitable carriers include, e.g., water, buffered water, 0.4% saline, 0.3% glycine, 5% glucose and the like, including glycoproteins for enhanced stability, such as albumin, lipoprotein, globulin, etc. In compositions comprising saline or other salt-containing carriers, the carrier is preferably added following nanocarrier formation. Thus, after the nanocarrier is formed and loaded with suitable drug(s), the nanocarrier can be diluted into pharmaceutically acceptable carriers such as normal saline. Similarly, the TLR prodrug liposomes can be introduced into carriers that facilitate suspension of the nanomaterials (e.g., emulsions, dilutions, etc.).

The pharmaceutical compositions may be sterilized by conventional, well-known sterilization techniques. The resulting aqueous solutions, suspensions, dispersions, emulsions, etc., may be packaged for use or filtered under aseptic conditions. In certain embodiments the drug delivery nanocarriers (e.g., LNP or SLNP-coated nanoparticles) are lyophilized, the lyophilized preparation being combined with a sterile aqueous solution prior to administration. The compositions may also contain pharmaceutically acceptable auxiliary substances as required to approximate physiological conditions, such as pH-adjusting and buffering agents, tonicity adjusting agents and the like, for example, sodium acetate, sodium lactate, sodium chloride, potassium chloride, calcium chloride, etc.

Additionally, in certain embodiments, the pharmaceutical formulation may include lipid-protective agents that protect lipids against free-radical and lipid-peroxidative damage on storage. Lipophilic free-radical quenchers, such as alpha-tocopherol and water-soluble iron-specific chelators, such as ferrioxamine, are suitable and contemplated herein. The concentration of nanocarrier (e.g., SLNP or liposome comprising TLR prodrugs) in the pharmaceutical formulations can vary widely, e.g., from less than approximately 0.05%, usually at least approximately 2 to 5% to as much as 10 to 50%, or to 40%, or to 30% by weight and are selected primarily by fluid volumes, viscosities, etc., in accordance with the particular mode of administration selected. For example, the concentration may be increased to lower the fluid load associated with treatment. This may be particularly desirable in patients having atherosclerosis-associated congestive heart failure or severe hypertension. Alternatively, nanocarriers composed of irritating lipids may be diluted to low concentrations to lessen inflammation at the site of administration. The amount of nanocarriers administered will depend upon the particular drug used, the disease state being treated and the judgment of the clinician but will generally be between approximately 0.01 and approximately 50 mg per kilogram of body weight, preferably between approximately 0.1 and approximately 5 mg per kg of body weight.

One of skill in the art will appreciate that exact dosages will vary depending upon such factors as the particular TLR prodrugs and any co-formulated immune modulating agents and the desirable medical effect, as well as patient factors such as age, sex, general condition, and the like. Those of skill in the art can readily take these factors into account and use them to establish effective therapeutic concentrations without resort to undue experimentation.

For administration to humans (or to non-human mammals) in the curative, remissive, retardive, or prophylactic treatment of diseases described herein the prescribing physician will ultimately determine the appropriate dosage of the drug for a given human (or non-human) subject, and this can be expected to vary according to the age, weight, and response of the individual as well as the nature and severity of the patient's disease. In certain embodiments the dosage of the drug provided by the nanocarrier(s) can be approximately equal to that employed for the free drug. However as noted above, the nanocarriers described herein can significantly reduce the toxicity of the drug(s) administered thereby and significantly increase a therapeutic window. Accordingly, in some cases dosages in excess of those prescribed for the free drug(s) will be utilized.

One of ordinary skill in the art will appreciate and be enabled to make variations and modifications to the disclosed embodiment without altering the function and purpose of the invention disclosed herein. Such variations and modifications are intended within the scope of the present disclosure.

IX.) Combination Therapy

As the skilled artisan will appreciate and understand, cancer cell growth and survival can be impacted by multiple signaling pathways. Thus, it is useful to combine different enzyme/protein/receptor inhibitors, exhibiting different preferences in the targets which they modulate the activities of, to treat such conditions. Targeting more than one signaling pathway (or more than one biological molecule involved in a given signaling pathway) may reduce the likelihood of drug-resistance arising in a cell population, and/or reduce the toxicity of treatment.

Thus, the liposomes or SLNPs comprising TLR prodrugs of the present disclosure can be used in combination with one or more other enzyme/protein/receptor inhibitors or one or more therapies for the treatment of diseases, such as cancer or infections. Examples of diseases and indications treatable with combination therapies include those set forth in the present disclosure. Examples of cancers include, but are not limited to, solid tumors and liquid tumors, such as blood cancers. Examples of infections include viral infections, bacterial infections, fungus infections or parasite infections.

For example, the liposomes or SLNPs comprising TLR prodrugs of the present disclosure can be combined with one or more inhibitors of the following kinases for the treatment of cancer: Akt1, Akt2, Akt3, TGF-βR, PKA, PKG, PKC, CaM-kinase, phosphorylase kinase, MEKK, ERK, MAPK, mTOR, EGFR, HER2, HER3, HER4, INS-R, IGF-1R, IR-R, PDGFαR, PDGFβR, PI3K (alpha, beta, gamma, delta), CSFIR, KIT, FLK-II, KDR/FLK-1, FLK-4, flt-1, FGFR1, FGFR2, FGFR3, FGFR4, c-Met, Ron, Sea, TRKA, TRKB, TRKC, TAM kinases (Axl, Mer, Tyro3), FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf.

In further embodiments, the liposomes or SLNPs comprising TLR prodrugs of the present disclosure can be combined with one or more of the following inhibitors for the treatment of cancer or infections. Non-limiting examples of inhibitors that can be combined with the compounds of the present disclosure for treatment of cancer and infections include an FGFR inhibitor (FGFR1, FGFR2, FGFR3 or FGFR4, e.g., INCB54828, INCB62079 and INCB63904), a JAK inhibitor (JAK1 and/or JAK2, e.g., ruxolitinib, baricitinib or INCB39110), a TLR inhibitor (e.g., epacadostat, NLG919, or BMS-986205), an LSD1 inhibitor (e.g., INCB59872 and INCB60003), a TDO inhibitor, a PI3K-delta inhibitor (e.g., INCB50797 and INCB50465), a PI3K-gamma inhibitor such as PI3K-gamma selective inhibitor, a Pim inhibitor (e.g., INCB53914), a CSF1R inhibitor, a TAM receptor tyrosine kinases (Tyro-3, Axl, and Mer), an adenosine receptor antagonist (e.g., A2a/A2b receptor antagonist), an HPK1 inhibitor, a histone deacetylase inhibitor (HDAC) such as an HDAC8 inhibitor, an angiogenesis inhibitor, an interleukin receptor inhibitor, bromo and extra terminal family members inhibitors (for example, bromodomain inhibitors or BET inhibitors such as INCB54329 and INCB57643), a poly ADP ribose polymerase (PARP) inhibitor such as rucaparib, olaparib, niraparib, veliparib, or talazoparib, an arginase inhibitor (INCB01158), a PD-1 inhibitor, a PD-1/L-1 inhibitor, a PD-1/L-2 inhibitor, and an adenosine receptor antagonist or combinations thereof.

In further embodiments, the liposomes or SLNPs comprising TLR prodrugs of the present disclosure can be combined with one or more activator of invariant natural killer T (iNKT) cells including but not limited to, α-galactosylceramida (α-GalCer) and analogs thereof including, C8 Galactosyl(α) Ceramide, C16 Galactosyl(α) Ceramide, and C24:1 Galactosyl(α) Ceramide (Avanti Polar Lipids, Alabaster, Alabama).

Additionally, the liposomes or SLNPs comprising TLR prodrugs of the present disclosure can further be used in combination with other methods of treating cancers, for example by chemotherapy, irradiation therapy, tumor-targeted therapy, adjuvant therapy, immunotherapy, or surgery.

Examples of immunotherapy include cytokine treatment (e.g., interferons, GM-CSF, G-CSF, IL-2), CRS-207 immunotherapy, cancer vaccine, monoclonal antibody, adoptive T cell transfer, Toll receptor agonists, STING agonists, oncolytic virotherapy and immunomodulating small molecules, including thalidomide or JAK1/2 inhibitor and the like.

The liposomes or SLNPs comprising TLR prodrugs can be administered in combination with one or more anti-cancer drugs, such as a chemotherapeutics. Example chemotherapeutics include any of: abarelix, aldesleukin, alemtuzumab, alitretinoin, allopurinol, altretamine, anastrozole, arsenic trioxide, asparaginase, azacitidine, bevacizumab, bexarotene, baricitinib, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalTLRmide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, olaparib, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, rucaparib, sorafenib, streptozocin, sunitinib, sunitinib maleate, tamoxifen, temozolomide, teniposide, testolactone, thalTLRmide, thioguanine, thiotepa, topotecan, toremifene, tositumomab, trastuzumab, tretinoin, uracil mustard, valrubicin, vinblastine, vincristine, vinorelbine, vorinostat, niraparib, veliparib, talazoparib and zoledronate.

Other anti-cancer agent(s) include antibody therapeutics such as trastuzumab (Herceptin), antibodies to costimulatory molecules such as CTLA-4 (e.g., ipilimumab), 4-1BB (e.g., urelumab, utomilumab), antibodies to PD-1 and PD-L1/L2, or antibodies to cytokines (IL-10, TGF-.beta., etc.).

Examples of antibodies to PD-1 and/or PD-L1/L2 that can be combined with compounds of the present disclosure for the treatment of cancer or infections such as viral, bacteria, fungus and parasite infections include, but are not limited to, nivolumab, pembrolizumab, MPDL3280A, MEDI-4736 and SHR-1210.

In addition, liposomes or SLNPs comprising TLR prodrugs of the present disclosure can be used in combination with one or more immune checkpoint inhibitors for the treatment of diseases, such as cancer or infections. Exemplary immune checkpoint inhibitors include inhibitors against immune checkpoint molecules such as CD27, CD28, CD40, CD122, CD96, CD73, CD47, OX40, GITR, CSF1R, JAK, PI3K delta, PI3K gamma, TAM, arginase, CD137 (also known as 4-1BB), ICOS, A2AR, B7-H3, B7-H4, BTLA, CTLA-4, LAG3, TIM3, VISTA, PD-1, PD-L1 and PD-L2.

In some embodiments, the immune checkpoint molecule is a stimulatory checkpoint molecule selected from CD27, CD28, CD40, ICOS, OX40, GITR and CD137. In further embodiments, the immune checkpoint molecule is an inhibitory checkpoint molecule selected from A2AR, B7-H3, B7-H4, BTLA, CTLA-4, TLR, KIR, LAG3, PD-1, TIM3, and VISTA. In further embodiments, the liposomes comprising TLR prodrugs provided herein can be used in combination with one or more agents selected from KIR inhibitors, TIGIT inhibitors, LAIR1 inhibitors, CD160 inhibitors, 2B4 inhibitors and TGF beta (“TGFb”) inhibitors.

X.) Methods of Delivering Liposomes Comprising TLR Prodrugs to a Cell Expressing Toll-Like Receptor (“TLR”)

As it is known in the art, a wide variety of compositions and methods for using prodrugs and/or nanocarriers to kill tumor cells are known in the art. In the context of cancers, typical methods entail administering to a mammal having a tumor, a biologically effective amount of a TLR prodrug of the disclosure, and/or a nanocarrier of the disclosure comprising a TLR prodrug.

A typical embodiment is a method of delivering a therapeutic agent to a cell expressing TLR1/2, TLR4, TLR7, TLR8, and/or TLR7/8, comprising forming a TLR prodrug by conjugating a drug moiety of the disclosure with a lipid of the disclosure via a Linkage Unit, and exposing the cell to the TLR prodrug.

In one embodiment, the TLR prodrug comprises a drug moiety of Formula I and CHEMS conjugated via a LU comprising a hydromethylcarbamate linker.

In one embodiment, the TLR prodrug comprises a drug moiety of Formula I and Stearic Acid conjugated via a LU comprising a hydromethylcarbamate linker.

In one embodiment, the TLR prodrug comprises a drug moiety of Formula I and non-cleavable Stearic Acid derivative.

In one embodiment, the TLR prodrug comprises TR12 and CHEMS conjugated via a LU comprising a hydromethylcarbamate linker.

In one embodiment, the TLR prodrug comprises TR12 and Stearic Acid conjugated via a LU comprising a hydromethylcarbamate linker.

Another illustrative embodiment is a method of treating an individual suspected of suffering from metastasized cancer, comprising a step of administering parenterally to said individual a pharmaceutical composition comprising a therapeutically effective amount of a TLR prodrug produced by conjugating a drug moiety with a lipid of the disclosure via a Linkage Unit, and exposing the cell to the TLR prodrug.

In one embodiment, the TLR prodrug comprises a drug moiety of Formula I and CHEMS conjugated via a LU comprising a hydromethylcarbamate linker.

In one embodiment, the TLR prodrug comprises a drug moiety of Formula I and Stearic Acid conjugated via a LU comprising a hydromethylcarbamate linker.

In one embodiment, the TLR prodrug comprises a drug moiety of Formula I and non-cleavable Stearic Acid derivative.

In one embodiment, the TLR prodrug comprises TR12 and CHEMS conjugated via a LU comprising a hydromethylcarbamate linker.

In one embodiment, the TLR prodrug comprises TR12 and Stearic Acid conjugated via a LU comprising a hydromethylcarbamate linker.

TLR prodrugs, nanocarriers, liposomes, co-formulated nanocarriers and co-formulated liposomes of the present disclosure inhibit the activity of TLR protein/protein interaction and, thus, are useful in treating diseases and disorders associated with activity of TLR and the diseases and disorders.

In further embodiments of the disclosure, the TLR prodrugs, nanocarriers, or pharmaceutically acceptable salts or stereoisomers thereof, are useful for therapeutic administration to enhance, stimulate and/or increase immunity in cancer, chronic infection, or sepsis, including enhancement of response to vaccination.

In further embodiments, the present disclosure provides a method for inhibiting the TLR (e.g., TLR1/2, TLR4, TLR7, TLR8, and/or TLR7/8) T-cell function. The method includes administering to an individual or a patient a TLR prodrug, liposomes, nanocarriers, and/or of any of the formulas as described herein (e.g., TR12 and/or TR13), or of a TLR prodrug, nanocarrier, and nano-encapsulated TLR inhibitor prodrugs as recited in any of the claims and described herein, or a pharmaceutically acceptable salt or a stereoisomer thereof. The TLR prodrug, liposomes, nanocarriers, and nano-encapsulated TLR inhibitor prodrugs of the present disclosure can be used alone, in combination with other agents or therapies or as an adjuvant or neoadjuvant for the treatment of diseases or disorders, including cancer and other diseases. For the uses and methods described herein, any of the TLR prodrugs, liposomes, nanocarriers, and nano-encapsulated TLR prodrugs of the disclosure, including any of the embodiments thereof, may be used.

In addition, The TLR prodrugs, liposomes, nanocarriers, and nano-encapsulated TLR inhibitor prodrugs of the present disclosure inhibit the TLR function, resulting in a TLR pathway blockade.

In further embodiments, the present disclosure provides treatment of an individual or a patient in vivo using TLR prodrugs, liposomes, nanocarriers, and nano-encapsulated TLR inhibitor prodrug or a salt or stereoisomer thereof such that growth of cancerous tumors is inhibited.

TLR prodrugs, liposomes, and nano-encapsulated TLR inhibitor prodrugs, or of any of the formulas as described herein (e.g., TR12 and/or TR13), or TLR prodrugs, liposomes, nanocarriers, and nano-encapsulated TLR inhibitor prodrugs as recited in any of the claims and described herein, or a salt or stereoisomer thereof, can be used to inhibit the growth of cancerous tumors.

In the alternative, TLR prodrugs, liposomes, nanocarriers, and nano-encapsulated TLR prodrugs of the disclosure, or of any of the formulas as described herein, or a compound as recited in any of the claims and described herein (e.g., TR12 and/or TR13), or a salt or stereoisomer thereof, can be used in conjunction with other agents or standard cancer treatments, as described in this disclosure.

In a further embodiment, the present disclosure provides a method for inhibiting growth of tumor cells in vitro. The method includes contacting the tumor cells in vitro with TLR prodrugs, liposomes, nanocarriers, and nano-encapsulated TLR inhibitor prodrugs of the disclosure, or of any of the formulas as described herein (e.g., TR12 and/or TR13), or of a TLR prodrug, liposomes, nanocarriers, and nano-encapsulated TLR inhibitor prodrugs as recited in any of the claims and described herein, or of a salt or stereoisomer thereof.

In a further embodiment, the present disclosure provides a method for inhibiting growth of tumor cells in a patient. The method includes contacting the tumor cells with TLR prodrugs, liposomes, nanocarriers, and nano-encapsulated TLR inhibitor prodrugs of the disclosure, or of any of the formulas as described herein (e.g., TR12 and/or TR13), or of a TLR prodrug, liposomes, and nano-encapsulated TLR inhibitor prodrugs as recited in any of the claims and described herein, or of a salt or stereoisomer thereof.

XI.) Methods of Treating Cancer(s) and Other Immunological Disorder(s)

Another embodiment of the present disclosure is a method for treating cancer. The method comprises administering to a patient, a therapeutically effective amount of a liposome comprising a TLR prodrug (i.e., TR12 and/or TR13) herein, a compound as recited in any of the claims and described herein, or a salt thereof. Examples of cancers include those whose growth may be inhibited using TLR inhibitors of the disclosure and TLR prodrugs of the disclosure and cancers typically responsive to immunotherapy.

In some embodiments, the present disclosure provides a method of enhancing, stimulating and/or increasing the immune response in a patient. The method includes administering to the patient a therapeutically effective amount of a TLR prodrug and/or a nanocarrier comprising the same (i.e., TR12 and/or TR13), a compound or composition as recited in any of the claims and described herein, or a salt thereof.

Non-limiting examples of cancers that are treatable using the nanocarriers comprising TLR prodrugs, TLR prodrugs and co-formulated nanocarriers of the present disclosure include, but are not limited to, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. The compounds of the present disclosure are also useful for the treatment of metastatic cancers, especially metastatic cancers that express TLR.

In some embodiments, cancers treatable with nanocarriers, or TLR prodrugs of the present disclosure include melanoma (e.g., metastatic malignant melanoma), renal cancer (e.g., clear cell carcinoma), prostate cancer (e.g., hormone refractory prostate adenocarcinoma), breast cancer, colon cancer, lung cancer (e.g. non-small cell lung cancer and small cell lung cancer), squamous cell head and neck cancer, urothelial cancer (e.g. bladder) and cancers with high microsatellite instability (MSI^{high}). Additionally, the disclosure includes refractory or recurrent malignancies whose growth may be inhibited using the liposomes, or TLR prodrugs or co-formulated liposomes of the disclosure.

In additional embodiments, cancers that are treatable using the formulated and/or co-formulated nanocarriers or TLR prodrugs of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer, etc.), hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), DLBCL, mantle cell lymphoma, Non-Hodgkin lymphoma (including relapsed or refractory NHL and recurrent follicular), Hodgkin lymphoma or multiple myeloma) and combinations of said cancers.

In further embodiments, cancers that are treatable using the formulated and/or co-formulated nanocarriers or TLR prodrugs of the present disclosure include, but are not limited to, cholangiocarcinoma, bile duct cancer, triple negative breast cancer, rhabdomyosarcoma, small cell lung cancer, leiomyosarcoma, hepatocellular carcinoma, Ewing's sarcoma, brain cancer, brain tumor, astrocytoma, neuroblastoma, neurofibroma, basal cell carcinoma, chondrosarcoma, epithelioid sarcoma, eye cancer, Fallopian tube cancer, gastrointestinal cancer, gastrointestinal stromal tumors, hairy cell leukemia, intestinal cancer, islet cell cancer, oral cancer, mouth cancer, throat cancer, laryngeal cancer, lip cancer, mesothelioma, neck cancer, nasal cavity cancer, ocular cancer, ocular melanoma, pelvic cancer, rectal cancer, renal cell carcinoma, salivary gland cancer, sinus cancer, spinal cancer, tongue cancer, tubular carcinoma, urethral cancer, and ureteral cancer.

In addition, in some embodiments, the formulated and/or co-formulated nanocarriers, or TLR prodrugs of the present disclosure can be used to treat sickle cell disease and sickle cell anemia.

Furthermore, in some embodiments, diseases and indications that are treatable using the formulated and/or co-formulated nanocarriers, or TLR prodrugs of the present disclosure include, but are not limited to hematological cancers, sarcomas, lung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), and essential thrombocytosis (ET)), myelodysplasia syndrome (MDS), T-cell acute lymphoblastic lymphoma (T-ALL) and multiple myeloma (MM).

Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, and teratoma.

Exemplary lung cancers include non-small cell lung cancer (NSCLC), small cell lung cancer, bronchogenic carcinoma (squamous cell, undifferentiated small cell, undifferentiated large cell, adenocarcinoma), alveolar (bronchiolar) carcinoma, bronchial adenoma, chondromatous hamartoma, and mesothelioma.

Exemplary gastrointestinal cancers include cancers of the esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), and colorectal cancer.

Exemplary genitourinary tract cancers include cancers of the kidney (adenocarcinoma, Wilm's tumor [nephroblastoma]), bladder and urethra (squamous cell carcinoma, transitional cell carcinoma, adenocarcinoma), prostate (adenocarcinoma, sarcoma), and testis (seminoma, teratoma, embryonal carcinoma, teratocarcinoma, choriocarcinoma, sarcoma, interstitial cell carcinoma, fibroma, fibroadenoma, adenomatoid tumors, lipoma).

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors.

Exemplary nervous system cancers include cancers of the skull (osteoma, hemangioma, granuloma, xanthoma, osteitis deformans), meninges (meningioma, meningiosarcoma, gliomatosis), brain (astrocytoma, meduoblastoma, glioma, ependymoma, germinoma (pinealoma), glioblastoma, glioblastoma multiform, oligodendroglioma, schwannoma, retinoblastoma, congenital tumors), and spinal cord (neurofibroma, meningioma, glioma, sarcoma), as well as neuroblastoma and Lhermitte-Duclos disease.

Exemplary gynecological cancers include cancers of the uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, squamous cell carcinoma, Kaposi's sarcoma, moles dysplastic nevi, lipoma, angioma, dermatofibroma, and keloids. In some embodiments, diseases and indications that are treatable using the compounds of the present disclosure include, but are not limited to, sickle cell disease (e.g., sickle cell anemia), triple-negative breast cancer (TNBC), myelodysplastic syndromes, testicular cancer, bile duct cancer, esophageal cancer, and urothelial carcinoma.

Additionally, TLR and/or kynurenine pathway blockade with formulated and/or co-formulated nanocarriers, or TLR prodrugs of the present disclosure can also be used for treating infections such as viral, bacteria, fungus, and parasite infections.

The present disclosure provides a method for treating infections such as viral infections. The method includes administering to a patient, a therapeutically effective amount of a formulated and/or co-formulated nanocarrier or TLR prodrugs or any of the formulas as described herein (i.e., TR12 and/or TR13) as recited in any of the claims and described herein, a salt thereof.

Examples of viruses causing infections treatable by methods of the present disclosure include, but are not limit to, human immunodeficiency virus, human papillomavirus, influenza, hepatitis A, B, C or D viruses, adenovirus, poxvirus, herpes simplex viruses, human cytomegalovirus, severe acute respiratory syndrome virus, Ebola virus, and measles virus. In some embodiments, viruses causing infections treatable by methods of the present disclosure include, but are not limit to, hepatitis (A, B, or C), herpes virus (e.g., VZV, HSV-1, HAV-6, HSV-II, and CMV, Epstein Barr virus), adenovirus, influenza virus, flaviviruses, echovirus, rhinovirus, coxsackie virus, coronavirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

In addition, the present disclosure provides a method for treating bacterial infections. The method includes administering to a patient, a therapeutically effective amount of a formulated and/or co-formulated nanocarriers or TLR prodrugs, or any of the formulas as described herein (i.e., TR12 and/or TR13) as recited in any of the claims and described herein, or a salt thereof.

Examples of pathogenic bacteria causing infections treatable by methods of the disclosure, include but are not limited to, chlamydia, rickettsia bacteria, mycobacteria, staphylococci, streptococci, pneumonococci, meningococci and conococci, klebsiella, proteus, serratia, pseudomonas, legionella, diphtheria, salmonella, bacilli, cholera, tetanus, botulism, anthrax, plague, leptospirosis, and Lyme's disease bacteria.

In addition, the present disclosure provides a method for treating fungus infections. The method includes administering to a patient, a therapeutically effective amount of a formulated and/or co-formulated nanocarriers or TLR prodrugs, or any of the formulas as described herein (i.e., TR12 and/or TR13) as recited in any of the claims and described herein, or a salt thereof.

Examples of pathogenic fungi causing infections treatable by methods of the disclosure include, but are not limited to, Candida (albicans, krusei, glabrata, tropicalis, etc.), Cryptococcus neoformans, Aspergillus (fumigatus, Niger, etc.), Genus Mucorales (Mucor, absidia, rhizophus), Sporothrix schenkii, Blastomyces dermatitidis, Paracoccidioides brasiliensis, Coccidioides immitis and Histoplasma capsulatum.

Additionally, the present disclosure provides a method for treating parasite infections. The method includes administering to a patient, a therapeutically effective amount of a formulated and/or co-formulated nanocarriers or TLR prodrugs, or any of the formulas as described herein (i.e., TR12 and/or TR13) as recited in any of the claims and described herein, or a salt thereof.

Examples of pathogenic parasites causing infections treatable by methods of the disclosure include, but are not limited to, Entamoeba histolytica, Balantidium coli, Naegleriafowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and Nippostrongylus brasiliensis.

In a further set of embodiments that are within the scope of this disclosure, the formulated and/or co-formulated nanocarriers, or TLR prodrugs, or any of the formulas as described herein (i.e., TR12 and/or TR13) are useful in preventing or reducing the risk of developing any of the diseases referred to in this disclosure; e.g., preventing or reducing the risk of developing a disease, condition or disorder in an individual who may be predisposed to the disease, condition or disorder but does not yet experience or display the pathology or symptomatology of the disease.

In one embodiment, the methods described herein comprise LNP-TR12 and/or a therapeutically effective amount of LNP-TR12.

In one embodiment, the methods described herein comprise SLNP-TR12 and/or a therapeutically effective amount of SLNP-TR12.

In one embodiment, the methods described herein comprise LNP-TR13 and/or a therapeutically effective amount of LNP-TR13.

In one embodiment, the methods described herein comprise SLNP-TR13 and/or a therapeutically effective amount of SLNP-TR13.

In one embodiment, the methods described herein comprise SLNP-TR12-IC1 and/or a therapeutically effective amount of SLNP-TR12-IC1.

In one embodiment, the methods described herein comprise SLNP-TR12-IC1 wherein the ratio is set forth as 8:1.

In one embodiment, the methods described herein comprise SLNP-TR12-IC1 wherein the ratio is set forth as 16:1.

In one embodiment, the methods described herein comprise SLNP-TR12-AR5 and/or a therapeutically effective amount of SLNP-TR12-AR5.

In one embodiment, the methods described herein comprise SLNP-TR12-NTI-47C and/or a therapeutically effective amount of SLNP-TR12-NTI-47C.

XII.) Kits/Articles of Manufacture

For use in the laboratory, prognostic, prophylactic, diagnostic, and therapeutic applications described herein, kits are within the scope of the invention. Such kits can comprise a carrier, package, or container that is compartmentalized to receive one or more containers such as vials, tubes, and the like, each of the container(s) comprising one of the separate elements to be used in the method, along with a label or insert comprising instructions for use, such as a use described herein. For example, the container(s) can comprise a formulated and/or co-formulated nanocarriers that is or can be detectably labeled and/or is loaded with a TLR prodrug of the disclosure. Kits can comprise a container comprising a drug unit. The kit can include all or part of the formulated and/or co-formulated nanocarrier, liposomes, SLNPs, and/or a TLR prodrug.

The kit of the invention will typically comprise the container described above, and one or more other containers associated therewith that comprise materials desirable from a commercial and user standpoint, including buffers, diluents, filters, needles, syringes; carrier, package, container, vial and/or tube labels listing contents and/or instructions for use, and package inserts with instructions for use.

A label can be present on or with the container to indicate that the composition is used for a specific therapy or non-therapeutic application, such as a prognostic, prophylactic, diagnostic, or laboratory application, and can also indicate directions for either in vivo or in vitro use, such as those described herein. Directions and or other information can also be included on an insert(s) or label(s) which is included with or on the kit. The label can be on or associated with the container. A label can be on a container when letters, numbers or other characters forming the label are molded or etched into the container itself; a label can be associated with a container when it is present within a receptacle or carrier that also holds the container, e.g., as a package insert. The label can indicate that the composition is used for diagnosing, treating, prophylaxing or prognosing a condition, such as a cancer or other immunological disorder.

The terms “kit” and “article of manufacture” can be used as synonyms.

In another embodiment of the invention, an article(s) of manufacture containing compositions, such as formulated and/or co-formulated nanocarrier and/or TLR prodrugs are within the scope of this disclosure. The article of manufacture typically comprises at least one container and at least one label. Suitable containers include, for example, bottles, vials, syringes, and test tubes. The containers can be formed from a variety of materials such as glass, metal, or plastic. The container can hold formulated and/or co-formulated nanocarrier loaded with TLR prodrugs.

The container can alternatively hold a composition that is effective for treating, diagnosis, prognosing or prophylaxing a condition and can have a sterile access port (for example the container can be an intravenous solution bag or a vial having a stopper pierceable by a hypodermic injection needle). The active agents in the composition can be formulated and/or co-formulated nanocarrier loaded with TLR prodrugs and/or TLR prodrugs as disclosed herein.

The article of manufacture can further comprise a second container comprising a pharmaceutically acceptable buffer, such as phosphate-buffered saline, Ringer's solution and/or dextrose solution. It can further include other materials desirable from a commercial and user standpoint, including other buffers, diluents, filters, stirrers, needles, syringes, and/or package inserts with indications and/or instructions for use.

In one embodiment, the kits described herein comprise LNP-TR12 and/or a therapeutically effective amount of LNP-TR12.

In one embodiment, the kits described herein comprise SLNP-TR12 and/or a therapeutically effective amount of SLNP-TR12.

In one embodiment, the kits described herein comprise LNP-TR13 and/or a therapeutically effective amount of LNP-TR13.

In one embodiment, the kits described herein comprise SLNP-TR13 and/or a therapeutically effective amount of SLNP-TR13.

In one embodiment, the kits described herein comprise SLNP-TR12-IC1 and/or a therapeutically effective amount of SLNP-TR12-IC1.

In one embodiment, the methods described herein comprise SLNP-TR12-IC1 wherein the ratio is set forth as 8:1.

In one embodiment, the methods described herein comprise SLNP-TR12-IC1 wherein the ratio is set forth as 16:1.

In one embodiment, the kits described herein comprise SLNP-TR12-AR5 and/or a therapeutically effective amount of SLNP-TR12-AR5.

In one embodiment, the kits described herein comprise SLNP-TR12-NTI-47C and/or a therapeutically effective amount of SLNP-TR12-NTI-47C.

### EXEMPLARY EMBODIMENTS

1) A TLR prodrug composition comprising,
(i) a drug moiety;
(ii) a lipid moiety; and
(iii) a linkage unit (“LU”),
   whereby the drug moiety comprises a TLR agonist and whereby the LU conjugates the drug moiety with the lipid moiety.

2) The TLR prodrug of claim 1, further comprising the chemical structure set forth in FORMULA I.

3) The TLR prodrug of claim 1, wherein the drug moiety comprises the chemical structure set forth as TR12.

4) The TLR prodrug of claim 1, wherein the drug moiety comprises the chemical structure set forth as TR13.

5) The TLR prodrug of claim 1, wherein the LU is a hydromethylcarbamate linker.

6) The TLR prodrug of claim 1, wherein the lipid moiety comprises a lipid set forth in Table I.

7) The TLR prodrug of claim 1, wherein the lipid moiety comprises a lipid set forth in Table III.

8) The TLR prodrug of claim 1, wherein the lipid moiety comprises Stearic Acid.

9) The TLR prodrug of claim 1, wherein the lipid moiety comprises Stearic Acid and has the following chemical structure:

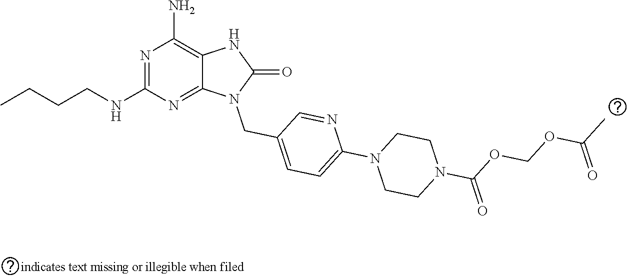 [SMILES: CCCCNc1nc(N)c2[nH]c(=O)n(Cc3ccc(N4CCN(C(=O)OCOC(C)=O)CC4)nc3)c2n1]

10) The TLR prodrug of claim 4, wherein the lipid moiety comprises a non-cleavable Stearic Acid derivative.

11) The TLR prodrug of claim 10, wherein the lipid moiety comprises a non-cleavable Stearic Acid derivative and has the following chemical structure:

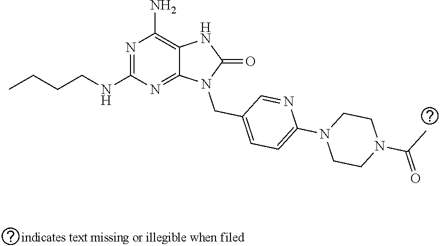 [SMILES: CCCCNc1nc(N)c2[nH]c(=O)n(Cc3ccc(N4CCN(C(C)=O)CC4)nc3)c2n1]

12) A TLR prodrug composition comprising,
(i) a drug moiety, whereby the drug moiety comprises TR12;
(ii) a lipid moiety, whereby the lipid moiety comprises Stearic Acid; and
(iii) LU, whereby the LU comprises a hydromethylcarbamate linker.

13) The TLR prodrug composition of claim 12, comprising the following chemical structure:

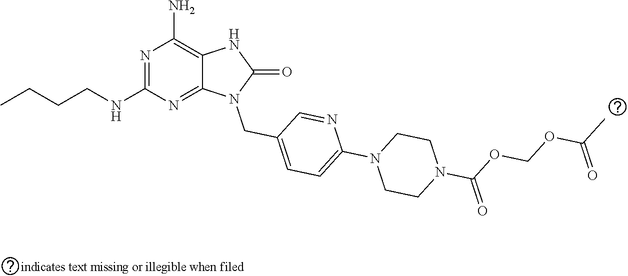 [SMILES: CCCCNc1nc(N)c2[nH]c(=O)n(Cc3ccc(N4CCN(C(=O)OCOC(C)=O)CC4)nc3)c2n1]

14) A TLR prodrug composition comprising,
(iv) a drug moiety, whereby the drug moiety comprises TR12;
(v) a lipid moiety, whereby the lipid moiety comprises a non-cleavable Stearic Acid derivative;

15) The TLR prodrug composition of claim 14, comprising the following chemical structure:

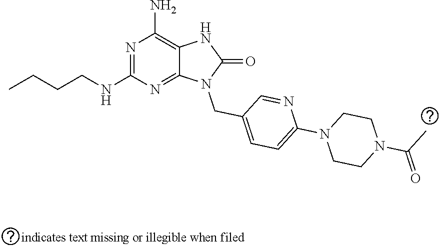 [SMILES: CCCCNc1nc(N)c2[nH]c(=O)n(Cc3ccc(N4CCN(C(C)=O)CC4)nc3)c2n1]

16) A liposome comprising, a TLR prodrug whereby the liposome releases an active TLR inhibitor after cleavage of a LU.

17) A nanocarrier comprising, an TLR prodrug whereby the nanocarrier releases an active TLR agonist after cleavage of a LU.

18) The nanocarrier of claim 17, wherein the LU is a hydromethylcarbamate linker.

19) The nanocarrier of claim 17, further comprising a helper lipid, whereby the helper lipid is set forth in Table II.

20) The nanocarrier of claim 17, wherein the TLR prodrug comprises TR12.

21) The nanocarrier of claim 17, wherein the nanocarrier is a liposome.

22) The liposome of claim 21, wherein the TLR prodrug comprises TR12 and is denoted LNP-TR12.

23) The liposome of claim 21, whereby the liposome is further co-formulated with one or more immune modulating agent or a lipid-prodrug thereof, wherein the immune modulating agent is selected from the group consisting of immunogenic-cell death inducing chemotherapeutics, A2aR inhibitors, STING agonists, CTLA-4 inhibitors, IDO inhibitors, PD-1/PD-L1 inhibitors, CD1D agonists and/or prodrugs thereof.

24) The liposome of claim 21, whereby the liposome is further co-formulated with an ICD-inducing chemotherapeutic, wherein the ICD-inducing chemotherapeutic is selected from the group consisting of DOX, MTO, OXA, CP, Bortezomib, Carfilzimib, IC1, or Paclitaxel.

25) The liposome of claim 21, further comprising DOX.

26) The liposome of claim 21, further comprising MTO.

27) The liposome of claim 24, further comprising DOX.

28) The liposome of claim 24, further comprising MTO.

29) The liposome of claim 21, whereby the liposome is further co-formulated with a toll-receptor agonist or a lipid-prodrug thereof, wherein the toll-receptor agonist is selected from the group consisting of Resiquimod (R848), Gardiquimod, 852A, DSR 6434, Telratolimod, CU-T12-9, monophosphoryl Lipid A (MPLA), 3D(6-acyl)-PHAD®, SMU127, Pam3CSK4, TR5, TR6, TR3, or 3D-PHAD®.

30) The liposome of claim 21, whereby the liposome is further co-formulated with a PD-1/PD-L1 antagonist or a lipid-prodrug thereof, wherein the PD-1/PD-L1 antagonist is selected from the group consisting of AUNP12, CA-170, PD3, or BMS-986189.

31) The liposome of claim 21, whereby the liposome is further co-formulated with a TGFb antagonist, wherein the TGFb antagonist is selected from the group consisting of TB4.

32) The liposome of claim 21, whereby the liposome is further co-formulated with an IDO inhibitor, wherein the IDO inhibitor is selected from the group consisting of ID3.

33) A kit comprising a nanocarrier of any one of claims 1-21.

34) A kit comprising a liposome of any one of claims 22-32.

35) The nanocarrier of claim 17, wherein the nanocarrier is a solid-lipid nanoparticle (SLNP).

36) The nanocarrier of claim 20, wherein the nanocarrier is a solid-lipid nanoparticle (SLNP).

37) The SLNP of claim 36, wherein the TLR prodrug comprises TR12 and is denoted SLNP-TR12.

38) The nanocarrier of claim 36, denoted SLNP-TR12.

39) The SLNP of claim 17, whereby the SLNP is further co-formulated with one or more immune modulating agent or a lipid-prodrug thereof, wherein the immune modulating agent is selected from the group consisting of immunogenic-cell death inducing chemotherapeutics, A2aR inhibitors, STING agonists, CTLA-4 inhibitors, IDO inhibitors, PD-1/PD-L1 inhibitors, CD1D agonists and/or prodrugs thereof.

40) The SLNP of claim 17, whereby the SLNP is further co-formulated with an ICD-inducing chemotherapeutic, wherein the ICD-inducing chemotherapeutic is selected from the group consisting of DOX, MTO, OXA, CP, Bortezomib, Carfilzimib, IC1, or Paclitaxel.

41) The SLNP of claim 37, further comprising DOX.

42) The SLNP of claim 37, further comprising MTO.

43) The SLNP of claim 40, further comprising DOX.

44) The SLNP of claim 40, further comprising MTO.

45) The SLNP of claim 37, whereby the liposome is further co-formulated with a toll-receptor agonist or a lipid-prodrug thereof, wherein the toll-receptor agonist is selected from the group consisting of Resiquimod (R848), Gardiquimod, 852A, DSR 6434, Telratolimod, CU-T12-9, monophosphoryl Lipid A (MPLA), 3D(6-acyl)-PHAD®, SMU127, Pam3CSK4, or 3D-PHAD®.

46) The SLNP of claim 37, whereby the liposome is further co-formulated with a PD-1/PD-L1 antagonist or a lipid-prodrug thereof, wherein the PD-1/PD-L1 antagonist is selected from the group consisting of AUNP12, CA-170, or BMS-986189.

47) The SLNP of claim 37, whereby the SLNP is further co-formulated with a PD-1/PD-L1 antagonist or a lipid-prodrug thereof, wherein the PD-1/PD-L1 antagonist is selected from the group consisting of AUNP12, CA-170, PD3, or BMS-986189.

48) The SLNP of claim 37, whereby the SLNP is further co-formulated with a TGFb antagonist, wherein the TGFb antagonist is selected from the group consisting of TB4.

49) The SLNP of claim 37, whereby the SLNP is further co-formulated with an IDO inhibitor, wherein the IDO inhibitor is selected from the group consisting of ID3.

50) A kit comprising a SLNP of any one of claims 35-49.

51) A method of treating a subject suffering or diagnosed with cancer comprising,
(i) administering to a subject in need of such treatment an effective amount of a nanocarrier, wherein the nanocarrier comprises an TLR prodrug; and
(ii) a pharmaceutically acceptable salt thereof.

52) The method of claim 51, wherein the TLR prodrug comprises an TR12-Prodrug.

53) The method of claim 51, wherein the nanocarrier comprises an TR12-Prodrug further co-formulated with and ICD-inducing chemotherapeutic.

54) The method of claim 51, wherein the nanocarrier comprises an TR12-Prodrug further co-formulated with an immune modulating agent.

55) The method of claim 51, wherein the nanocarrier is a liposome.

56) The method of claim 51, wherein the liposome is LNP-TR12.

57) The method of claim 51, wherein the nanocarrier is a solid-lipid nanoparticle.

58) The method of claim 51, wherein the SLNP is SLNP-TR12.

59) The method of claim 56, wherein LNP-TR12 is used in combination with a PD-1 antibody, a CTLA4 antibody, or an immunogenic cell death inducing chemotherapy drug (e.g., DOX or MTO).

60) The method of claim 58, wherein SLNP-TR12 is used in combination with a PD-1 antibody, a CTLA4 antibody, or an immunogenic cell death inducing chemotherapy drug (e.g., DOX or MTO).

61) A method of treating a subject suffering or diagnosed with cancer comprising,
(iii) administering to a subject in need of such treatment an effective amount of a nanocarrier, wherein the nanocarrier comprises an TLR prodrug; and
(iv) a pharmaceutically acceptable salt thereof.

62) The method of claim 61, wherein the TLR prodrug comprises an TR12-Prodrug.

63) The method of claim 61, wherein the nanocarrier comprises an TR12-Prodrug further co-formulated with and ICD-inducing chemotherapeutic.

64) The method of claim 61, wherein the nanocarrier comprises an TR12-Prodrug further co-formulated with an immune modulating agent.

65) The method of claim 61, wherein the nanocarrier is a solid-lipid nanoparticle (“SLNP”).

66) The method of claim 61, wherein the SLNP is SLNP-TR12.

67) The method of claim 61, wherein the nanocarrier is a liposome.

68) The method of claim 61, wherein the liposome is LNP-TR12.

69) A TR12 Prodrug having the following chemical structure:

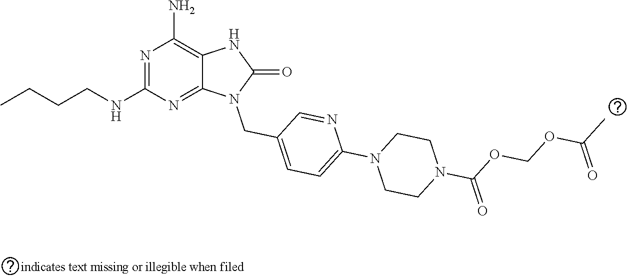 [SMILES: CCCCNc1nc(N)c2[nH]c(=O)n(Cc3ccc(N4CCN(C(=O)OCOC(C)=O)CC4)nc3)c2n1]

70) A liposome comprising the TR12 Prodrug of claim 69.

71) A liposome comprising the TR12 Prodrug of claim 69, further comprising a helper lipid.

72) A liposome of claim 71, wherein the helper lipid is set forth in Table II.

73) A solid-lipid nanoparticle (SLNP) comprising the TR12 Prodrug of claim 69.

74) A liposome of claim 70, denoted LNP-TR12.

75) The SLNP of claim 73, denoted SLNP-TR12.

76) The SLNP of claim 73 co-formulated with IC1.

77) The SLNP of claim 73 co-formulated with AR5.

78) The SLNP of claim 73 co-formulated with NTI-47C.

79) The SLNP of claim 73 co-formulated with IC1, wherein the ratio is 8:1 (denoted NTI-121).

80) The SLNP of claim 73 co-formulated with IC1, wherein the ratio is 16:1.

81) A composition comprising a solid-lipid nanoparticle (SLNP) wherein the SLNP further comprises TR12 co-formulated with IC1 (denoted SLNP-TR12-IC1).

82) A composition comprising a solid-lipid nanoparticle (SLNP) wherein the SLNP further comprises TR12 co-formulated with IC1 at a ratio of 8:1 (denoted NTI-121).

83) A composition comprising a solid-lipid nanoparticle (SLNP) wherein the SLNP further comprises TR12 co-formulated with IC1 at a ratio of 16:1 (denoted SLNP-TR12-IC1).

84) A composition comprising a solid-lipid nanoparticle (SLNP) wherein the SLNP further comprises TR12 co-formulated with AR5 (denoted SLNP-TR12-AR5).

85) A composition comprising a solid-lipid nanoparticle (SLNP) wherein the SLNP further comprises TR12 co-formulated with NTI-47C (denoted SLNP-TR12-NTI-47C).

86) The composition of any of claims 79-85 where TR12 comprises the following chemical structure:

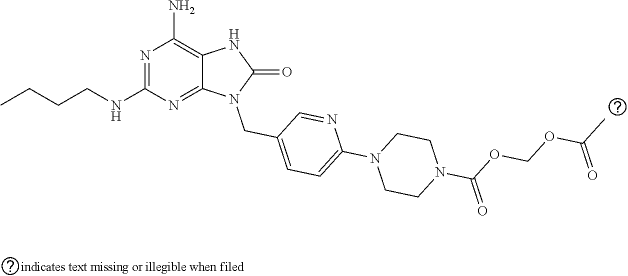 [SMILES: CCCCNc1nc(N)c2[nH]c(=O)n(Cc3ccc(N4CCN(C(=O)OCOC(C)=O)CC4)nc3)c2n1]

87) The composition of any of claims 79-86 used to treat cancer in an individual.

88) The composition of claim 87 used to treat cancer in an individual, wherein the cancer is selected from the group consisting of breast, colon, kidney, melanoma, myeloma, neuroblastoma, liver, lung, pancreatic, prostate, and bladder.

### EXAMPLES

Various aspects of the invention are further described and illustrated by way of the several examples that follow, none of which is intended to limit the scope of the invention.

### Example 1: Chemical Synthesis of TR12 Prodrug Comprising Stearic Acid

Chemical synthesis of a TR12 comprising Stearic Acid was synthesized using the following protocols. Briefly, a solution of compound 1 (77.0 g, 454 mmol, 1.00 eq), compound 1a (88.3 g, 545 mmol, 1.20 eq) and K₂CO₃ (75.3 g, 545 mmol, 1.20 eq) in DMF (500 mL) was stirred at 80° C. for 3 hrs. LCMS (EW29816-9-P1A) showed a substantial desired mass (RT=0.701 min). Next, the organic solvent was evaporated. Then, water (1.00 L) was added, and the mixture was stirred at 20° C. for 0.5 hr. and filtered to get compound 2 (120 g, crude) as a yellow solid. Then, the mixture of compound 2 (120 g, 407 mmol, 1.00 eq) and compound 2a (222 g, 3.04 mol, 300 mL, 7.47 eq) in n-PrOH (900 mL) was stirred at 130° C. for 12 hrs. in autoclave (15 psi). LCMS (EW29816-11-P1A) showed desired mass was detected (RT=0.684 min). The mixture was concentrated. The residue was diluted with water (1.00 L) and extracted with EtOAc (1.00 L*3). The combined organics were washed with brine (1.00 L), dried over Na₂SO₄, filtered, and concentrated. The residue was purified by column chromatography (SiO₂, Dichloromethane/Methanol=100/1−10/1, Dichloromethane/Methanol=10/1, R_{f}=0.57) to give compound 3 (64.0 g, 193 mmol, 47.4% yield) as a yellow solid.

Then, to a solution of compound 3 (63.0 g, 190 mmol, 1.00 eq) in CHCl₃ (400 mL) and MeOH (8.00 mL) was added a solution of Br₂ (45.5 g, 285 mmol, 14.7 mL, 1.50 eq) in CHCl₃ (80 mL) dropwise at 0° C. The mixture was stirred at 0° C. for 2 hrs. Then the mixture was concentrated. Conc. HCl (482 g, 4.96 mol, 473 mL, 37.5% purity, 26.1 eq) was added to the mixture. The mixture was stirred at 100° C. for 3 hrs. LCMS (EW29816-14-P1B) showed the substantial desired mass (RT=0.788 min). The mixture was concentrated. The residue was adjusted to pH=8 with 30% NH₃ H₂O and filtered to get the solid. The crude product was triturated with Petroleum ether/Ethyl acetate=1/1 (200 mL) and filtered to get compound 4 (45.0 g, 129 mmol, 68.1% yield) as a yellow solid.

Then, a mixture of compound 4 (45.0 g, 129 mmol, 1.00 eq) and piperazine (223 g, 2.59 mol, 20.0 eq) was stirred at 140° C. for 2 hrs. LCMS (EW29816-16-P1B) showed the substantial desired mass (RT=0.668 min). HPLC (EW29816-16-P1A1) showed a major peak was detected (RT=1.237 mins). The mixture was concentrated. The residue was purified by prep-HPLC (FA). The organic solvent was evaporated. The aqueous phase was adjusted to pH=8 with saturated Na₂CO₃ solution and filtered to get compound 5 (28.0 g, 70.4 mmol, 54.4% yield) as a white solid.

Then, to a solution of compound 5 (8.00 g, 20.1 mmol, 1.00 eq) in DMF (150 mL) was added compound 12 (9.10 g, 24.1 mmol, 1.20 eq) at 0° C. The mixture was stirred at 20° C. for 12 hrs. LCMS (EW29816-35-P1A) showed the substantial desired product (RT=1.112 mins). The mixture was poured into water (500 mL) and filtered to get the solid. The residue was purified by prep-HPLC (FA) and prep-HPLC (column: Welch Ultimate XB-CN 250*70*10 um;mobile phase: [Heptane-EtOH(0.1% NH3H2O)];B%: 5%-45%, 15 min) (twice) and concentrated to give TR12 (783.5 mg, 1.03 mmol, 5.13% yield, 97.2% purity) as a grey solid, which was confirmed by ¹HNMR, LCMS, and HPLC and TR12 (209.85 mg, 276.11 umol, 1.37% yield, 97.1% purity) as a white solid which was confirmed by ¹HNMR, LCMS, and HPLC. (FIG. 1). The synthesis set forth in this example yields a TR12-Prodrug comprising Stearic Acid with the following chemical structure:

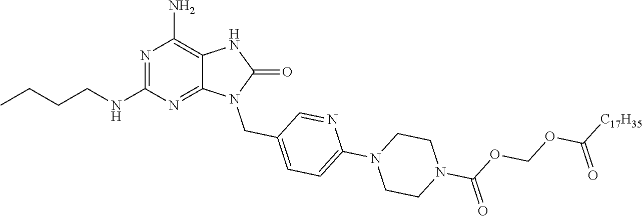

### Example 2: Chemical Synthesis of TR12 and TR13 Prodrug Intermediate(s)

In another experiment, chemical synthesis of the TR12 and TR13 Prodrug intermediates were performed in the following manner. Briefly, to a solution of compound 6 (51.0 g, 396 mmol, 35.2 mL, 1.00 eq) in THF (100 mL) was added compound 7 (24.2 g, 389 mmol, 28.8 mL, 9.83e⁻¹ eq) and Et₃N (40.0 g, 396 mmol, 55.0 mL, 1.00 eq) in THF (100 mL) drop-wise at 0-5° C. The mixture was stirred at 20° C. for 12 hrs. A white solid appeared. The organic solvent was evaporated. The crude product was distilled (−0.1 MPa, 78-84° C.) to give compound 8 (35.0 g, 226 mmol, 57.2% yield) as yellow oil.

Then, a solution of compound 8 (17.0 g, 110 mmol, 1.00 eq) and NaI (24.7 g, 165 mmol, 1.50 eq) in acetone (170 mL) was stirred at 40° C. for 3 hrs. TLC (Ethyl acetate/Petroleum ether=0/1) showed compound 8 was consumed (R_{f}=0.50) and a major spot was detected (R_{f}=0.52). The mixture was filtered, and the filtrate was concentrated. The residue was diluted with Ethyl acetate (300 mL), washed with 10% Na₂SO₃ aqueous solution (200 mL*2), brine (200 mL), dried over Na₂SO₄, filtered, and concentrated to give compound 9 (25.0 g, crude) as yellow oil.

Then, to a solution of compound 10a (34.7 g, 122 mmol, 41.0 mL, 1.20 eq), NaHCO₃ (21.3 g, 254 mmol, 9.88 mL, 2.50 eq) and hydrogen sulfate;tetrabutylammonium (41.4 g, 122 mmol, 1.20 eq) in DCM (150 mL) and H₂O (150 mL) was added compound 9 (25.0 g, 102 mmol, 1.00 eq) in DCM (30 mL) drop-wise. The mixture was stirred at 20° C. for 12 hrs. TLC (Petroleum ether/Ethyl acetate=5/1) showed compound 9 was consumed (R_{f}=0.70) and a major spot was detected (R_{f}=0.64). The mixture was diluted with water (100 mL), extracted with DCM (200 mL*3). The combined organic layers were washed with brine (200 mL), dried over Na₂SO₄, and concentrated. The residue was purified by column chromatography (SiO₂, Petroleum ether/Ethyl acetate=20/1−5/1) to give compound 11 (25.0 g, 62.1 mmol, 61.1% yield) as a white solid.

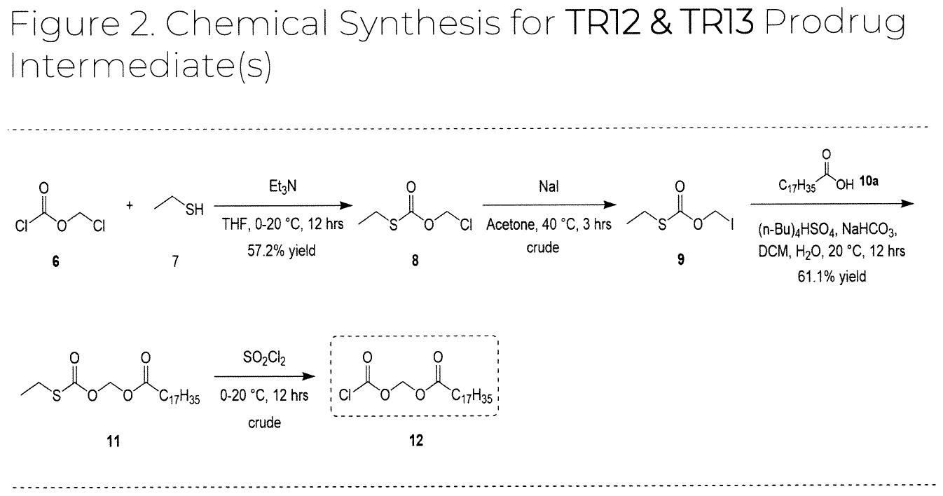

Finally, to compound 11 (15.0 g, 37.3 mmol, 1.00 eq) was added SO₂Cl₂ (50.3 g, 373 mmol, 37.3 mL, 10 eq) drop-wise at 0° C. The mixture was stirred at 20° C. for 12 hrs. TLC (Petroleum ether/Ethyl acetate=10/1) showed compound 11 (R_{f}=0.80) was consumed and a major spot was detected (R_{f}=0.88). The mixture was concentrated to give compound 12 (14.0 g, crude) as yellow oil. (FIG. 2).

### Example 3: Chemical Synthesis of TR13 Prodrug Non-Cleavable Stearic Acid Derivative

In another experiment, chemical synthesis of a TR12 Prodrug comprising non-cleavable Stearic Acid derivative (“denoted TR13”) was synthesized using the following protocols. Briefly, to a solution of compound 10a (4.01 g, 14.1 mmol, 4.74 mL, 0.70 eq), DIEA (7.80 g, 60.4 mmol, 10.5 mL, 3.00 eq), HATU (9.18 g, 24.2 mmol, 1.20 eq) in DMF (10 mL) was added compound 5 (8.00 g, 20.1 mmol, 1.00 eq). The mixture was stirred at 20° C. for 12 hrs. LCMS (EW29816-26-P1A) showed the substantial desired mass (RT=1.053 mins). The mixture was concentrated. The residue was purified by prep-HPLC (FA) and concentrated. The crude product was diluted with MeCN (100 mL) and adjusted to pH=8 with Et₃N. The mixture was filtered to get the solid. The solid was triturated with EtOAc/THF=1/1 (50 mL) and filtered to get TR13 (2.32 g, 3.30 mmol, 16.37% yield, 94.3% purity) as a white solid. (FIG. 3). The synthesis set forth in this example yields a TR12-Prodrug comprising a non-cleavable Stearic Acid derivative (denoted TR13) with the following chemical structure:

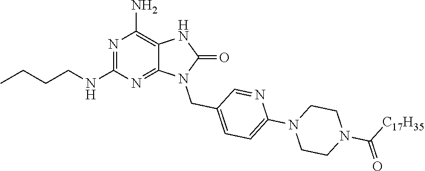

### Example 4: Synthesis and Characterization of SLNP-TR12 Solid-Lipid Nanoparticle

In another experiment, a solid-lipid nanoparticle comprising the TR12 (denoted SLNP-TR12) was synthesized using the following protocol. Briefly, by a solvent diffusion method, with or without, a help of a stabilizer. It is noted that an example of the stabilizer that can be used for this SLNPs include, but are not limited to, Polyvinyl alcohol (e.g., Moliwol 488), poloxamers (e.g., Pluronic F-68, Pluronic F-127), Tween 80 & 20, Kolliphor RH40, etc.

In the first step, a lipid stock solution of DSPC, CHOL, DSPE-PEG, were prepared in ethanol (20 mg/ml). Separately, a TR12 prodrug stock solution was prepared in DMSO (20 mg/ml). For this example, the lipid mixture was obtained by mixing DSPC, CHOL, TR12 and DSPE-PEG at a molar ratio of 34:56:5:5 (with a lipid concentration of 20 mg/ml). This lipid mixture was then heated at 40-45° C. for five (5) minutes. Similarly, the aqueous phase was heated (40-45° C.) using a magnetic hot plate stirrer with constant magnetic stirring (at 300-400 rpm).

Alternatively, in a more simplified method, SLNPS-TR12 also can be synthesized by using only DI water (without stabilizer) in the aqua phase. In this alternative method, the lipid mixture was slowly mixed with this aqueous phase under constant stirring. Once the mixing was completed the entire mixture was sonicate using a water sonicate bath for about ten (10) minutes and then again kept in the magnetic stirrer plate with constant stirring for about another 2-4 hour(s). Finally, the solvent was removed using dialysis membrane of cut off 12 KDa size (Sigma Aldrich) against DI water for at least eight (8) hrs. The Dialysis water was changed at least three (3) times during this time period. The SLNP-TR12 was concentrated according to the need using an Amicon centrifugal filtration device (cut off size 10 KDa, at 3000 g). Alternatively, TFF (Tangential Flow Filtration) system can be used to remove the solvent and concentrate the SLNP-TR12.

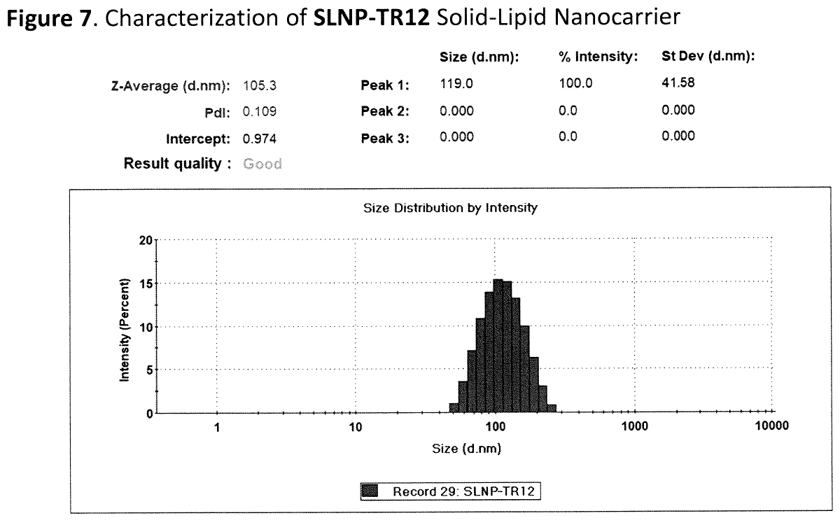

Characterization of the SLNP-TR12 was determined using a Malvern Zetasizer (Malvern Instrumentation Co., Westborough, MA, USA). Briefly, two (2) ml of SLNP-TR12 (concentration 1 mg/ml) was placed in a 4-sided, clear, plastic cuvette and analyzed directly at 25° C. The results shown in FIG. 7 show the Zav size of the nanoparticles were approximately 105.3 nm with a PDI of approximately 0.109.

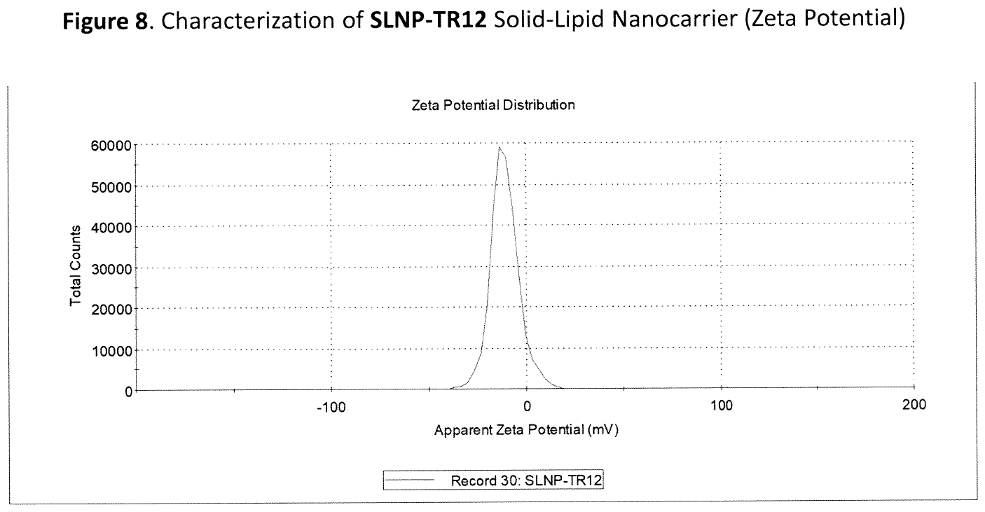

Additionally, Zeta potential of the SLNP-TR12 solid-lipid nanoparticle in aqueous dispersion was determined using a Malvern zeta seizer Instrument (Malvern Instrumentation Co, Westborough, MA, USA). Briefly, approximately one (1) ml of the SLNP (concentration approximately 3 mg/ml in DI water) was placed in a disposable capillary zeta potential cell available for the Zetasizer. The measurement was done at 25° C. The results show the Zeta potential determination of SLNP-TR12 was approximately −10.9 mV (FIG. 8).

### Example 5: Synthesis and Characterization of SLNP-TR12-IC1 (NTI-121) Solid-Lipid Nanoparticle

In another experiment, a solid-lipid nanoparticle comprising the TR12 co-formulated with an immunogenic cell death prodrug (“IC1”) (denoted SLNP-TR12-IC1 and/or NTI-121) was synthesized. It is understood that a solvent diffusion method with or without using a stabilizer may be employed. Example(s) of a stabilizer that can be use is Polyvinyl alcohol (e.g., Moliwol 488), poloxamers (e.g. Pluronic F-68, Pluronic F-127), Tween 80 & 20, Kolliphor RH40, etc. It is understood that different types of SLNPs using different ratios of IC1:TR12 also can be prepared.

In this example, SLNP-IC1-TR12 with a ratio of IC1:TR12 in 8:1 (also denoted NTI-121) was prepared using the solvent diffusion method described above. In the first step, a lipid stock solution of DSPC, CHOL, DSPE-PEG, were prepared in ethanol (20 mg/ml). Also, a stock of IC1 was prepared in ethanol with concentration of 2.5 mg/ml. Separately, a stock solution of TR12 prodrug was prepared in DMSO (20 mg/ml). A lipid mixture was obtained by mixing DSPC, CHOL, IC1, TR12, and DSPE-PEG at a molar ratio of 33:54.56:7:0.44:5 (with a lipid concentration of approximately ˜20 mg/ml). This lipid mixture was then heated at 40-45 degree. The lipid mixture was slowly mixed with this aqueous phase (DI water) under constant stirring. Once the mixing was completed the entire mixture was sonicate using a water sonicate bath for about ten (10) minutes and then again kept in the magnetic stirrer plate with constant stirring for about another 2-4 hour(s).

Finally, the solvent was removed using dialysis membrane of cut off 12 KDa size (Sigma Aldrich) against DI water for at least 8 hrs. The Dialysis water was changed at least 3 times during this time period The SLNP-IC1-TR12 was concentrated according to the need using Amicon centrifugal filtration device (cut off size 10 KDa, at 3000 g). Alternatively, TFF (Tangential Flow Filtration) system can be used to remove the solvent and concentrate the SLNP-IC1-TR12.

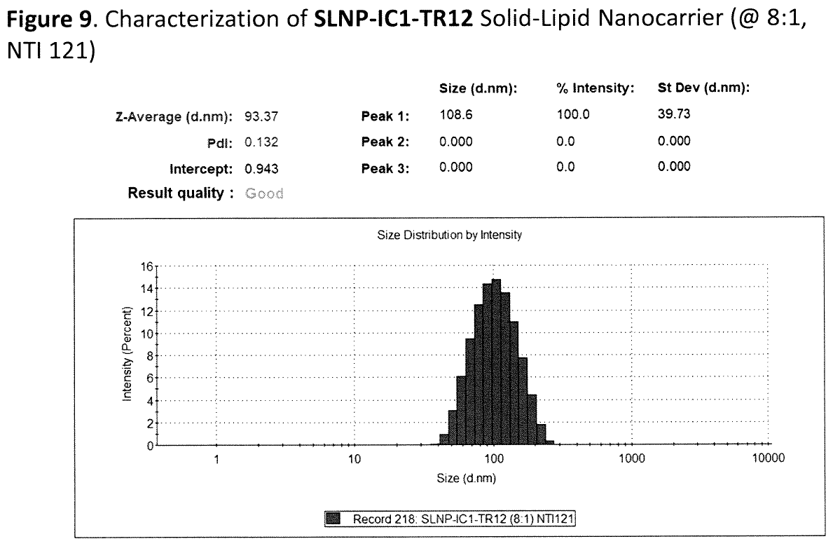

Characterization of the SLNP-TR12-IC1 (NTI-121) was determined using a Malvern Zetasizer (Malvern Instrumentation Co., Westborough, MA, USA). Briefly, two (2) ml of SLNP-IC1-TR12 (concentration 1 mg/ml) was placed in a 4-sided, clear, plastic cuvette and analyzed directly at 25° C. The results shown in FIG. 9 show the Zav size of the nanoparticles were approximately 93.37 nm with a PDI of approximately 0.132.

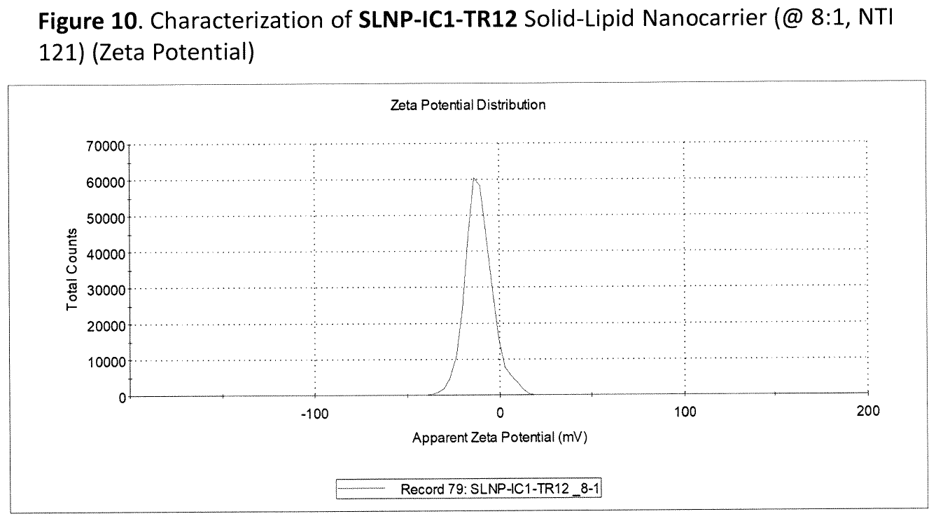

Additionally, Zeta potential of the SLNP-IC1-TR12 (NTI-121) solid-lipid nanoparticle in aqueous dispersion was determined using a Malvern zeta seizer Instrument (Malvern Instrumentation Co, Westborough, MA, USA). Briefly, approximately one (1) ml of the SLNP (concentration approximately 3 mg/ml in DI water) was placed in a disposable capillary zeta potential cell available for the Zetasizer. The measurement was done at 25° C. The results show the Zeta potential determination of SLNP-IC1-TR12 was approximately −12.8 mV (FIG. 10).

### Example 6: Synthesis and Characterization of SLNP-TR12-IC1 (16:1) Solid-Lipid Nanoparticle

In another experiment, a solid-lipid nanoparticle comprising the TR12 co-formulated with an immunogenic cell death prodrug (“IC1”) (denoted SLNP-TR12-IC1) was synthesized. It is understood that a solvent diffusion method with or without using a stabilizer may be employed. Example(s) of a stabilizer that can be use is Polyvinyl alcohol (e.g. Moliwol 488), poloxamers (e.g. Pluronic F-68, Pluronic F-127), Tween 80 & 20, Kolliphor RH40, etc. It is understood that different types of SLNPs using different ratios of IC1:TR12 also can be prepared.

In this example, SLNP-IC1-TR12 with a ratio of IC1:TR12 in 16:1 was prepared using the solvent diffusion method described above. In the first step, a lipid stock solution of DSPC, CHOL, DSPE-PEG, were prepared in ethanol (20 mg/ml). Also, a stock of IC1 was prepared in ethanol with concentration of 2.5 mg/ml. Separately, a stock solution of TR12 prodrug was prepared in DMSO (20 mg/ml). A lipid mixture was obtained by mixing DSPC, CHOL, IC1, TR12, and DSPE-PEG at a molar ratio of 33:54.8:7:0.2:5 (with a lipid concentration of approximately ˜20 mg/ml). This lipid mixture was then heated at 40-45 degree. The lipid mixture was slowly mixed with this aqueous phase (DI water) under constant stirring. Once the mixing was completed the entire mixture was sonicate using a water sonicate bath for about ten (10) minutes and then again kept in the magnetic stirrer plate with constant stirring for about another 2-4 hour(s).

Finally, the solvent was removed using dialysis membrane of cut off 12 KDa size (Sigma Aldrich) against DI water for at least 8 hrs. The Dialysis water was changed at least 3 times during this time period The SLNP-IC1-TR12 was concentrated according to the need using Amicon centrifugal filtration device (cut off size 10 KDa, at 3000 g). Alternatively, TFF (Tangential Flow Filtration) system can be used to remove the solvent and concentrate the SLNP-IC1-TR12 (16:1).

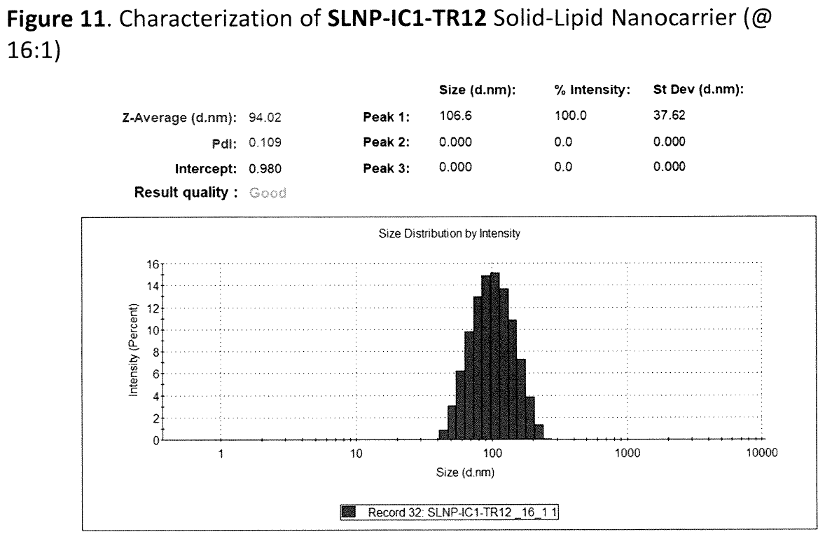

Characterization of the SLNP-TR12-IC1 was determined using a Malvern Zetasizer (Malvern Instrumentation Co., Westborough, MA, USA). Briefly, two (2) ml of SLNP-IC1-TR12 (concentration 1 mg/ml) was placed in a 4-sided, clear, plastic cuvette and analyzed directly at 25° C. The results shown in FIG. 11 show the Zav size of the nanoparticles were approximately 94.02 nm with a PDI of approximately 0.109.

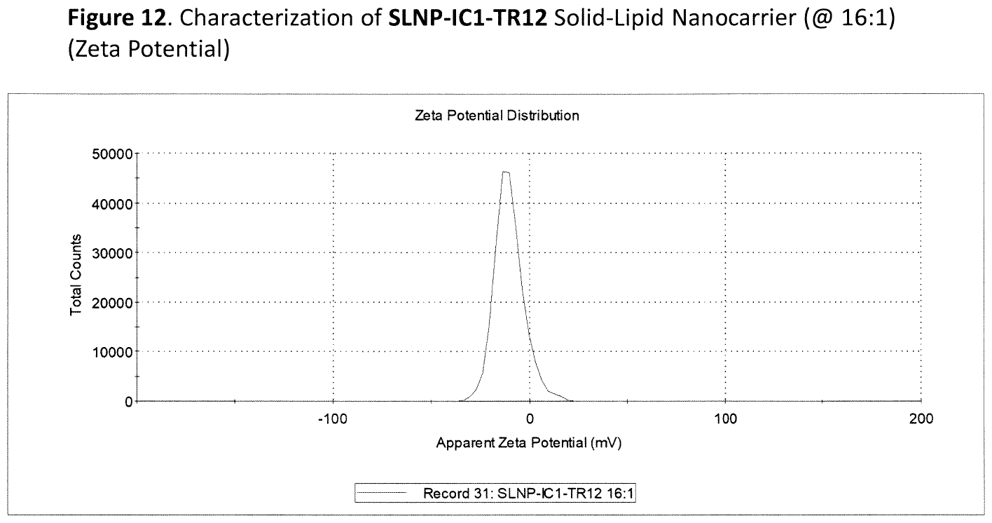

Additionally, Zeta potential of the SLNP-IC1-TR12 solid-lipid nanoparticle in aqueous dispersion was determined using a Malvern zeta seizer Instrument (Malvern Instrumentation Co, Westborough, MA, USA). Briefly, approximately one (1) ml of the SLNP (concentration approximately 3 mg/ml in DI water) was placed in a disposable capillary zeta potential cell available for the Zetasizer. The measurement was done at 25° C. The results show the Zeta potential determination of SLNP-IC1-TR12 was approximately −10.9 mV (FIG. 12).

### Example 7: Synthesis and Characterization of SLNP-TR12-AR5 Solid-Lipid Nanoparticle

In another experiment, a solid-lipid nanoparticle comprising the TR12 co-formulated with an A2aR inhibitor (“AR5”) (denoted SLNP-TR12-AR5). The combination Solid lipid nanoparticles of AR5 and TR12 was prepared by solvent diffusion method using different types of stabilizers as described above. As previously stated, these SLNPs can be prepared without any stabilizer as well.

In the first step, a lipid stock solution of DSPC, CHOL, DSPE-PEG, was prepared in ethanol (20 mg/ml). Separately, a AR5 and TR12 prodrug stock solution was prepared in DMSO (20 mg/ml). A lipid mixture was obtained by mixing DSPC, CHOL, AR5, TR12, and DSPE-PEG at a molar ratio of 26:39:28:1:5 (with a lipid concentration of 20 mg/ml). This lipid mixture was then heated at 45-50 degree C. Similarly, the aqueous phase containing the appropriate stabilizer (e.g., 1-5% w/v Pluronic F127) was heated using a magnetic hot plate stirrer with constant magnetic stirring (at 300-400 rpm). The lipid mixture was slowly mixed with this aqueous phase under constant stirring. Once the mixing was completed the entire mixture was sonicate using a water sonicate bath for about 10 minutes and then again kept in the magnetic stirrer plate with constant stirring for about another one (1) hour.

Finally, the solvent was removed using dialysis membrane of cut off 12 KDa size (Sigma Aldrich) against DI water for at least 8 hrs. The Dialysis water was changed at least 3 times during this time period. The SLNP-AR5-TR12 was concentrated according to the need using Amicon centrifugal filtration device (cut off size 10 KDa, at 3000 g). Alternatively, TFF (Tangential Flow Filtration) system can be used to remove the solvent and concentrate this SLNPs.

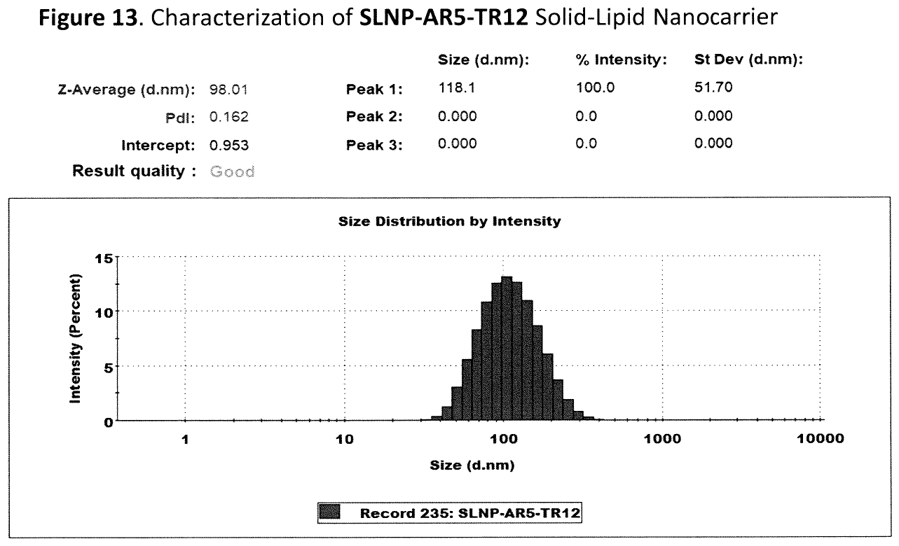

Characterization of the SLNP-TR12-AR5 was determined using a Malvern Zetasizer (Malvern Instrumentation Co., Westborough, MA, USA). Briefly, two (2) ml of SLNP-TR12-AR5 (concentration 1 mg/ml) was placed in a 4-sided, clear, plastic cuvette and analyzed directly at 25° C. The results shown in FIG. 13 show the Zav size of the nanoparticles were approximately 98 nm with a PDI of approximately 0.162.

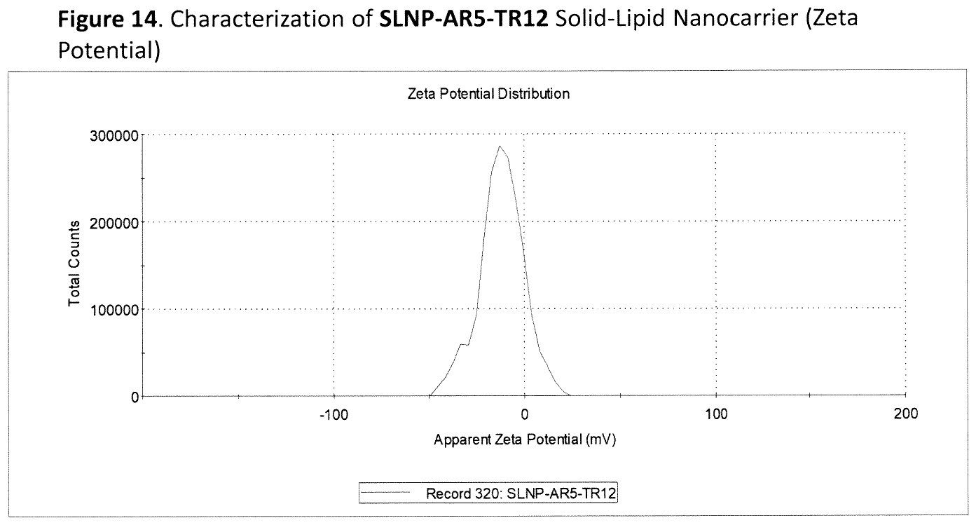

Additionally, Zeta potential of the SLNP-TR12-AR5 solid-lipid nanoparticle in aqueous dispersion was determined using a Malvern zeta seizer Instrument (Malvern Instrumentation Co, Westborough, MA, USA). Briefly, approximately one (1) ml of the SLNP (concentration approximately 3 mg/ml in DI water) was placed in a disposable capillary zeta potential cell available for the Zetasizer. The measurement was done at 25° C. The results show the Zeta potential determination of SLNP-TR12-AR5 was approximately −12.0 mV (FIG. 14).

### Example 8: Synthesis and Characterization of SLNP-TR12-NTI-47C Solid-Lipid Nanoparticle

In another experiment, a solid-lipid nanoparticle comprising the TR12 co-formulated with NTI-47C, a custom peptide (GSGCERVIGTGWVRC) (SEQ ID NO: 1) conjugated to Palmitoyl (“NTI-47C”) (denoted SLNP-TR12-NTI-47C) was synthesized. By using this peptide (CERVIGTGWVRC) (SEQ ID NO: 2) in this type of SLNP, it is contemplated that function-blocking peptide (CERVIGTGWVRC) (SEQ ID NO: 2) structurally mimics an epitope on CD47 and binds to SIRPα. It is understood that the CD47 molecule is well known as a widely expressed cellular surface receptor activating the transduction of the “don't-eat-me” signal. Thereby, it can decrease the wanted uptake of the nanoparticles by macrophages and has the potential to stay in blood circulation for longer time.

SLNP-TR12-NTI-47C was synthesized using the following protocol. Briefly, by solvent diffusion and using various types of stabilizers as previously described (e.g., Polyvinyl alcohol (e.g., Moliwol 488), poloxamers (e.g., Pluronic F-68, Pluronic F-127), Tween 80 & 20, and Kolliphor RH40, etc.)) may be used as a stabilizer to synthesize SLNP-TR12-NTI-47C. Briefly, in the first step, a lipid stock solution of DSPC, CHOL, and NTI-47C, was prepared in ethanol (20 mg/ml). Separately, a TR12 prodrug stock solution was prepared in DMSO (20 mg/ml). A lipid mixture was obtained by mixing DSPC, CHOL, NTI-47C, TR12, and DSPE-PEG at a molar ratio of 29:35:31:5 (with a lipid concentration of 20 mg/ml). This lipid mixture was then heated at 40-45 degree centigrade for approximately five (5) min. Similarly, the aqueous phase containing the appropriate stabilizer (e.g., 2% w/v Pluronic F127) was heated using a magnetic hot plate stirrer with constant magnetic stirring (at 300-400 rpm). The lipid mixture was slowly mixed with this aqueous phase under constant stirring. Once the mixing was completed the entire mixture was sonicate using a water sonicate bath for about ten (10) minutes and then again kept in the magnetic stirrer plate with constant stirring for about another 1 hour. Finally, the solvent was removed using dialysis membrane of cut off 12 KDa size (Sigma Aldrich) against DI water for at least 6 hrs. The dialysis water was changed at least three (3) times during this time period. The SLNP-TR12-NTI-47C was concentrated according to the need using Amicon centrifugal filtration device (cut off size 10 KDa, at 3000 g).

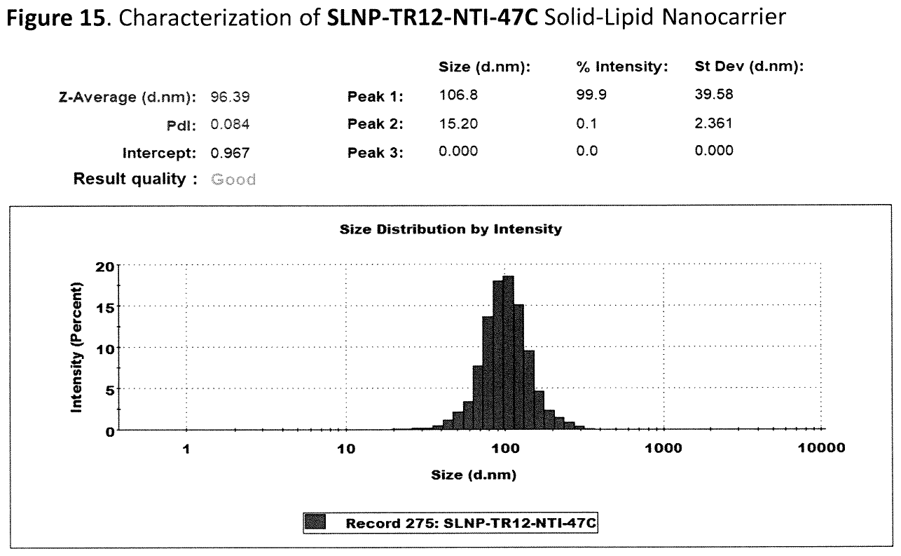

Characterization of the SLNP-TR12-NTI-47C was determined using a Malvern Zetasizer (Malvern Instrumentation Co., Westborough, MA, USA). Briefly, two (2) ml of SLNP-TR12-NTI-47C (concentration 1 mg/ml) was placed in a 4-sided, clear, plastic cuvette and analyzed directly at 25° C. The results shown in FIG. 15 show the Zav size of the nanoparticles were approximately 96.4 nm with a PDI of approximately 0.084.

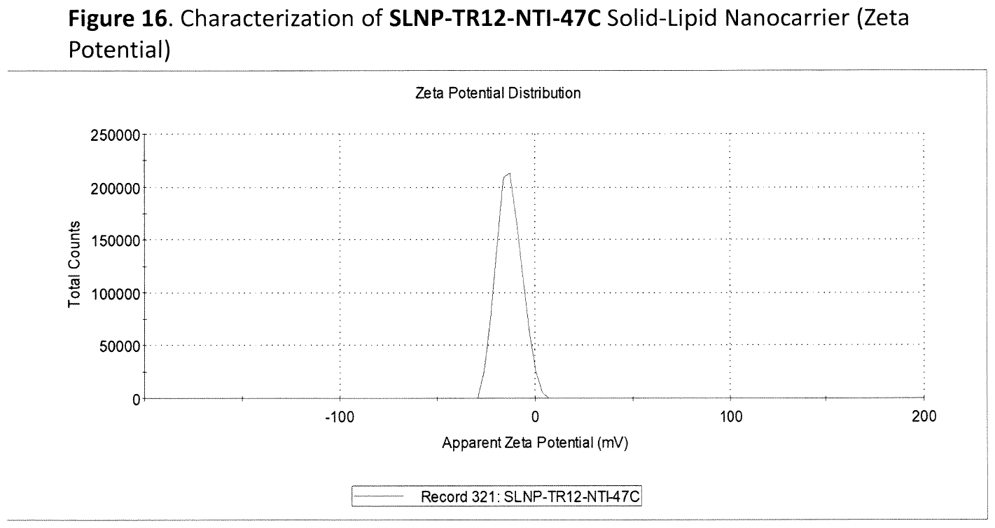

Additionally, Zeta potential of the SLNP-TR12-NTI-47C solid-lipid nanoparticle in aqueous dispersion was determined using a Malvern zeta seizer Instrument (Malvern Instrumentation Co, Westborough, MA, USA). Briefly, approximately one (1) ml of the SLNP (concentration approximately 3 mg/ml in DI water) was placed in a disposable capillary zeta potential cell available for the Zetasizer. The measurement was done at 25° C. The results show the Zeta potential determination of SLNP-TR12-NTI-47C was approximately −13.3 mV (FIG. 16).

### Example 9: Tumor Inhibition of SLNP-TR12 as a Single Agent Using EMT6 Cells In Vivo

Evaluation of SLNP-TR12 as a single agent was performed using the following protocols. Briefly, murine breast cancer EMT6 cells (0.5×10⁶) were inoculated subcutaneously in the right rear flank region of Balb/c mice. Animals were treated with vehicle control and SLNP-TR12 at 1 mg/kg BIW through i.v. injection. Tumor volumes were measured three (3) times in two dimensions using a caliper, and the volume was calculated using the formula: V=(L×W×W)×0.5, where V is tumor volume, L is tumor length (the longest tumor dimension), and W is tumor width (the longest tumor dimension perpendicular to L).

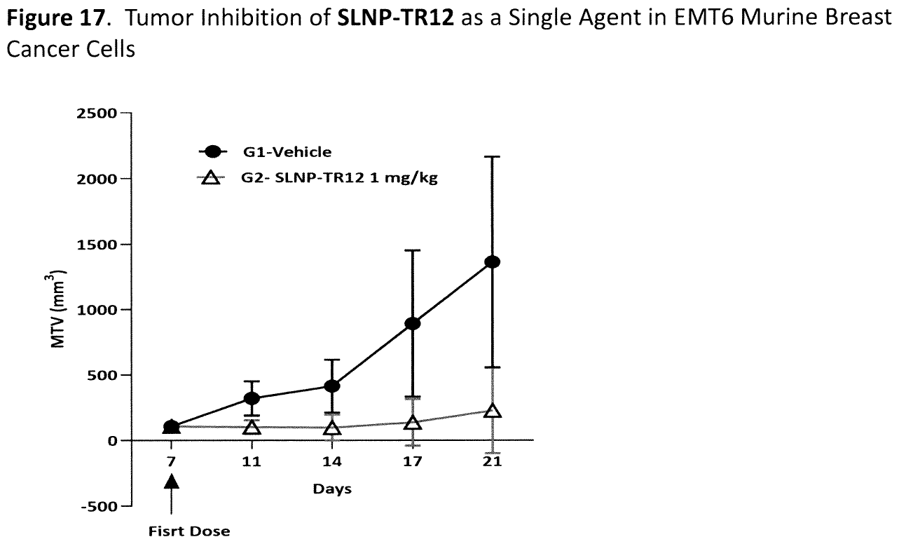

The results show treatment with SLNP-TR12 at 1 mg/kg produced significant tumor growth inhibition when compared to the vehicle control. (See, FIG. 17).

### Example 10: Tumor Inhibition of SLNP-TR12 in Various Doses Compared Against SLNP-TR5 in Various Doses Using EMT6 Cells In Vivo

Evaluation of SLNP-TR12 compared to SLNP-TR5 in various doses was performed using the following protocols. Briefly, murine breast cancer EMT6 cells (0.5×10⁶) were inoculated subcutaneously in the right rear flank region of Balb/c mice. Animals were treated with vehicle control and SLNP-TR12 or SLNP-TR5 (Telratolimod in Solid Nanoparticle form) at 0.5 or 1 mg/kg BIW through i.v. injection. Tumor volumes were measured three (3) times in two dimensions using a caliper, and the volume was calculated using the formula: V=(L×W×W)×0.5, where V is tumor volume, L is tumor length (the longest tumor dimension), and W is tumor width (the longest tumor dimension perpendicular to L).

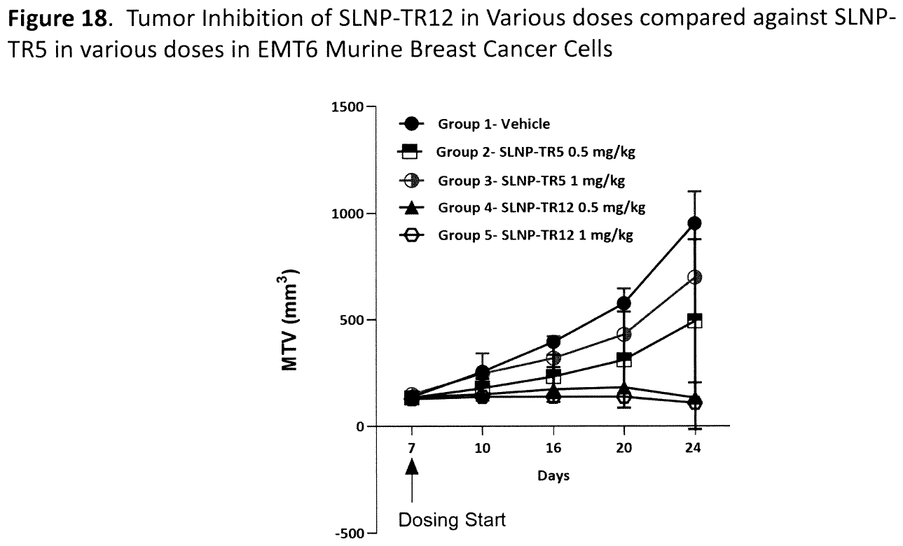

The result shows treatment of SLNP-TR12 at either 0.5 mg/kg or 1 mg/kg produced significant tumor growth inhibition. In addition, the results show that the tumor growth inhibition was greater that SLNP-TR5 at the identical dose. (See, FIG. 18).

### Example 11: Tumor Inhibition of SLNP-TR12 in Combination with SLNP-IC1 Using EMT6 Cells In Vivo

Evaluation of SLNP-TR12 in combination with SLNP-IC1 was performed using the following protocols. Briefly, murine breast cancer EMT6 cells (0.5×10⁶) were inoculated subcutaneously in the right rear flank region of Balb/c mice. Animals were treated with vehicle control and SLNP-TR12 at 0.5 mg/kg, SLNP-IC1 (Doxorubicin-HCL-Stearic Acid Solid Nanoparticle form) at 2 mg·kg or SLNP-IC1-TR12 at 2/0.5 mg/kg BIW through i.v. injection. Tumor volumes were measured three (3) times in two dimensions using a caliper, and the volume was calculated using the formula: V=(L×W×W)×0.5, where V is tumor volume, L is tumor length (the longest tumor dimension), and W is tumor width (the longest tumor dimension perpendicular to L).

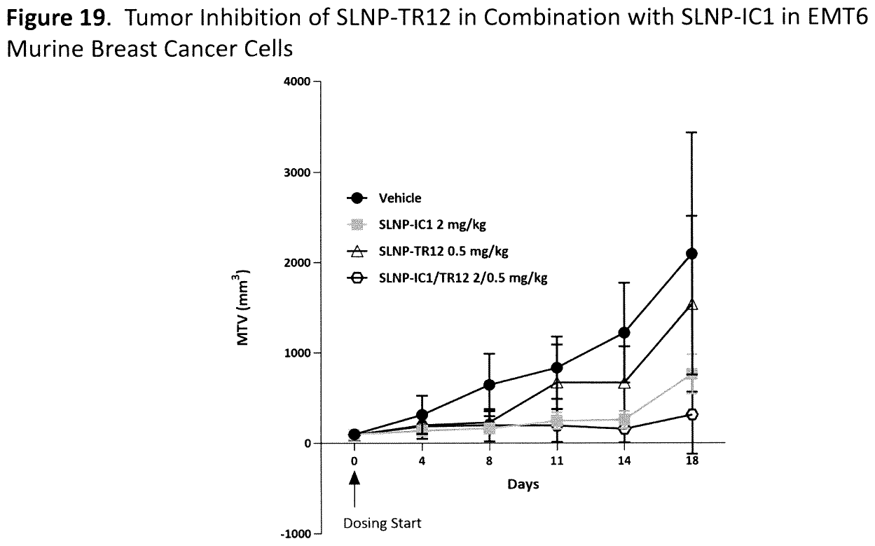

The result shows treatment with combination of SLNP-IC1 and SLNP-TR12 induced significant tumor growth inhibition. (See, FIG. 19).

### Example 12: Tumor Inhibition of SLNP-TR12 in Combination with SLNP-IC1 Using EMT6 Cells In Vivo

In another experiment, Evaluation of SLNP-TR12 in combination with SLNP-IC1 was performed using the following protocols. Briefly, murine breast cancer EMT6 cells (0.5×10⁶) were inoculated subcutaneously in the right rear flank region of Balb/c mice. Animals were treated with vehicle control and SLNP-TR12 at 0.5 mg/kg, SLNP-IC1 (Doxorubicin-HCL-Stearic Acid in SLNP form) at 2 mg/kg or SLNP-IC1-TR12 at 2/0.5 mg/kg BIW through i.v. injection. Tumor volumes were measured three (3) times in two dimensions using a caliper, and the volume was calculated using the formula: V=(L×W×W)×0.5, where V is tumor volume, L is tumor length (the longest tumor dimension), and W is tumor width (the longest tumor dimension perpendicular to L).

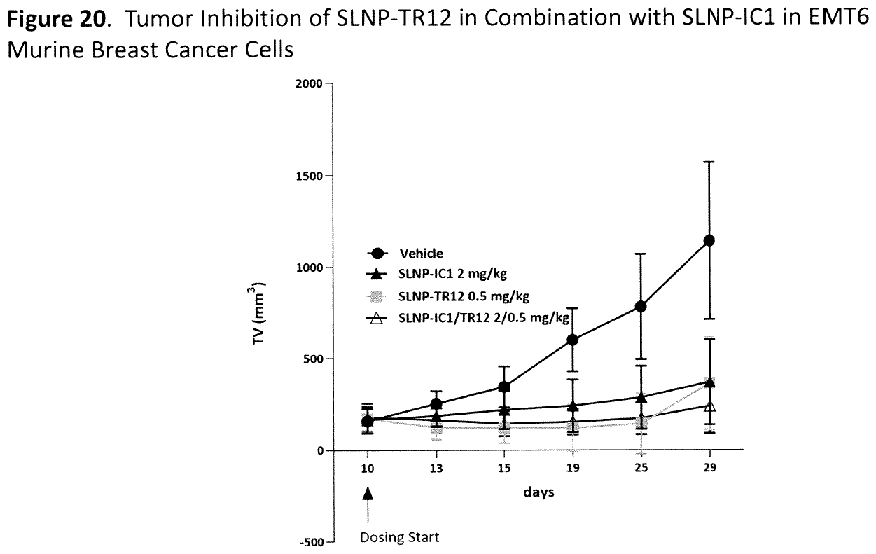

The result shows treatment with combination of SLNP-IC1 and SLNP-TR12 induced significant tumor growth inhibition. (See, FIG. 20).

### Example 13: In Vitro Validation of TR12 Prodrug in Solid-Lipid Nanoparticle (“SLNP”) Form Mechanism of Action

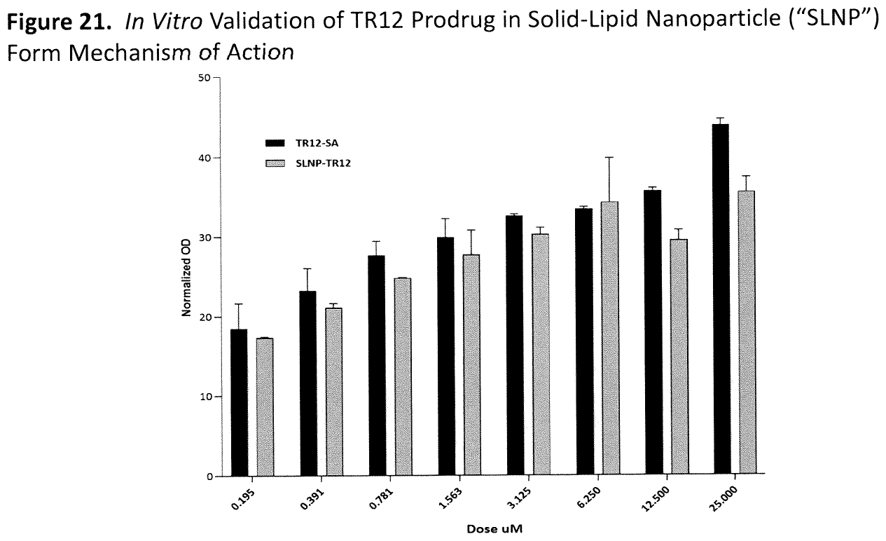

To confirm that SLNP-TR12 possesses biological effects in-vitro and also targets TLR7, the following protocols were used. By way of background, RAW-Blue™ Cells and QUANTI-Blue™ (InvivoGen, San Diego, CA) assay were utilized. Raw Blue™ express human Toll-like receptors (“TLRs”) and an NF-κB/AP-1-inducible SEAP (secreted embryonic alkaline phosphatase) reporter gene. Stimulation of these cells with TR12 leads to NF-κB activation through TLR7 which can be measured by detection of SEAP levels. Briefly, RAW-Blue™ Cells were incubated with TR12-SA (TR12-Stearic Acid) and SLNP-TR12 (TR12 Stearic Acid in Solid Lipid Nanoparticle from) at different concentration. After 24 h incubation with the compounds, TLR stimulation was assessed by measuring the levels of SEAP optimal density (OD) using QUANTI-Blue™ assay. ODs were normalized to the control (untreated) group. The results showed that treating the cells with TR12 prodrug and SLNP-TR12 causes stimulation of TLR-7 confirming the mechanism of action of TR12 and the activity of TR12 in SLNP form. (See, FIG. 21).

### Example 14: In Vitro Validation of SLNP-TR12 Compared to SLNP-TR5 Mechanism of Action

In another experiment, in vitro biological activity of SLNP-TR12 compared to SLNP-TR5 was confirmed using the following experiment(s). By way of background, to confirm SLNP-TR12 can have biological effects in-vitro a RAW-Blue™ Cells and QUANTI-Blue™ (InvivoGen, San Diego, CA) assay was used. It is noted that Raw Blue™ express human TLRs and an NF-κB/AP-1-inducible SEAP (secreted embryonic alkaline phosphatase) reporter gene. Stimulation of these cells with TR12 and TR5 can lead to NF-κB activation through TLR7 or TLR7/8 which can be measured by detection of SEAP levels.

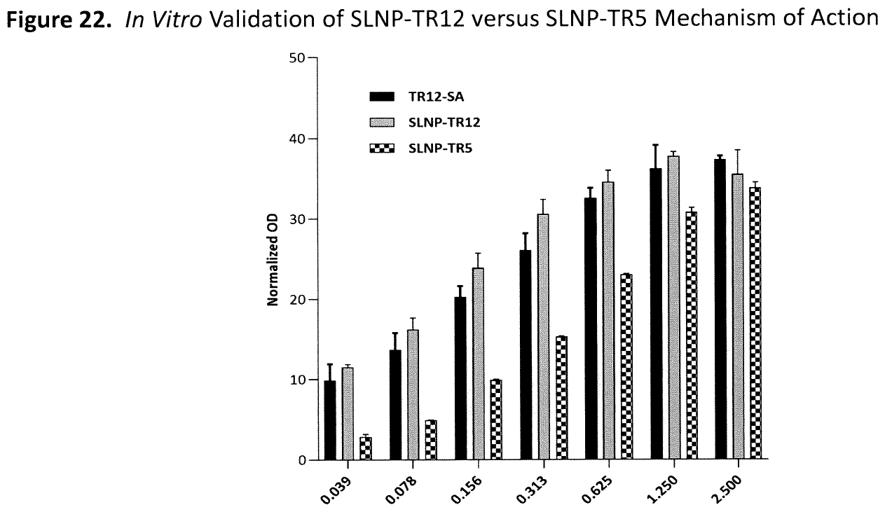

Briefly, RAW-Blue™ Cells were incubated with TR12-SA (TR12-Stearic Acid), SLNP-TR12 (TR12-Stearic Acid in Solid Lipid Nanoparticle from) or SLNP-TR5 (Telratolimod in Solid Lipid Nanoparticle from at different concentration. After 24 h incubation with the compounds, TLR stimulation was assessed by measuring the levels of SEAP optimal density (OD) using QUANTI-Blue™ assay. ODs were normalized to the control (untreated) group. The results showed that treating the cells with TR12 causes stimulation of TLR7 confirming the mechanism of action of TR12 and its activity in SLNP form. Additionally, the SLNP-TR12 showed higher potency when compared to SLNP-TR5. (See, FIG. 22).

### Example 15: In Vitro Validation of SLNP-TR12 Specificity to Toll-like Receptor 7 (TLR7)

In another experiment, in vitro biological activity of SLNP-TR12 specificity to TLR7 was confirmed using the following experiment. By way of background, to confirm that TR12 is specific to TLR7, and does not target TLR8, a RAW-Blue™ and HEK-Blue™ hTLR8 cell lines, and QUANTI-Blue™ (InvivoGen, San Diego, CA) assay was used. It is noted that despite Raw-Blue cells that express a variety of TLRs including TLR7 and TLR8, the HEK-Blue™ hTLR8 cells dominantly express the human TLR8 gene and an NF-κB/AP-1-inducible SEAP (secreted embryonic alkaline phosphatase) reporter gene. Thus, SEAP levels produced upon TLR8 stimulation can be readily determined by performing the assay in HEK-Blue™ Detection.

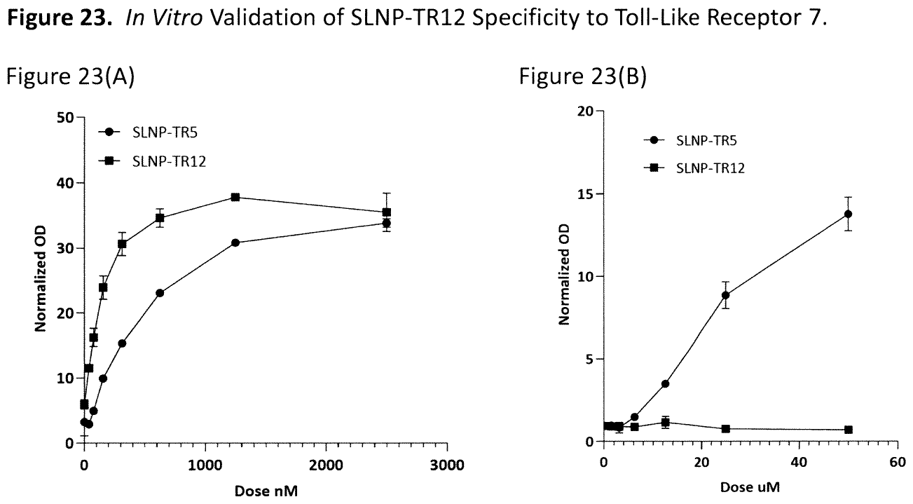

Briefly, RAW-Blue™ Cells were incubated with SLNP-TR5 or SLNP-TR12 at different concentration. After 24 h incubation with the compounds, TLR stimulation was assessed by measuring the levels of SEAP optimal density (OD) using QUANTI-Blue™ assay. ODs were normalized to the control (untreated) group (See, FIG. 23(A)).

Concurrently, HEK-Blue TLR8 were incubated with SLNP-TR5 or SLNP-TR12 at different concentrations. After 24 h incubation with the compounds, TLR stimulation was assessed by measuring the levels of SEAP optimal density (OD) using QUANTI-Blue™ assay. ODs were normalized to the control (untreated) group (See, FIG. 23(B)).

Taken together, the results showed both TR5 and TR12 prodrug in SLNP form stimulated TLRs in RAW-Blue cells, and it is notable that SLNP-TR12 had higher potency. The EC50s for SLNP-TR5 and SLNP-TR12 were calculated at 339.7 and 77.47 nM, respectively.

However, when HEK-Blue hTLR8 cells were treated with SLNP-TR5 and SLNP-TR12 only SLNP-TR5 was able to stimulate the cells. No SEAP induction was seen in the cells treated with SLNP-TR12. The potency of SLNP-TR5 in stimulating the TLR-7 pathway in HEK-Blue hTLR8 cells was lower than in Raw-Blue cells (0.33 vs 21.69 uM). Accordingly, since TR5 is a TLR7/8 agonist and TR12 is TLR7 agonist, the results showed that while TR5 stimulates both TLR7 and TLR8, TR12 is specific to TLR7 but not TLR8. (See, FIG. 23).

### Example 16: Ex Vivo Validation of SLNP-TR12 Activity

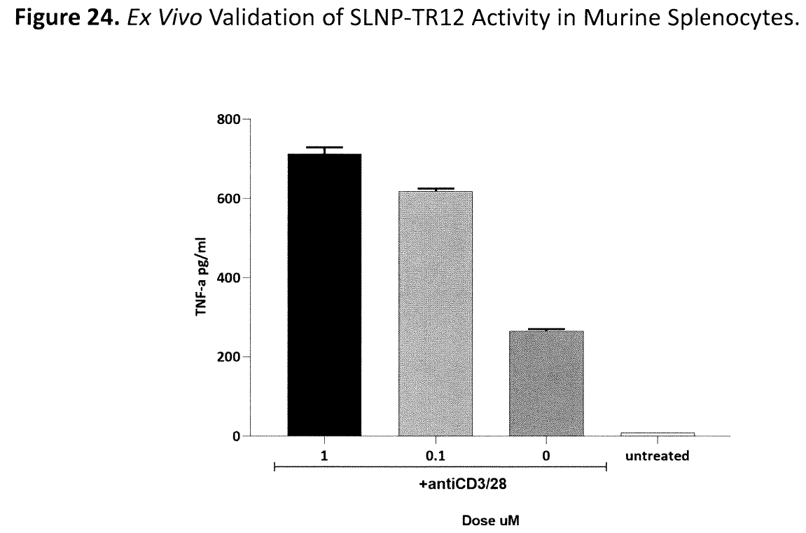

In another experiment, ex vivo biological activity of SLNP-TR12 was confirmed using the following experiment. Briefly, splenocytes were isolated from Balb/c mice. Approximately, 1×10⁶ million cells (2×10⁶/ml) were cultured in media. Cells were then treated with antiCD3/28 cell activator beads at a cell:bead ratio of 1:1. The samples were then treated with different concentration of SLNP-TR12. After 24 hours supernatant was collected and analyzed for TN F-alpha by ELISA. The results showed SLNP-TR12 can induce TNF-alpha secretion in Splenocytes isolated from mice. (See, FIG. 24).

### Example 17: Ex Vivo Validation of SLNP-TR12 Activity

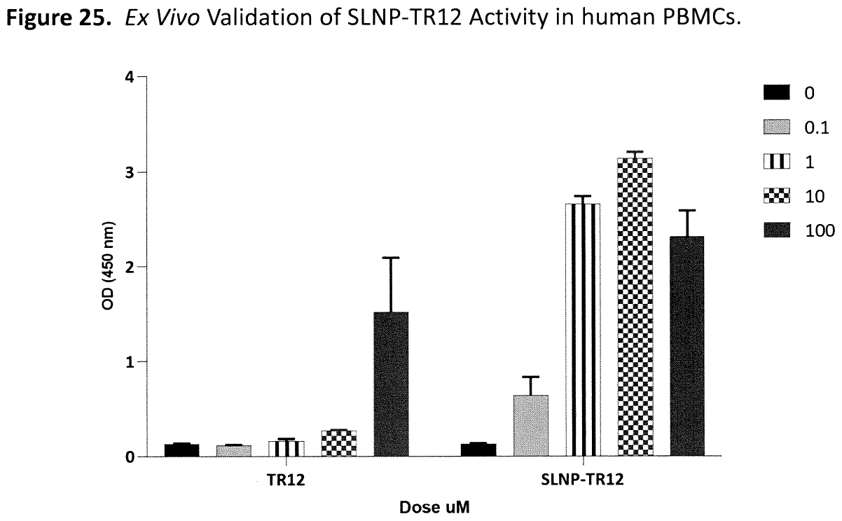

In another experiment, ex vivo biological activity of SLNP-TR12 was confirmed using the following experiment. Briefly, Human Peripheral Blood Mononuclear Cells (PBMCs) were treated with different concentration of TR-12 Prodrug and SLNP-TR12. After 24 hours supernatant was collected and analyzed for TNF-alpha by ELISA. The results showed SLNP-TR12 can induce TNFa secretion in human PBMCs. (See, FIG. 25).

### Example 18: In Vivo Validation of SLNP-TR12 Efficacy in EMT-6 Tumor Model

In another experiment, in vivo efficacy/activity of SLNP-TR12 was confirmed using the following experiment(s). Briefly, murine breast cancer EMT6 cells (0.5×10⁶) were inoculated subcutaneously in the right rear flank region of Balb/c mice. Animals were treated with vehicle control and SLNP-TR12 at 0.25 and 0.125 mg/kg bi-weekly through iv injection. Tumor volumes were measured three (3) times in two dimensions using a caliper, and the volume was calculated using the formula: V=(L×W×W)×0.5, where V is tumor volume, L is tumor length (the longest tumor dimension), and W is tumor width (the longest tumor dimension perpendicular to L).

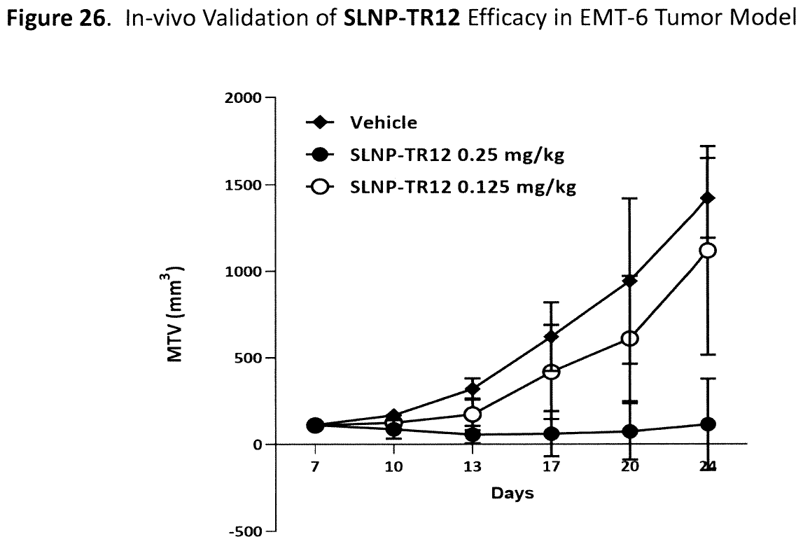

The result show that treatment with 0.25 mg/kg of SLNP-TR12 induces significant tumor growth inhibition. However, the 0.125 mg/kg treatment(s) does not induce significant tumor growth inhibition. (See, FIG. 26).

### Example 19: In Vivo Validation of SLNP-TR12 Efficacy in 4T-1 Tumor Model

In another experiment, in vivo efficacy/activity of SLNP-TR12 was confirmed using the following experiment(s). Briefly, murine breast cancer 4T-1 cells (0.3×10⁶) were inoculated subcutaneously in the right rear flank region of Balb/c mice. Animals were treated with vehicle control and SLNP-TR12 at 0.5 mg/kg bi-weekly through i.v injection. Tumor volumes were measured three (3) times in two dimensions using a caliper, and the volume was calculated using the formula: V=(L×W×W)×0.5, where V is tumor volume, L is tumor length (the longest tumor dimension), and W is tumor width (the longest tumor dimension perpendicular to L).

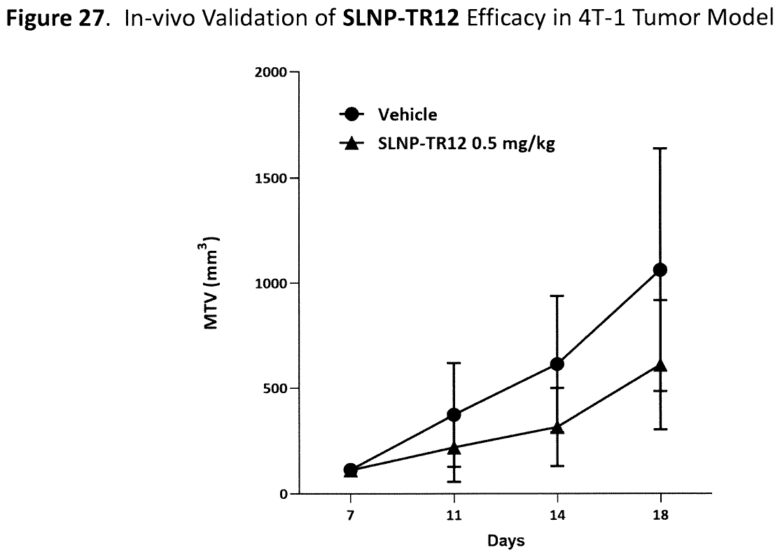

The result shows treatment with 0.5 mg/kg of SLNP-TR12 induces tumor growth inhibition in 4T-1 model. (See, FIG. 27).

### Example 20: In Vivo Validation of Multiple Doses of TR12 Prodrug (SLNP-TR12) Alone and in Combination with IC1 Prodrug (SLNP-IC1) Efficacy in EMT-6 Tumor Model

In another experiment, in vivo efficacy/activity of SLNP-TR12 as a single agent and/or in combination with SLNP-IC1 was confirmed using the following experiment(s). Briefly, murine breast cancer EMT-6 cells (0.5×10⁶) were inoculated subcutaneously in the right rear flank region of Balb/c mice. Animals were treated with vehicle control and SLNP-TR12 at 0.25 (HD) or 0.125 (LD) mg/kg, SLNP-IC1 at 2 (HD) or 1 (LD) mg/kg, SLNP-IC1/TR12 at 2/0.25 (HD) or 1/0.125 (LD) mg/kg, bi-weekly through iv injection. Additionally, in one group the animals were treated with SLNP-IC1/TR12 (HD) via ip injection.

Tumor volumes were measured three (3) times in two dimensions using a caliper, and the volume was calculated using the formula: V=(L×W×W)×0.5, where V is tumor volume, L is tumor length (the longest tumor dimension), and W is tumor width (the longest tumor dimension perpendicular to L).

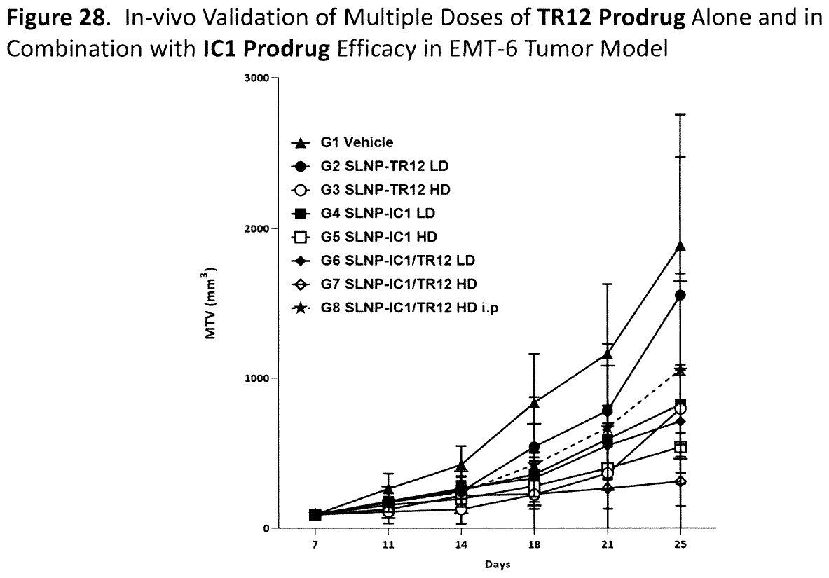

The result showed that the combination treatment with IC1 and TR12 at high doses (HD) induced the most significant tumor growth inhibition. Notably, ip injection of SLNP-IC1/TR12 did induce tumor growth inhibition, however, the iv-injection method of delivery was shown to be more efficient compared to the ip method of delivery. (See, FIG. 28).

### Example 21: Maximum Tolerated Dose (MTD) of Doxorubicin Prodrug Alone and in Combination with SLNP-TR12 in Balb/c Mouse Model

In another experiment, the MTD of SLNP-TR12 as a single agent and/or in combination with SLNP-IC1 was confirmed using the following experiment(s). Briefly, Balb/c mice were treated with variable doses of SLNP-IC1 (Doxorubicin Prodrug in Solid Lipid Nanoparticle form), SLNP-TR12, and a combination of SLNP-IC1 and SLNP-TR12 via iv injection.

Animals were observed for the clinical signs of toxicity including but not limited to, orbital tightening, ear position, nose bulging, lethargy, and ruffled fur, at 30 minutes and 4 hours post-injection. Animals' body weight was measured at 24-, 48-, and 72-hours post-injection.

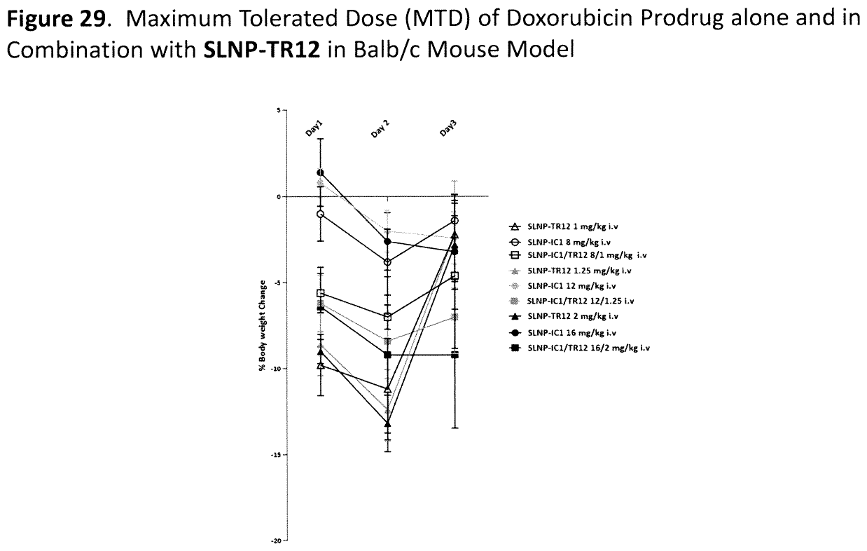

The results show no immediate sign of toxicity was observed in any of the groups. SLNP-IC1 as a single agent caused minor body weight loss. Notably, SLNP-TR12 as a single agent induced moderate to severe body weight loss by 48 hours post-injection and after 48 hours the body weight loss recovered with no intervention. A combination of SLNP-IC1 and SLNP-TR12 induced moderate body weight loss which was lower than SLNP-TR12 treated groups. (See, FIG. 29).

### Example 22: In Vivo Validation of SLNP-IC1-TR12 Efficacy in B16F10 Melanoma Tumor Model

In another experiment, in vivo efficacy/activity of SLNP-IC1-TR12 was confirmed using the following experiment(s). Briefly, murine melanoma B16F10 cells (cells (0.2×10⁶) were inoculated subcutaneously in the right rear flank region of B57/BL mice. Animals were treated with vehicle control or SLNP-IC1-TR12 at 2.0/0.25 mg/kg two (2) times a week through iv injection. Tumor volumes were measured three (3) times in two dimensions using a caliper, and the volume was calculated using the formula: V=(L×W×W)×0.5, where V is tumor volume, L is tumor length (the longest tumor dimension), and W is tumor width (the longest tumor dimension perpendicular to L). Body weight was measured three times a week. The tumor growth inhibition (TGI) was calculated based on the tumor size data of day 9.

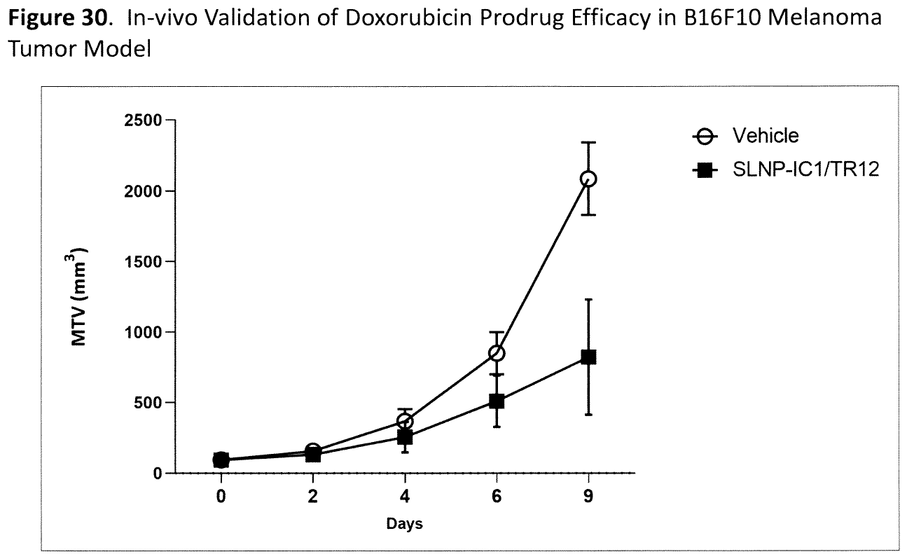

The results show, treatment with SLNP-IC1-TR12 for six (6) doses for 2 weeks produces anti-tumor activity when compared with the vehicle-treated group. The TGI was at 63.45% (See, FIG. 30).

### Example 23: In Vivo Validation of SLNP-IC1-TR12 Efficacy in MPC11 Multiple Myeloma Tumor Model

In another experiment, in vivo efficacy/activity of SLNP-IC1-TR12 was confirmed using the following experiment(s). Briefly, murine multiple myeloma MPC11 cells (0.2×10⁶) were inoculated subcutaneously in the right flank region of Balb/c mice. Animals were treated with vehicle control or SLNP-IC1-TR12 at 2.0/0.25 mg/kg, two (2) times a week through iv injection. Tumor volumes were measured three (3) times in two dimensions using a caliper, and the volume was calculated using the formula: V=(L×W×W)×0.5, where V is tumor volume, L is tumor length (the longest tumor dimension), and W is tumor width (the longest tumor dimension perpendicular to L). Body weight was measured three times a week. The tumor growth inhibition (TGI) was calculated based on the tumor size data of day 9.

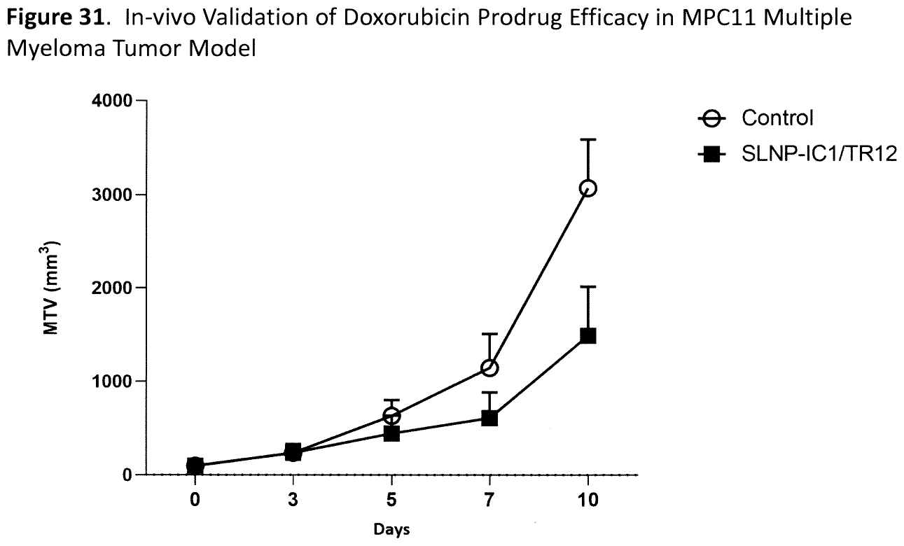

The results show treatment with SLNP-IC1-TR12 for 6 doses for 2 weeks produces anti-tumor activity when compared with the vehicle-treated group. The TGI was at 53.38%. (See, FIG. 31).

### Example 24: In Vivo Validation of SLNP-IC1-TR12 Efficacy in Neuro2A Neuroblastoma Tumor Model

In another experiment, in vivo efficacy/activity of SLNP-IC1-TR12 was confirmed using the following experiment(s). Briefly, murine neuroblastoma Nuero2A cells (1×10⁶) were inoculated subcutaneously in the right flank region of A/J mice. Animals were treated with vehicle control or SLNP-IC1-TR12 at 2.0/0.25 mg/kg, two (2) times a week through iv injection. Tumor volumes were measured three (3) times in two dimensions using a caliper, and the volume was calculated using the formula: V=(L×W×W)×0.5, where V is tumor volume, L is tumor length (the longest tumor dimension), and W is tumor width (the longest tumor dimension perpendicular to L). Body weight was measured three times a week. The tumor growth inhibition (TGI) was calculated based on the tumor size data of day 10.

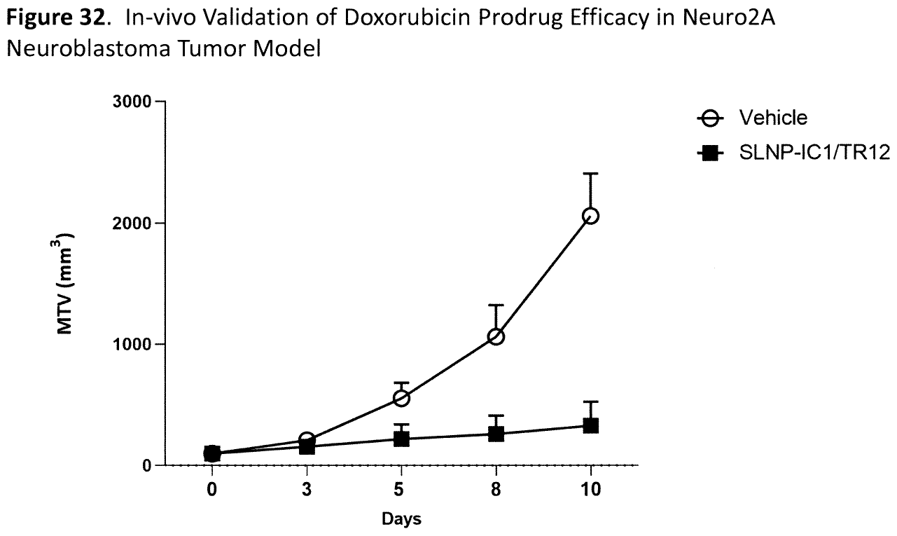

The results show, treatment with SLNP-IC1-TR12 for 6 doses for 2 weeks produces anti-tumor activity when compared with the vehicle-treated group. The TGI was at 88.27%. (See, FIG. 32).

### Example 25: In Vivo Validation of SLNP-IC1-TR12 Efficacy in CT26 Colon Tumor Model

In another experiment, in vivo efficacy/activity of SLNP-IC1-TR12 was confirmed using the following experiment(s). Briefly, murine colon CT26 cells (1×10⁶) were inoculated subcutaneously in the right flank region of Balb/c mice. Animals were treated with vehicle control or SLNP-IC1-TR12 at 2.0/0.25 mg/kg two (2) times a week through iv injection. Tumor volumes were measured three (3) times in two dimensions using a caliper, and the volume was calculated using the formula: V=(L×W×W)×0.5, where V is tumor volume, L is tumor length (the longest tumor dimension), and W is tumor width (the longest tumor dimension perpendicular to L). Body weight was measured three times a week. The tumor growth inhibition (TGI) was calculated based on the tumor size data of day 12.

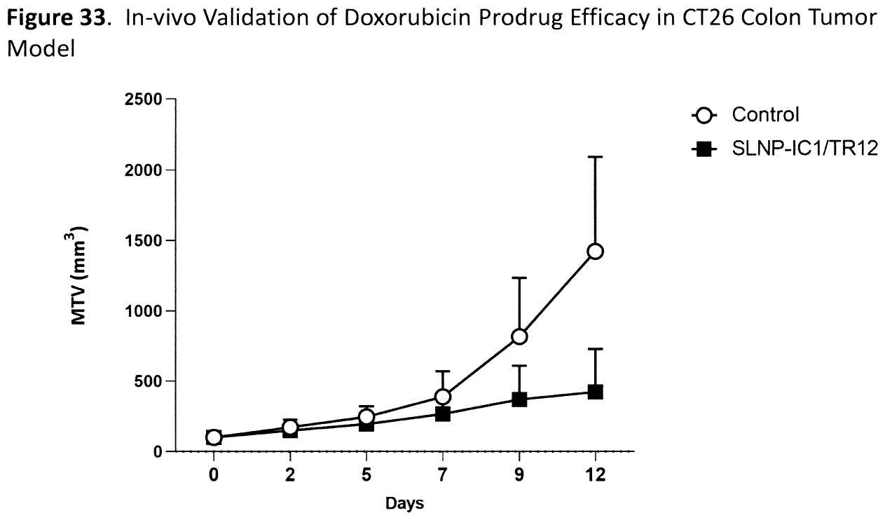

The results show, treatment with SLNP-IC1-TR12 for 6 doses for 2 weeks produce anti-tumor activity when compared with the vehicle-treated group. The TGI was at 81.16%. (See, FIG. 33).

### Example 26: In Vivo Validation of SLNP-IC1-TR12 Efficacy in MC38 Colon Tumor Model

In another experiment, in vivo efficacy/activity of SLNP-IC1-TR12 was confirmed using the following experiment(s). Briefly, murine colon MC38 cells (1×10⁶) were inoculated subcutaneously in the right flank region of C57/bl6 mice. Animals were treated with vehicle control or SLNP-IC1-TR12 at 2.0/0.25 mg/kg two (2) times a week through iv injection. Tumor volumes were measured three (3) times in two dimensions using a caliper, and the volume was calculated using the formula: V=(L×W×W)×0.5, where V is tumor volume, L is tumor length (the longest tumor dimension), and W is tumor width (the longest tumor dimension perpendicular to L). Body weight was measured three times a week. The tumor growth inhibition (TGI) was calculated based on the tumor size data of day 18.

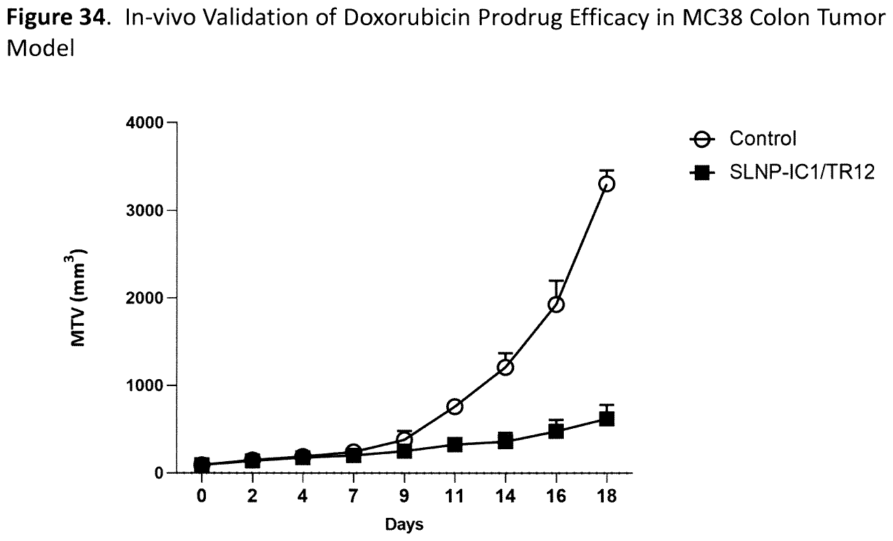

The results show, treatment with SLNP-IC1-TR12 for 6 doses for 2 weeks produce anti-tumor activity when compared with the vehicle-treated group. The TGI was at 83.59%. (See, FIG. 34).

### Example 27: In Vivo Validation of SLNP-IC1-TR12 Efficacy in Renca Kidney Tumor Model

In another experiment, in vivo efficacy/activity of SLNP-IC1-TR12 was confirmed using the following experiment(s). Briefly, murine Kidney Renca cells (1×10⁶) were inoculated subcutaneously in the right flank region of Balb/C mice. Animals were treated with vehicle control or SLNP-IC1-TR12 at 2.0/0.25 mg/kg two (2) times a week through iv injection. Tumor volumes were measured three (3) times in two dimensions using a caliper, and the volume was calculated using the formula: V=(L×W×W)×0.5, where V is tumor volume, L is tumor length (the longest tumor dimension), and W is tumor width (the longest tumor dimension perpendicular to L). Body weight was measured three times a week. The tumor growth inhibition (TGI) was calculated based on the tumor size data of day 13.

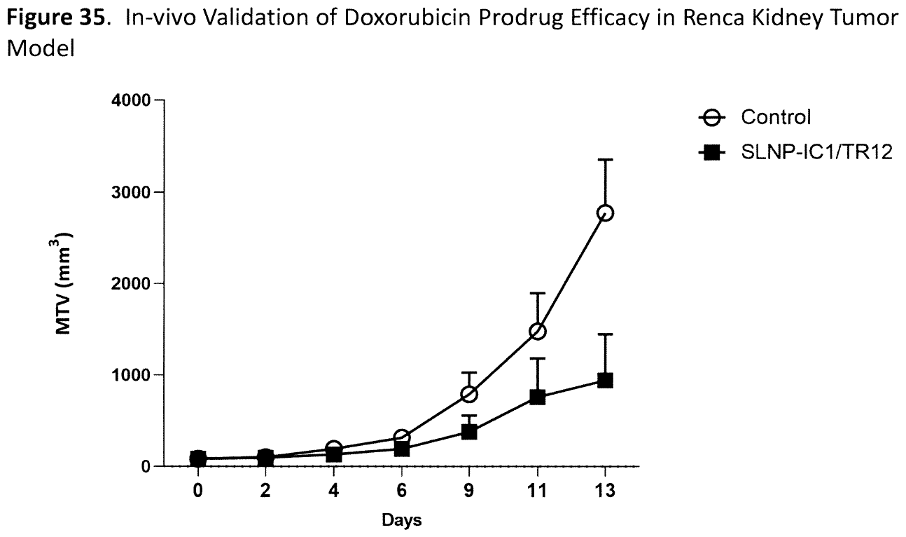

The results show, treatment with SLNP-IC1-TR12 for 6 doses for 2 weeks produce anti-tumor activity when compared with the vehicle-treated group. The TGI was at 68.18%. (See, FIG. 35).

### Example 28: In Vivo Validation of SLNP-IC1-TR12 Efficacy in H22 Liver Tumor Model

In another experiment, in vivo efficacy/activity of SLNP-IC1-TR12 was confirmed using the following experiment(s). Briefly, murine liver H22 cells (2×10⁶) were inoculated subcutaneously in the right flank region of Balb/C mice. Animals were treated with vehicle control or SLNP-IC1-TR12 at 2.0/0.25 mg/kg two (2) times a week through iv injection. Tumor volumes were measured three (3) times in two dimensions using a caliper, and the volume was calculated using the formula: V=(L×W×W)×0.5, where V is tumor volume, L is tumor length (the longest tumor dimension), and W is tumor width (the longest tumor dimension perpendicular to L). Body weight was measured three times a week. The tumor growth inhibition (TGI) was calculated based on the tumor size data of day 21.

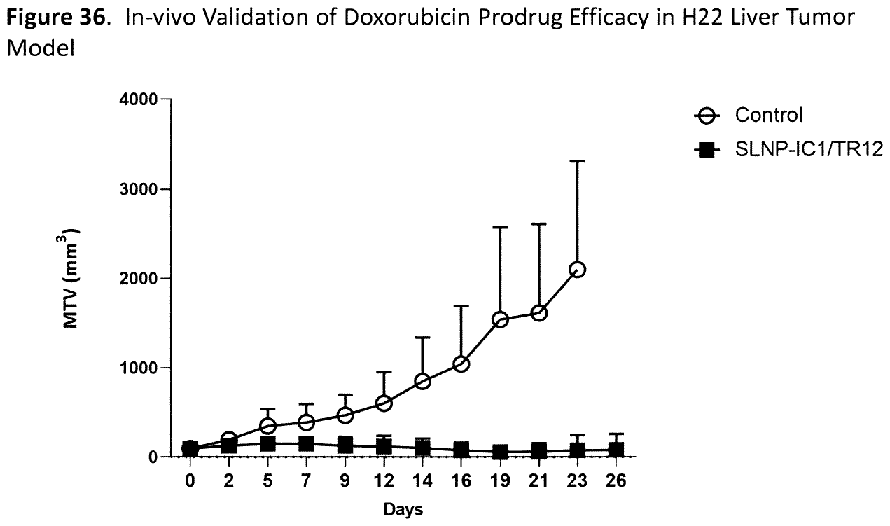

The results show, treatment with SLNP-IC1-TR12 for 6 doses for 2 weeks produce anti-tumor activity when compared with the vehicle-treated group. The TGI was at 100.96%. (See, FIG. 36).

### Example 29: In Vivo Validation of SLNP-IC1-TR12 Efficacy in Hepa1-6 Liver Tumor Model

In another experiment, in vivo efficacy/activity of SLNP-IC1-TR12 was confirmed using the following experiment(s). Briefly, murine liver Hepa 1-6 cells (5×10⁶) were inoculated subcutaneously in the right flank region of C57BL6 mice. Animals were treated with vehicle control or SLNP-IC1-TR12 at 2.0/0.25 mg/kg two (2) times a week through iv injection. Tumor volumes were measured three (3) times in two dimensions using a caliper, and the volume was calculated using the formula: V=(L×W×W)×0.5, where V is tumor volume, L is tumor length (the longest tumor dimension), and W is tumor width (the longest tumor dimension perpendicular to L). Body weight was measured three times a week. The tumor growth inhibition (TGI) was calculated based on the tumor size data of day 16.

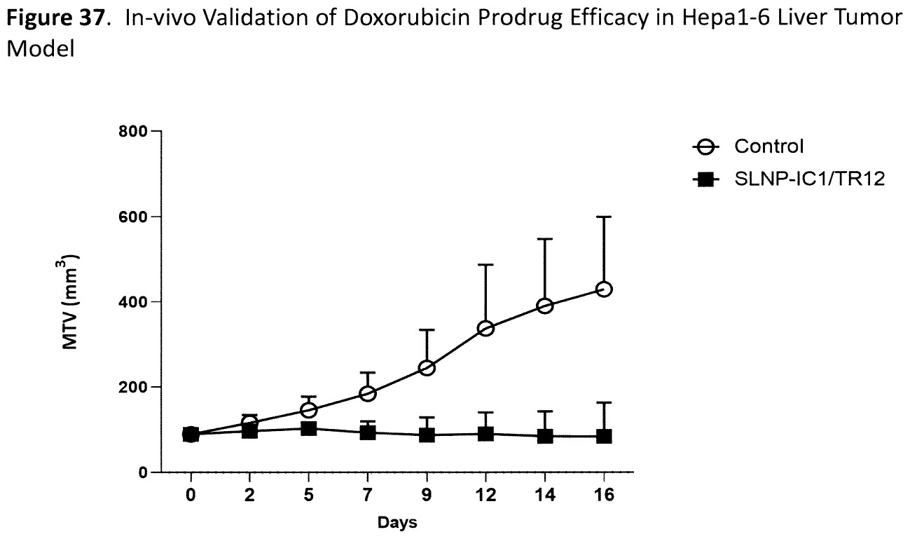

The results show, treatment with SLNP-IC1-TR12 for 6 doses for 2 weeks produce anti-tumor activity when compared with the vehicle-treated group. The TGI was at 100.47%. (See, FIG. 37).

### Example 30: In Vivo Validation of SLNP-IC1-TR12 Efficacy in LLC1 Lung Tumor Model

In another experiment, in vivo efficacy/activity of SLNP-IC1-TR12 was confirmed using the following experiment(s). Briefly, murine lung LLC1 cells (0.3 ×10⁶) were inoculated subcutaneously in the right flank region of C57BL6 mice. Animals were treated with vehicle control or SLNP-IC1-TR12 at 2.0/0.25 mg/kg two (2) times a week through iv injection. Tumor volumes were measured three (3) times in two dimensions using a caliper, and the volume was calculated using the formula: V=(L×W×W)×0.5, where V is tumor volume, L is tumor length (the longest tumor dimension), and W is tumor width (the longest tumor dimension perpendicular to L). Body weight was measured three times a week. The tumor growth inhibition (TGI) was calculated based on the tumor size data of day 16.

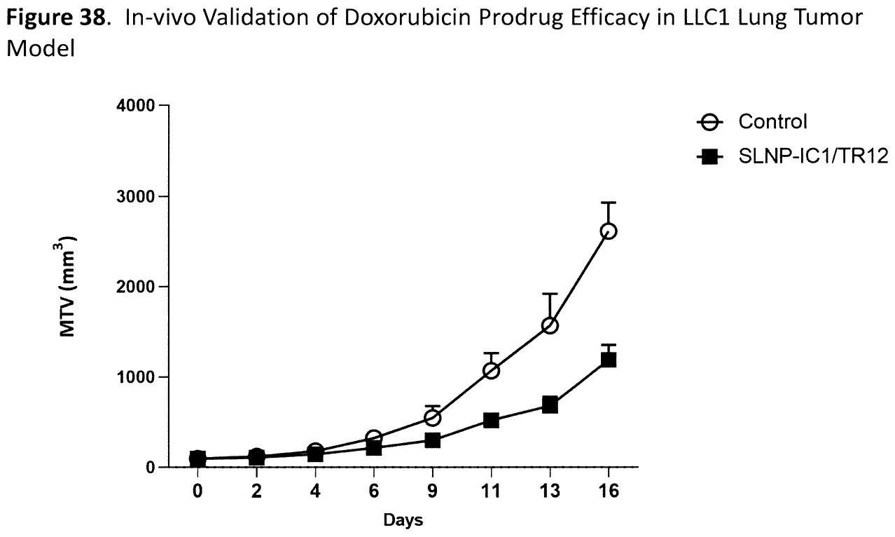

The results show, treatment with SLNP-IC1-TR12 for 6 doses for 2 weeks produce anti-tumor activity when compared with the vehicle-treated group. The TGI was at 56.65%. (See, FIG. 38).

### Example 31: In Vivo Validation of SLNP-IC1-TR12 Efficacy in KLN205 Lung Tumor Model

In another experiment, in vivo efficacy/activity of SLNP-IC1-TR12 was confirmed using the following experiment(s). Briefly, murine Lung KLN205 cells (0.8×10⁶) were inoculated subcutaneously in the right flank region of DBA/2 mice. Animals were treated with vehicle control or SLNP-IC1-TR12 at 2.0/0.25 mg/kg two (2) times a week through iv injection. Tumor volumes were measured three (3) times in two dimensions using a caliper, and the volume was calculated using the formula: V=(L×W×W)×0.5, where V is tumor volume, L is tumor length (the longest tumor dimension), and W is tumor width (the longest tumor dimension perpendicular to L). Body weight was measured three times a week. The tumor growth inhibition (TGI) was calculated based on the tumor size data of day 21.

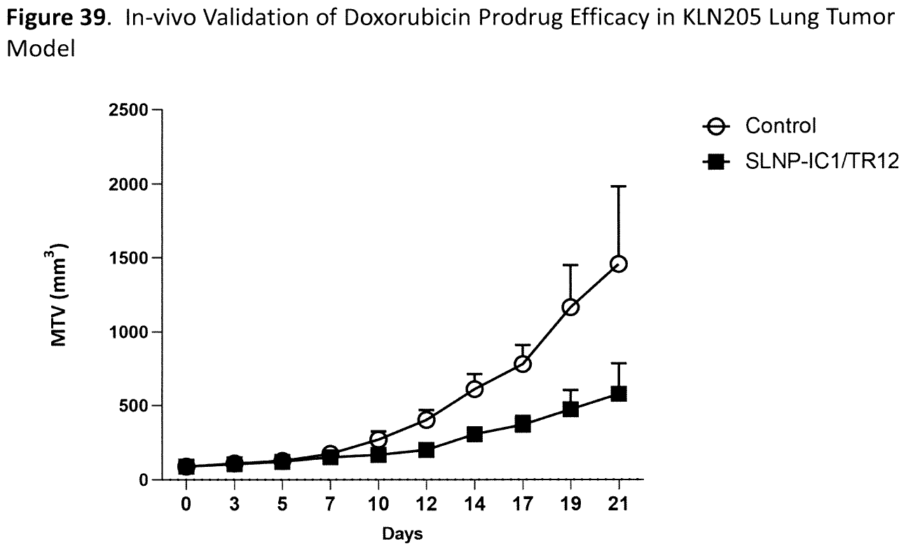

The results show, treatment with SLNP-IC1-TR12 for 6 doses for 2 weeks produce anti-tumor activity when compared with the vehicle-treated group. The TGI was at 64.21%. (See, FIG. 39).

### Example 32: In Vivo Validation of SLNP-IC1-TR12 Efficacy in B16BL6 Melanoma Tumor Model

In another experiment, in vivo efficacy/activity of SLNP-IC1-TR12 was confirmed using the following experiment(s). Briefly, murine melanoma B16Bl6 cells (0.2×10⁶) were inoculated subcutaneously in the right flank region of C57BL6 mice. Animals were treated with vehicle control or SLNP-IC1-TR12 at 2.0/0.25 mg/kg two (2) times a week through iv injection. Tumor volumes were measured three (3) times in two dimensions using a caliper, and the volume was calculated using the formula: V=(L×W×W)×0.5, where V is tumor volume, L is tumor length (the longest tumor dimension), and W is tumor width (the longest tumor dimension perpendicular to L). Body weight was measured three times a week. The tumor growth inhibition (TGI) was calculated based on the tumor size data of day 12.

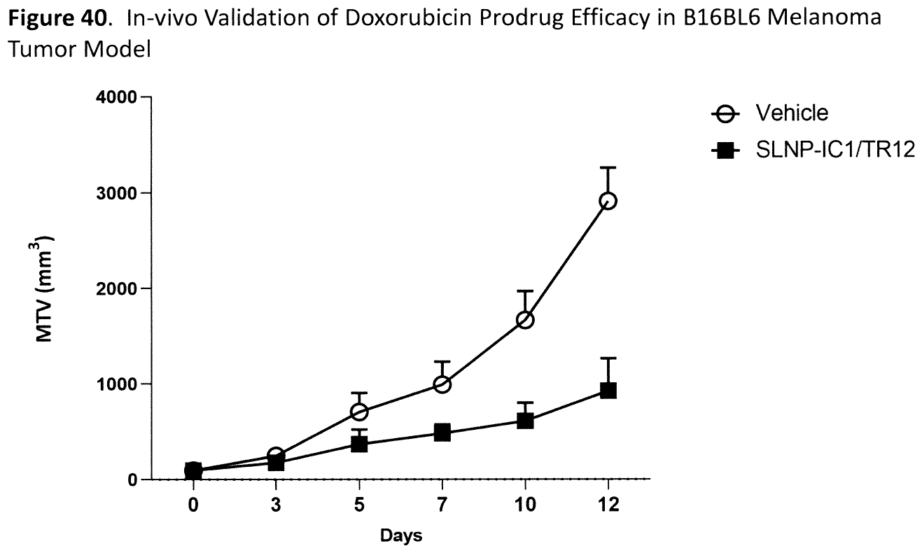

The results show, treatment with SLNP-IC1-TR12 for 6 doses for 2 weeks produce anti-tumor activity when compared with the vehicle-treated group. The TGI was at 70.51%. (See, FIG. 40).

### Example 33: In Vivo Validation of SLNP-IC1-TR12 Efficacy in Pan02.03 Pancreatic Tumor Model

In another experiment, in vivo efficacy/activity of SLNP-IC1-TR12 was confirmed using the following experiment(s). Briefly, murine pancreatic Pan02.03 cells (3×10⁶) were inoculated subcutaneously in the right flank region of C57BL6 mice. Animals were treated with vehicle control or SLNP-IC1-TR12 at 2.0/0.25 mg/kg two (2) times a week through iv injection. Tumor volumes were measured three (3) times in two dimensions using a caliper, and the volume was calculated using the formula: V=(L×W×W)×0.5, where V is tumor volume, L is tumor length (the longest tumor dimension), and W is tumor width (the longest tumor dimension perpendicular to L). Body weight was measured three times a week. The tumor growth inhibition (TGI) was calculated based on the tumor size data of day 62.

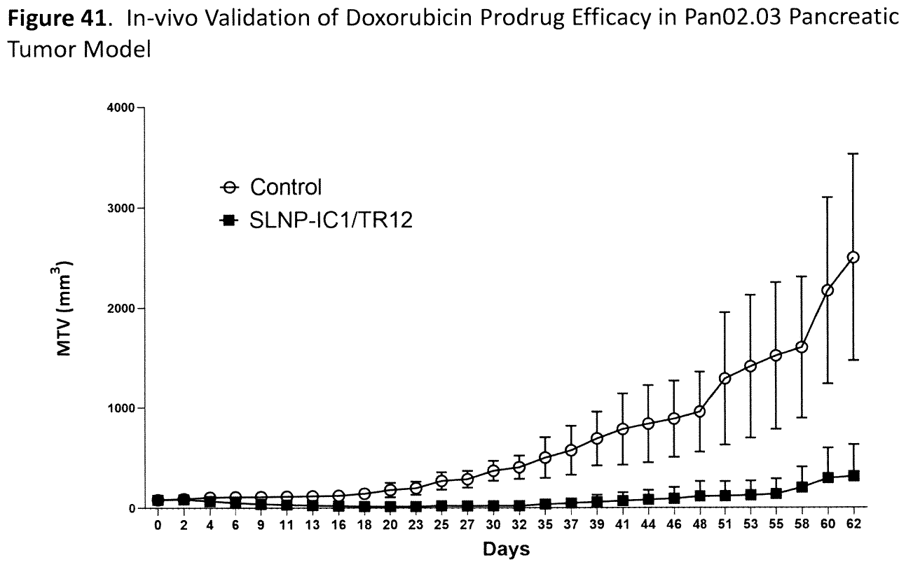

The results show, treatment with SLNP-IC1-TR12 for 6 doses for 2 weeks produce anti-tumor activity when compared with the vehicle-treated group. The TGI was at 90.48%. (See, FIG. 41).

### Example 34: In Vivo Validation of SLNP-IC1-TR12 Efficacy in RM-1 Prostate Tumor Model

In another experiment, in vivo efficacy/activity of SLNP-IC1-TR12 was confirmed using the following experiment(s). Briefly, murine prostate RM-1 cells (1×10⁶) were inoculated subcutaneously in the right flank region of C57BL6 mice. Animals were treated with vehicle control or SLNP-IC1-TR12 at 2.0/0.25 mg/kg two (2) times a week through iv injection. Tumor volumes were measured three (3) times in two dimensions using a caliper, and the volume was calculated using the formula: V=(L×W×W)×0.5, where V is tumor volume, L is tumor length (the longest tumor dimension), and W is tumor width (the longest tumor dimension perpendicular to L). Body weight was measured three times a week. The tumor growth inhibition (TGI) was calculated based on the tumor size data of 14.

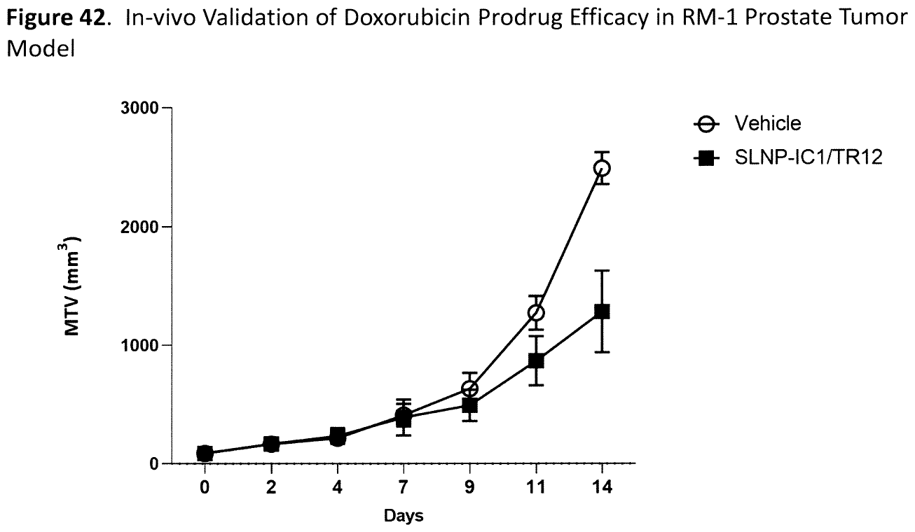

The results show, treatment with SLNP-IC1-TR12 for 6 doses for 2 weeks produce anti-tumor activity when compared with the vehicle-treated group. The TGI was at 50.24%. (See, FIG. 42).

### Example 35: In Vivo Validation of SLNP-IC1-TR12 Efficacy in BMT2 Bladder Tumor Model

In another experiment, in vivo efficacy/activity of SLNP-IC1-TR12 was confirmed using the following experiment(s). Briefly, murine bladder BMT2 cells (4×10⁶) were inoculated subcutaneously in the right flank region of C3H/He mice. Animals were treated with vehicle control or SLNP-IC1-TR12 at 2.0/0.25 mg/kg two (2) times a week through iv injection. Tumor volumes were measured three (3) times in two dimensions using a caliper, and the volume was calculated using the formula: V=(L×W×W)×0.5, where V is tumor volume, L is tumor length (the longest tumor dimension) and W is tumor width (the longest tumor dimension perpendicular to L). Body weight was measured three times a week. The tumor growth inhibition (TGI) was calculated based on the tumor size data of 16.

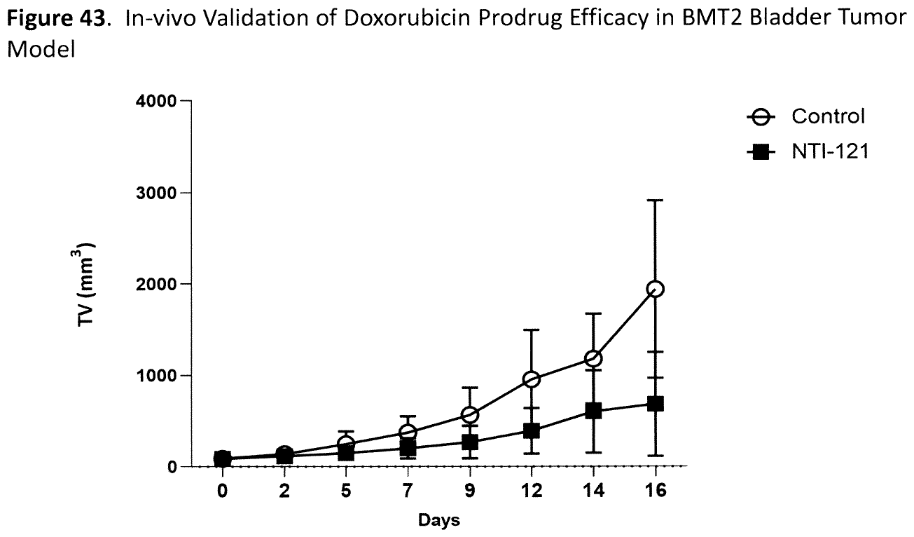

The results show, treatment with SLNP-IC1-TR12 for 6 doses for 2 weeks produce anti-tumor activity when compared with the vehicle-treated group. The TGI was at 67.81%. (See, FIG. 43).

### Example 36: In Vivo Validation of SLNP-IC1-TR12 Efficacy in Clone M-3 Melanoma Tumor Model

In another experiment, in vivo efficacy/activity of SLNP-IC1-TR12 was confirmed using the following experiment(s). Briefly, murine melanoma Clone M-3cells (0.2×10⁶) were inoculated subcutaneously in the right flank region of C57BL6 mice. Animals were treated with vehicle control or SLNP-IC1-TR12 at 2.0/0.25 mg/kg two (2) times a week through iv injection. Tumor volumes were measured three (3) times in two dimensions using a caliper, and the volume was calculated using the formula: V=(L×W×W)×0.5, where V is tumor volume, L is tumor length (the longest tumor dimension), and W is tumor width (the longest tumor dimension perpendicular to L). Body weight was measured three times a week. The tumor growth inhibition (TGI) was calculated based on the tumor size data of 12.

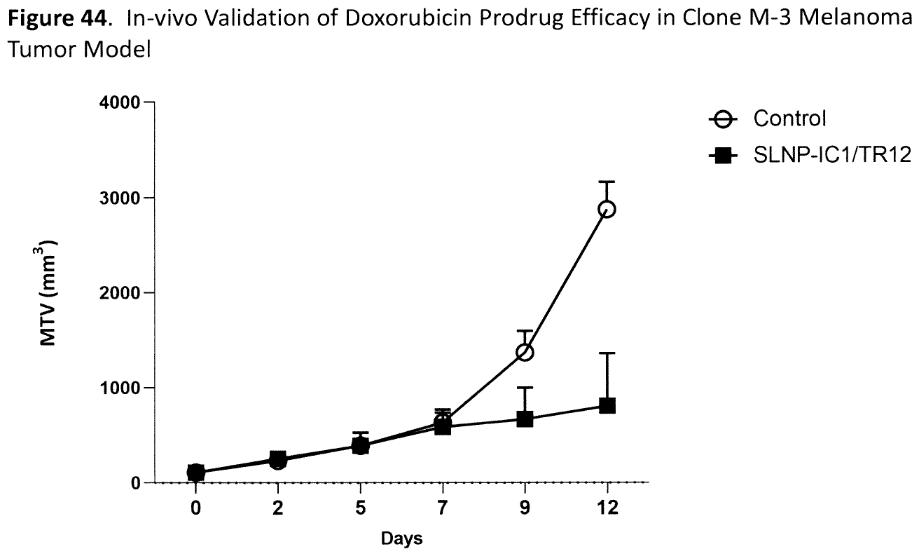

The results show, treatment with SLNP-IC1-TR12 for 6 doses for 2 weeks produce anti-tumor activity when compared with the vehicle-treated group. The TGI was at 74.88%. (See, FIG. 44).

### Example 37: In Vivo Validation of SLNP-IC1-TR12 Efficacy in 4T1 Breast Orthotopic Tumor Model

In another experiment, in vivo efficacy/activity of SLNP-IC1-TR12 was confirmed using the following experiment(s). Briefly, murine 4T1 breast cancer cells were inoculated mammary fat pads pf female Balb/c mice. Animals were treated with vehicle control or SLNP-IC1-TR12 at 2.0/0.25 mg/kg two (2) times a week through iv injection. Tumor volumes were measured three (3) times in two dimensions using a caliper, and the volume was calculated using the formula: V=(L×W×W)×0.5, where V is tumor volume, L is tumor length (the longest tumor dimension), and W is tumor width (the longest tumor dimension perpendicular to L). Body weight was measured three times a week. The tumor growth inhibition (TGI) was calculated based on the tumor size data of 26.

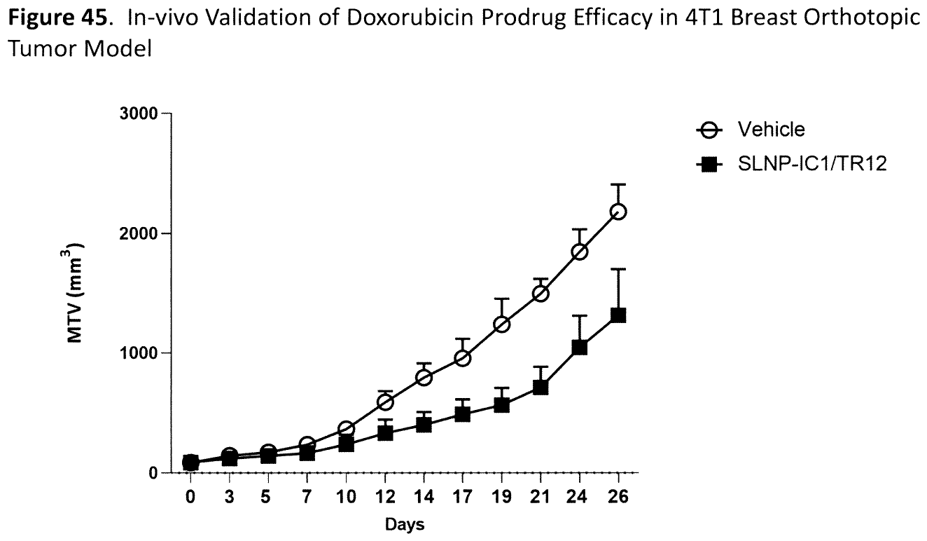

The results show, treatment with SLNP-IC1-TR12 for 6 doses for 2 weeks produce anti-tumor activity when compared with the vehicle-treated group. The TGI was at 41.34%. (See, FIG. 45).

### Example 38: In Vivo Validation of Several SLNP-TR12 Embodiments Alone or in Combination with SLNP-IC1 and/or SLNP-IC1-TR12 Efficacy in CT-26 Colon Tumor Model

In another experiment, in vivo efficacy/activity of SLNP-TR12 alone or in combination with SLNP-IC1 and/or SLNP-IC1-TR12 was confirmed using the following experiment(s). Briefly, murine colon cancer CT-26 cells (0.5×10⁶) were inoculated subcutaneously in the right rear flank region of Balb/c mice. Animals were treated with vehicle control and SLNP-TR12 at 0.25 mg/kg, SLNP-IC1 at 2.0 mg/kg, and SLNP-IC1-TR12 at 2.0/0.25 mg/kg, bi-weekly through iv injection. Tumor volumes were measured three (3) times in two dimensions using a caliper, and the volume was calculated using the formula: V=(L×W×W)×0.5, where V is tumor volume, L is tumor length (the longest tumor dimension), and W is tumor width (the longest tumor dimension perpendicular to L).

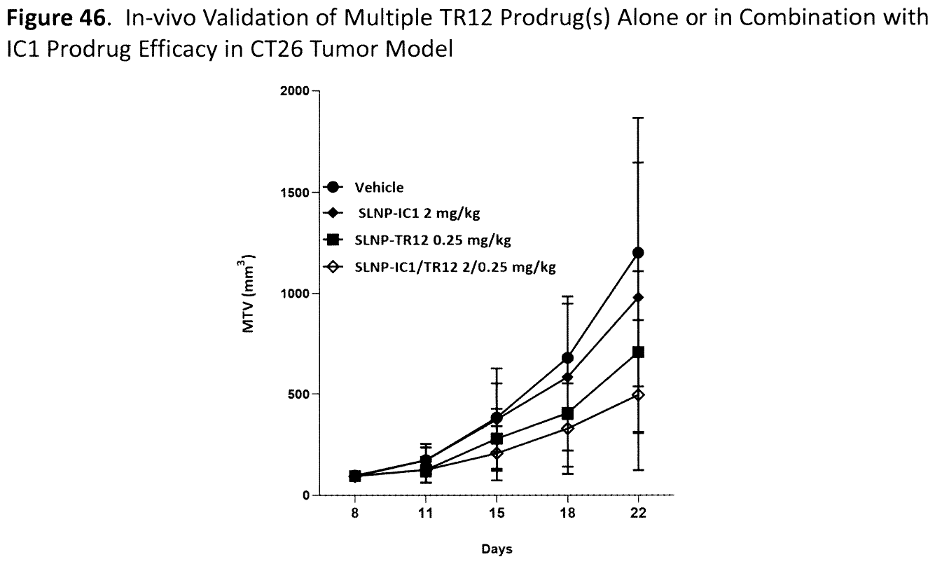

The results show that the combination treatment with SLNP-IC1-TR12 induces higher tumor growth inhibition when compared to single agent treatments. (See, FIG. 46).

### Example 39: In Vivo Validation of Several SLNP-TR12 Embodiments Alone or in Combination with SLNP-IC1 and/or SLNP-IC1-TR12 Efficacy in B16F10 Colon Tumor Model

In another experiment, in vivo efficacy/activity of SLNP-TR12 alone or in combination with SLNP-IC1 and/or SLNP-IC1-TR12 was confirmed using the following experiment(s). Briefly, murine colon cancer B16F10 cells (0.3×10⁶) were inoculated subcutaneously in the right rear flank region of Balb/c mice. Animals were treated with vehicle control and SLNP-TR12 at 0.25 mg/kg, SLNP-IC1 at 2.0 mg/kg, and SLNP-IC1-TR12 at 2.0/0.25 mg/kg, bi-weekly through iv injection. Tumor volumes were measured three (3) times in two dimensions using a caliper, and the volume was calculated using the formula: V=(L×W×W)×0.5, where V is tumor volume, L is tumor length (the longest tumor dimension), and W is tumor width (the longest tumor dimension perpendicular to L).

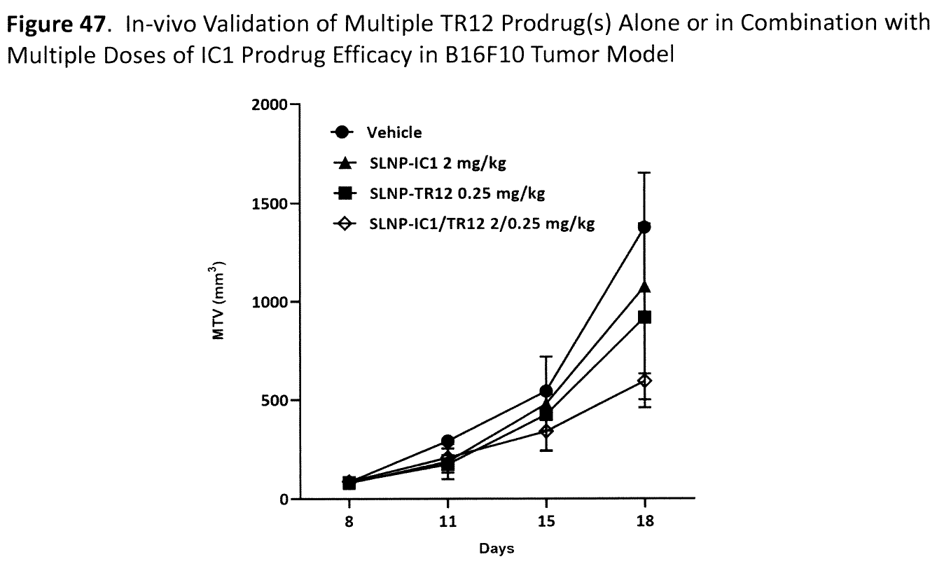

The results show that the combination treatment with SLNP-IC1-TR12 induces higher tumor growth inhibition when compared to single agent treatments. (See, FIG. 47).

### Example 40: In Vivo Validation of Several SLNP-TR12 Embodiments Alone or in Combination with SLNP-IC1 and/or SLNP-IC1-TR12 Efficacy in EMT-6 Tumor Model

In another experiment, in vivo efficacy/activity of SLNP-TR12 alone or in combination with SLNP-IC1 and/or SLNP-IC1-TR12 was confirmed using the following experiment(s). Briefly, murine breast cancer EMT-6 cells (0.5×10⁶) were inoculated subcutaneously in the right rear flank region of Balb/c mice. Animals were treated with vehicle control and SLNP-TR12 at 0.25 mg/kg, SLNP-IC1 at 2.0 mg/kg, and SLNP-IC1-TR12 at 2.0/0.25 mg/kg, bi-weekly through iv injection. Tumor volumes were measured three (3) times in two dimensions using a caliper, and the volume was calculated using the formula: V=(L×W×W)×0.5, where V is tumor volume, L is tumor length (the longest tumor dimension), and W is tumor width (the longest tumor dimension perpendicular to L).

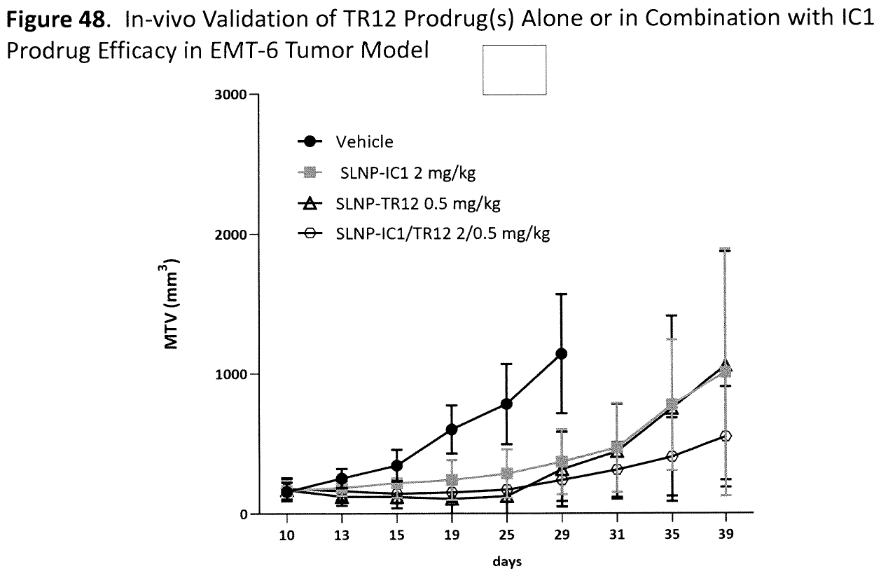

The results show that the combination treatment with SLNP-IC1-TR12 shows improved survival and TGI after the last dose of treatment when compared to single agent treatments. (See, FIG. 48).

### Example 41: Ex Vivo Validation of CD47 Ability to Block Cellular Uptake of SLNP-TR12

In another experiment, ex vivo validation of CD47 ability to block cellular uptake was confirmed using the following experiment(s). Briefly, and by way of background, it is understood that SLNP-TR12 induces cytokine secretion in PBMCs and Splenocytes. Notably, it is understood that certain cell populations in PBMCs and Splenocytes express ligands for CD47. Accordingly, the addition of CD47 to the SLNP-TR12 formulation should decrease the TR12 update and cytokine secretion.

To validate the blocking effects of a CD47 mimicry peptide on cellular update blocking, PBMCs, and Splenocytes were isolated from Balb/c mice. The cells were cultured at 2×10⁶/ml and treated with SLNP-TR12, SLNP-TR12-47c, and SLNP-TR12-47d for 24 hours. After 24 hours the culture was collected, and the TNF-a and IFN-g cytokine secretion was measured using ELISA following standard protocols.

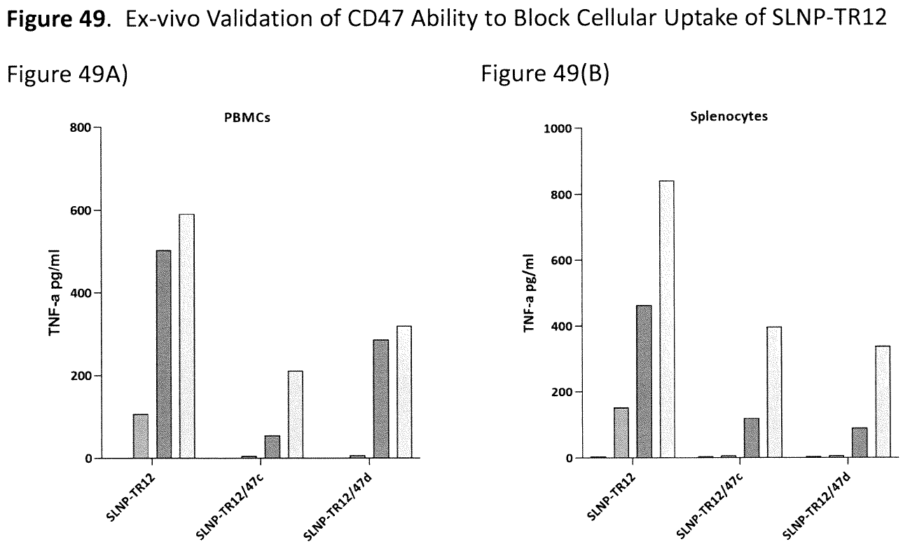

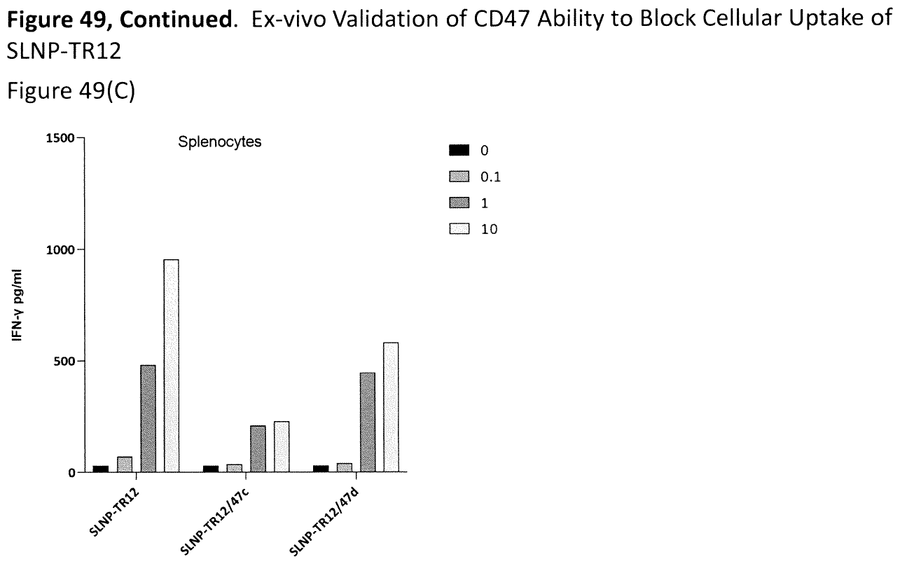

The results show lower secretion of TNFα and IFNγ in groups treated with SLNP-TR12-47c or SLNP-TR12/47d in both PBMCs (FIG. 49(A)) and Splenocytes (FIG. 49(B) & FIG. 49(C)) when compared to SLNP-TR12. Taken together, these results validate the uptake-blocking effect of a CD47 mimicry peptide. (See, FIG. 49).

### Example 42: Ex Vivo Validation of Immunomodulatory Effects of SLNP-TR12 on Tumor Infiltrating Lymphocytes in Balb/C Mice

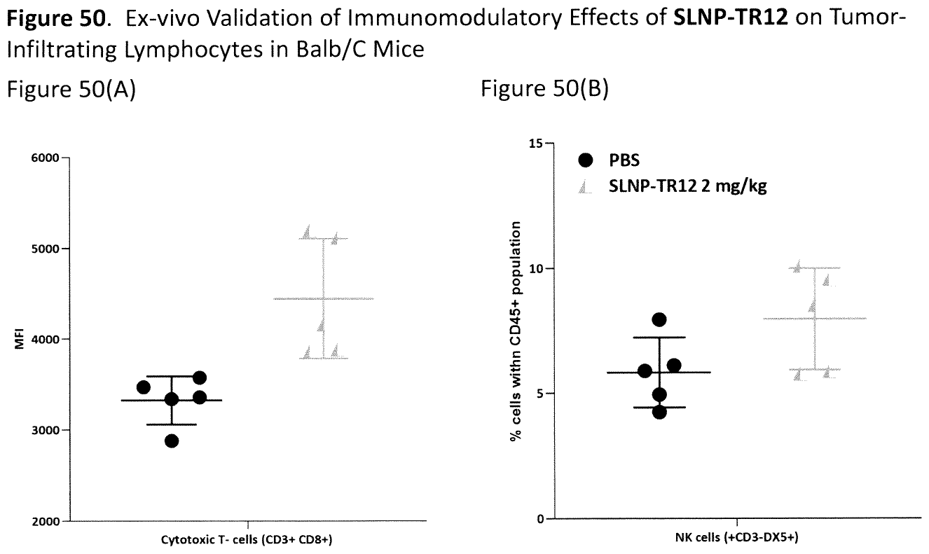

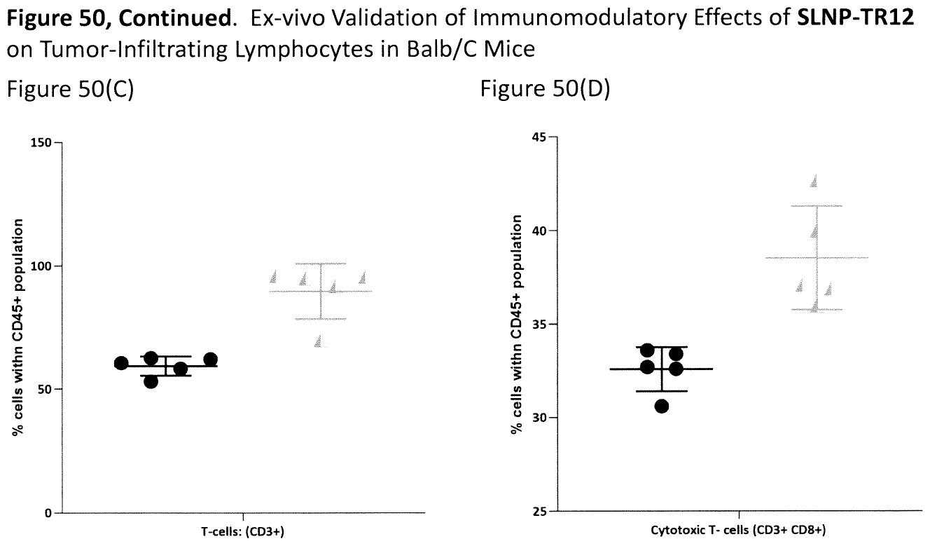

In another experiment, the immunomodulatory effects of SLNP-TR12 were confirmed using the following experiment(s). Briefly, Balb/C mice were treated with vehicle control or SLNP-TR12 at 2.0 mg/kg. Then, at four (4) hour post injection the spleens were harvested and the splenocytes were isolated. The splenocytes were stained and washed and the percentage (%) of different immune cells and Mean Fluorescence Channel (MFI) (FIG. 50(A)) were analyzed by flow cytometry using standard methods. The data presented shows the population gated from the viable and CD45+cells.

The results show that treatment with SLNP-TR12 increased the NK cells (FIG. 50(B)), total T-cells, (FIG. 50(C)), and cytotoxic T-cells (FIG. 50(0)). (See, FIG. 50).

### Example 43: In Vitro Validation of CD47 Ability to Block Cellular Uptake of SLNP-TR12

In another experiment, the ability of CD47 to block cellular uptake was confirmed using the following experiment(s). Briefly, Raw-Blue Reporter cell line was used. By way of background, it is understood that Raw Blue™ expresses human TLRs and an NF-κB/AP-1-inducible SEAP (secreted embryonic alkaline phosphatase) reporter gene. Thus, stimulation of these cells with TR12 can lead to NF-κB activation through TLR7 or TLR7/8 which can be measured by the detection of SEAP levels. Since these cell lines are macrophages, they express the ligand for CD45. Thus, by adding CD47 to the SLNP-TR12 formulation, this should decrease the uptake of the SLNP (SLNP-TR12) and decrease the SEAP secretion.

Accordingly, Raw-Blue cells were incubated with SLNP-TR12, SLNP-TR12-47c, or SLNP-TR12-47d at different concentrations. At 24-hour incubation, the TLR stimulation was assessed by measuring the levels of SEAP optical density (OD) using QUANTI-Blue™ via standard protocols. ODs were normalized to the control (untreated) group using appropriate methods.

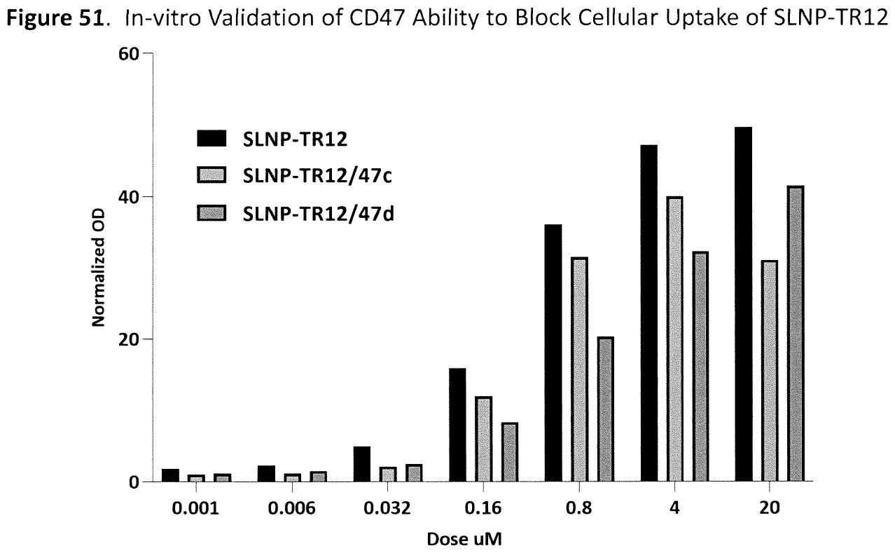

The results showed lower secretion of SEAP in the groups treated with SLNP-TR12-47c and SLNP-TR1247-d when compared to SLNP-TR12. (See, FIG. 51).

### Example 44: Human Clinical Trials for the Treatment of Human Carcinomas through the Use of Formulated and/or Co-Formulated Nanocarriers Comprising TLR Prodrugs (e.g., TR12)

Formulated and/or co-formulated nanocarriers containing TLR prodrugs and/or TLR lipid moieties are used in accordance with the present invention which specifically accumulate in a tumor cell and are used in the treatment of certain tumors and other immunological disorders and/or other diseases. In connection with each of these indications, two clinical approaches are successfully pursued.

I.) Adjunctive therapy: In adjunctive therapy, patients are treated with formulated and/or co-formulated nanocarriers containing TLR prodrugs (for example, SLNP-TR12 or SLNP-TR13) in combination with a chemotherapeutic or pharmaceutical or biopharmaceutical agent or a combination thereof. Primary cancer targets are treated under standard protocols by the addition of formulated and/or co-formulated nanocarriers containing TLR prodrugs. Protocol designs address effectiveness as assessed by the following examples, including but not limited to, reduction in tumor mass of primary or metastatic lesions, increased progression free survival, overall survival, improvement of patient's health, disease stabilization, as well as the ability to reduce usual doses of standard chemotherapy and other biologic agents. These dosage reductions allow additional and/or prolonged therapy by reducing dose-related toxicity of the chemotherapeutic or biologic agent.

II.) Monotherapy: In connection with the use of the formulated and/or co-formulated nanocarriers containing TLR prodrugs in monotherapy of tumors, the formulated and/or co-formulated nanocarriers containing TLR prodrugs (for example, SLNP-TR12 or SLNP-TR13) are administered to patients without a chemotherapeutic or pharmaceutical or biological agent. In one embodiment, monotherapy is conducted clinically in end-stage cancer patients with extensive metastatic disease. Protocol designs address effectiveness as assessed by the following examples, including but not limited to, reduction in tumor mass of primary or metastatic lesions, increased progression free survival, overall survival, improvement of patient's health, disease stabilization, as well as the ability to reduce usual doses of standard chemotherapy and other biologic agents.

Dosage

Dosage regimens may be adjusted to provide the optimum desired response. For example, a single formulated and/or co-formulated nanocarrier containing TLR prodrugs (for example, SLNP-TR12 or SLNP-TR13) may be administered by using several divided doses over time or the dose may be proportionally reduced or increased as indicated by the exigencies of the therapeutic situation. “Dosage Unit Form” as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention is dictated by and directly dependent on (a) the unique characteristics of the formulated and/or co-formulated nanocarriers containing TLR prodrugs (for example, SLNP-TR12 or SLNP-TR13), (b) the individual mechanics of the combination compound, if any, (c) the particular therapeutic or prophylactic effect to be achieved, and (d) the limitations inherent in the art of compounding such a compound for the treatment of sensitivity in individuals.

Clinical Development Plan (CDP)

The CDP follows and develops treatments of using formulated and/or co-formulated nanocarriers containing TLR prodrugs (for example, SLNP-TR12 or SLNP-TR13) in connection with adjunctive therapy or monotherapy. Trials initially demonstrate safety and thereafter confirm efficacy in repeat doses. Trials are open label comparing standard chemotherapy and/or the current standard of therapy plus formulated and/or co-formulated nanocarriers containing TLR prodrugs. As will be appreciated, one non-limiting criteria that can be utilized in connection with enrollment of patients is expression of TLR in a tumor as determined by standard detection methods known in the art.

It is believed that formulated and/or co-formulated nanocarriers, or any of the embodiments disclosed herein, may possess a satisfactory pharmacological profile and promising biopharmaceutical properties, such as favorable toxicological profile, favorable metabolism and pharmacokinetic properties, solubility, and permeability. It will be understood that determination of appropriate biopharmaceutical properties is within the knowledge of a person skilled in the art (e.g., determination of cytotoxicity in cells or inhibition of certain targets or channels to determine potential toxicity).

The present invention is not to be limited in scope by the embodiments disclosed herein, which are intended as single illustrations of individual aspects of the invention, and any that are functionally equivalent are within the scope of the invention. Various modifications to the models, methods, and life cycle methodology of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

TABLE I
Examples of Lipids.

| No. | Abbreviation | Name/Chemical Formula |
|---|---|---|
| 1 | CHOL | Cholesterol |
| 2 | DPPG•Na | 1,2-dipalmitoyl-sn-glycero-3-phospho-(1′-rac-glycerol) (sodium salt) |
| 3 | DMPG•Na | 1,2-dimyristoyl-sn-glycero-3-phospho-(1′-rac-glycerol) (sodium salt) |
| 4 | Lyso PC | 1-decanoyl-2-hydroxy-sn-glycero-3-phosphocholine |
| 5 | (Δ9-Cis) PG | 1,2-dioleoyl-sn-glycero-3-phospho-(1′-rac-glycerol) (sodium salt) |
| 6 | Soy Lyso PC | L-α-lysophosphatidylcholine (Soy) |
| 7 | PG | 1,2-dilauroyl-sn-glycero-3-phospho-(1′-rac-glycerol) (sodium salt) |
| 8 | PA-PEG3-mannose | 1,2-dipalmitoyl-sn-glycero-3-phospho((ethyl-1′,2′,3′- |
| | | triazole)triethyleneglycolmannose) (ammonium salt) |
| 9 | C16 PEG2000 | N-palmitoyl-sphingosine-1-{succinyl[methoxy(polyethylene |
| | Ceramide | glycol)2000]} |
| 10 | MPLA | Monophosphoryl Lipid A |

TABLE II
Examples of Helper Lipids.

| No. | Abbreviation | Name |
|---|---|---|
| 1 | DOTAP | 1,2-dioleoyl-3-trimethylammonium-propane |
| | | (chloride salt) |
| 2 | DODMA | 1,2-dioleyloxy-3-dimethylaminopropane |
| 3 | DLinDMA | 1,2-dilinoleyloxy-3-dimethylaminopropane |
| 4 | DLin-KC2- | 2,2-dilinoleyl-4-dimethylaminoethyl-[1,3]- |
| | DMA | dioxolane |
| 5 | Δ9-Cis PE | 1,2-Dioleoyl-sn-glycero-3-phosphoethanolamine |
| | (DOPE) | |
| 6 | DOPC | 1,2-dioleoyl-sn-glycero-3-phosphocholine |
| 7 | CHOL | Cholesterol |
| 8 | PEG-C- | N-[(methoxy poly(ethylene glycol)2000)carbamyl]- |
| | DMA | 1,2-dimyristyloxlpropyl-3-amine |
| 9 | CHEMS | cholesteryl hemisuccinate |
| 10 | DPPC | 1,2-dipalmitoyl-sn-glycero-3-phosphocholine |
| 11 | DSPC | 1,2-distearoyl-sn-glycero-3-phosphocholine |
| 12 | MO-CHOL | 4-(2-aminoethyl)-morpholino- |
| | | cholesterolhemisuccinate |

TABLE III
Examples of Phospholipids/Fatty Acids.

| No. | Name |
|---|---|
| 1 | Oleic acid |
| 2 | linolenic acid |
| 3 | arachidonic acid |
| 4 | docosahexaenoic (DHA) |
| 5 | Palmitic acid |
| 6 | Palmitoleic acid |
| 7 | Stearic acid |
| 8 | Eicosapentaenoic acid (EPA) |
| 9 | DSPE-PEG(2000) Carboxylic Acid (1,2-distearoyl-sn-glycero-3- |
| | phosphoethanolamine-N-[carboxy(polyethylene glycol) |
| 10 | DOPE-PEG(2000) Carboxylic acid (1,2-dioleoyl-sn-glycero-3- |
| | phosphoethanolamine-N-[carboxy(polyethylene glycol)-2000] |
| | (sodium salt) |
| 11 | DMG-PEG 2000 1,2-dimyristoyl-rac-glycero-3- |
| | methoxypolyethylene glycol-2000 |

1. A TLR prodrug composition comprising,
(iv) a drug moiety;
(v) a lipid moiety; and
(vi) a linkage unit (“LU”),
whereby the drug moiety comprises a TLR agonist and whereby the LU conjugates the drug moiety with the lipid moiety, and wherein the drug moiety has the following chemical structure:

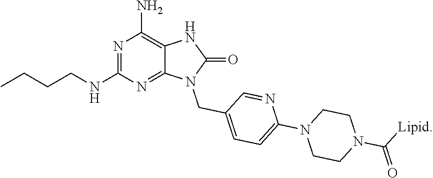

2. The TLR prodrug of claim 1, wherein the lipid moiety comprises a lipid set forth in Table I.

3. The TLR prodrug of claim 1, wherein the lipid moiety comprises a lipid set forth in Table III.

4. The TLR prodrug of claim 1, wherein the lipid moiety comprises Stearic Acid.

5. The TLR prodrug of claim 1, wherein the lipid moiety comprises Stearic Acid and has the following chemical structure:

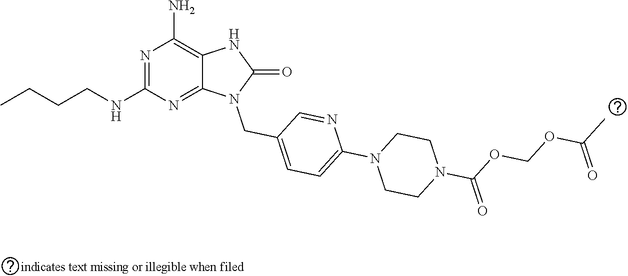 [SMILES: CCCCNc1nc(N)c2[nH]c(=O)n(Cc3ccc(N4CCN(C(=O)OCOC(C)=O)CC4)nc3)c2n1]

6. A nanocarrier comprising, an TLR prodrug whereby the nanocarrier releases an active TLR agonist after cleavage of a LU.

7. The nanocarrier of claim 6, wherein the LU is a hydromethylcarbamate linker.

8. The nanocarrier of claim 6, further comprising a helper lipid, whereby the helper lipid is set forth in Table II.

9. The nanocarrier of claim 6, wherein the TLR prodrug comprises TR12 and has the following chemical structure:

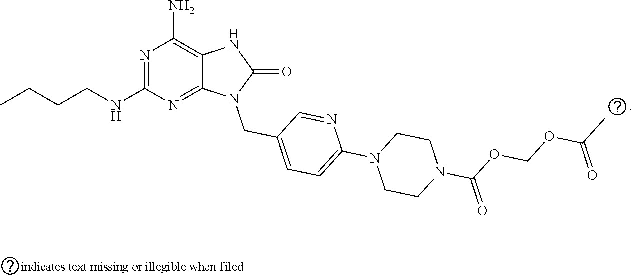 [SMILES: CCCCNc1nc(N)c2[nH]c(=O)n(Cc3ccc(N4CCN(C(=O)OCOC(C)=O)CC4)nc3)c2n1]

10. The nanocarrier of claim 9, wherein the nanocarrier is a solid-lipid nanoparticle (SLNP).

11. The SLNP of claim 10, wherein the TLR prodrug comprises TR12 and is denoted SLNP-TR12.

12. The SLNP of claim 11, whereby the SLNP is further co-formulated with one or more immune modulating agent or a lipid-prodrug thereof, wherein the immune modulating agent is selected from the group consisting of immunogenic-cell death inducing chemotherapeutics, A2aR inhibitors, STING agonists, CTLA-4 inhibitors, IDO inhibitors, PD-1/PD-L1 inhibitors, CD1D agonists and/or prodrugs thereof.

13. The SLNP of claim 11, whereby the SLNP is further co-formulated with an ICD-inducing chemotherapeutic, wherein the ICD-inducing chemotherapeutic is selected from the group consisting of DOX, MTO, OXA, CP, Bortezomib, Carfilzimib, IC1, or Paclitaxel.

14. The SLNP of claim 13, further comprising DOX.

15. A composition comprising a solid-lipid nanoparticle (SLNP) wherein the SLNP further comprises TR12 co-formulated with IC1 (denoted SLNP-TR12-IC1).

16. The composition of claim 15 co-formulated with IC1, wherein the ratio is 8:1 (denoted NTI-121).

17. The composition of claim 15 co-formulated with IC1, wherein the ratio is 16:1.

18. A kit comprising the composition of claim 15.


SEQUENCE LISTING (2 sequences)

SEQ ID NO: 1 - protein, 15 aa, Homo sapiens
GSGCERVIGTGWVRC

SEQ ID NO: 2 - protein, 12 aa, Homo sapiens
CERVIGTGWVRC